(19) United States
(12) Patent Application Publication
(10) Pub. No.: US 2024/0156950 A1
(43) Pub. Date: May 16, 2024

(54) EXTRACELLULAR VESICLE-BASED NANOCARRIERS

(71) Applicant: OHIO STATE INNOVATION FOUNDATION, Columbus, OH (US)

(72) Inventors: Natalia Higuita-Castro, Columbus, OH (US); Daniel Gallego-Perez, Columbus, OH (US); Silvia Durate-Sanmiguel, Columbus, OH (US); Ana Salazar-Puerta, Columbus, OH (US)

(21) Appl. No.: 18/547,976

(22) PCT Filed: Mar. 14, 2022
(86) PCT No.: PCT/US2022/071126

§ 371 Date: Aug. 25, 2023

Related U.S. Application Data
(60) Provisional application No. 63/161,093, filed on Mar. 15, 2021.

(51) Int. Cl. A61K 39/215; C07K 14/705; A61K 39/00

(52) U.S. Cl. CPC A61K 39/215; C07K 14/70525; A61K 2039/53; A61K 2039/55555; C07K 2319/03

(57) ABSTRACT

Disclosed herein is a system that engages skin-resident APCs by directly delivering a vaccine composition, and a system that turns skin cells into a vaccine dispatch center to amplify immunity via the production of engineered extracellular vesicles (EVs) functionalized with targeting ligands and loaded with the vaccine composition that can be targeted to extracutaneous APCs. In particular, disclosed herein is a vaccine composition that involves a first polynucleotide encoding or comprising a viral, bacterial, or tumor antigen, and a second polynucleotide encoding a fusion protein comprising an APC-targeting ligand and an exosomal or lysosomal transmembrane protein. Also disclosed is a method of vaccinating a subject that involves transfecting skin cells of the subject with the disclosed vaccine composition. Also disclosed herein is a method of vaccinating a subject that involves administering to the subject the disclosed EV vaccine.

### CROSS-REFERENCE TO RELATED APPLICATIONS

This application claims benefit of U.S. Provisional Application No. 63/161,093, filed Mar. 15, 2021, which is hereby incorporated herein by reference in its entirety.

### SEQUENCE LISTING

This application contains a sequence listing filed in electronic form as an ASCII.txt file entitled “321501_2540_Sequence_Listing_ST25” created on Feb. 16, 2022, having 366,068 bytes. The content of the sequence listing is incorporated herein in its entirety.

### BACKGROUND

DNA/mRNA vaccines have emerged as a promising alternative to traditional vaccines due to their ability to target acquired and innate immunity. While DNA/mRNA vaccines are actively being developed for COVID-19 (e.g., mRNA-1273), currently there is a scarcity of methods to deliver such vaccines into APCs in a targeted manner.

### SUMMARY

Disclosed herein is a system that engages skin-resident APCs by directly delivering a vaccine composition, and a system that turns skin cells into a vaccine dispatch center to amplify immunity via the production of engineered extracellular vesicles (EVs) functionalized with targeting ligands and loaded with the vaccine composition that can be targeted to extracutaneous APCs. In particular, disclosed herein is a vaccine composition that involves a first polynucleotide encoding or comprising a viral, bacterial, or tumor antigen, and a second polynucleotide encoding a fusion protein comprising an APC-targeting ligand and an exosomal or lysosomal transmembrane protein.

In some embodiments, the viral antigen is from a retrovirus, reovirus, rhabdovirus, poliovirus, potyvirus, geminivirus, flexivirus, picornavirus, togavirus, orthomyxovirus, paramyxovirus, calicivirus, arenavirus, flavivirus, filovirus, bunyavirus, coronavirus, astrovirus, adenovirus, papillomavirus, parvovirus, herpesvirus, hepadnavirus, poxvirus, or polyomavirus.

In some embodiments, the viral antigen is a SARS-CoV-2 antigen. For example, in some embodiments, the viral antigen is mRNA-1273 (Moderna, Inc.), AZD-1222 (AstraZeneca and University of Oxford), BNT162 (Pfizer and BioNTech), CoronaVac (Sinovac), NVX-CoV 2372 (NovoVax), SCB-2019 (Sanofi and GSK), ZyCoV-D (Zydus Cadila), or CoVaxin (Bharat Biotech), LV-SMENP-DC (Shenzhen Geno-Immune Medical Institute), CVnCoV (CureVac biopharmaceuticals), Gam-COVID-Vac Lyo/Sputnik V (Gamaleya Research Institute of Epidemiology and Microbiology), Ad5-nCoV (Cansino Biologics), DeINS1-SARS-CoV-2-RBD (University of Hong Kong), Coroflu (University ofWisconsin-Madison/Bharath Biotech Inc), PittCoVacc (University of Pittsburgh School of Medicine), Covid-19 aAPC (Shenzhen Geno-Immune Medical Institute), Triple Antigen Vaccine (Premas Biotech), or BBIBP-CorV (Beijing Institute of Biological Products and Sinopharm).

In some embodiments, the APC-targeting ligand comprises ICAM1 or ICAM4. In some embodiments, the APC-targeting ligand is selected from the group consisting of CD2, CD11a, CD18, CD22, CD29, CD40L, LDL, oxLDL, Lectins, Galectin 1, Galectin 3, Flagellin, Cxcl5, KRT14, FGF7, FGF10, and AMP-IBP5.

Design strategies for producing exosomes is described in Liu C, et al. Theranostics. 2019 9(4):1015-1028, which is incorporated by reference for the teaching of transmembrane proteins that can be used to guide fusion proteins into exosomes. Therefore, in some embodiments, the transmembrane protein is selected from the group consisting of CD63, CD9, CD81, CD53, CD82, CD37 (Tetraspanins), Alix (endosome-associated proteins), flotillin-1 (lipid raft-associated protein), TSG101 (Component of the ESCRT-I complex), ARRDC (Arrestin family of protein), Palmitoylated tdTomato (Tandem dimer Tomato fused at NH2-termini with a palmitoylation signal for EV membrane labelling), Lactadherin C1C2 domain (Membrane glycoprotein), EGF VIII (Transmembrane glycoprotein), PDGFR™ domain (Cell surface tyrosine kinase receptor), HIV-1 Nef (mut) (Released in extracellular vesicles), VSVG (Vesicular stomatitis virus glycoprotein), LAMP2B (Lysosome-Associated Membrane Glycoprotein 2), LAMP1 (Lysosome-Associated Membrane Glycoprotein 1), ALIX-1 (Cytosolic protein that associates with MVB by interacting with ESCRT-Ill subunit SNF7), HSP70 (Heat Shock Protein), HSP90 (Heat Shock Protein), MHC (Anchored in the membrane), SCAMPs (Secretory Carrier-Associated Membrane Protein 18), ApoE (Apolipoprotein E), and WW tag (Recognized by the L-domain-containing protein Ndfip1, resulting in ubiquitination and loading into exosomes).

In some embodiments, the first polynucleotide and the second polynucleotide are present in a single plasmid. In other embodiments, they are in separate polynucleotides that can be delivered simultaneously or sequentially.

Also disclosed is a method of vaccinating a subject that involves transfecting skin cells of the subject with the disclosed vaccine composition. As disclosed herein, this method will cause skin-resident skin cells to produce EVs containing the viral, bacterial, or tumor antigen and decorated on the surface with an APC-targeting ligand.

Also disclosed is an EV vaccine composition that involves EVs containing a viral, bacterial, or tumor antigen and/or containing a plasmid or oligonucleotide encoding the viral, bacterial, or tumor antigen, wherein the EVs are decorated on the surface with an APC-targeting ligand. Also disclosed herein is a method of vaccinating a subject that involves administering to the subject the disclosed EV vaccine.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows TNT- and exosome-driven vaccination methods against COVID-19. Panel (a) shows TNT is applied on the skin surface, near lymph nodes, to directly delivery DNA encoding for COVID19-specific antigens into skin-resident APCs. Panel (b) shows TNT-treated skin can also make engineered exosomes, decorated with ligands that can target APCs (i.e., ICAM1/4), and loaded with plasmid DNA and mRNA encoding for COVID19-specific antigens. These exosomes will be dispatched from the skin to target node-resident APCs, systemically, to amplify the immune response against COVID19-specific antigens. The TNT procedure is only applied once, and it lasts approximately 100 ms per application.

FIGS. 2A to 2J show ICAM1-decorated exosomes preferentially targeting CD11b+ myeloid cells and driving anti-tumor immunity. FIGS. 2A and 2B show notransfection of mouse embryonic fibroblasts (MEFs) which results in the release of exosomes with defined decoration and cargo. FIGS. 2D and 2E show how ICAM1-decorated exosomes drives preferential uptake by CD11 b+ myeloid cells, and FIG. 2F shows pro-inflammatory response depending on cargo. FIGS. 2G to 2J show engineered exosomes deployed by tail injection home to the tumor (FIG. 2G), and hinder progression (FIG. 2H) by (i-j) driving anti-tumoral immunity (FIGS. 21 to 2J).

FIGS. 3A to 3J show TNT drives in situ production of engineered exosomes with anti-tumoral activity from the skin. FIGS. 3A and 3B show TNT applied on skin that drive in situ release of ICAM1-decorated engineered exosomes. FIGS. 3C and 3D show anti-tumoral activity. FIG. 3E to 3J show targeting of myeloid cells within the tumor by ICAM1-decorated exosomes (FIG. 3E), and that the miR-146a and Glut1 cargo can induce an immune response hindering tumor burden (FIG. 3F to 3J).

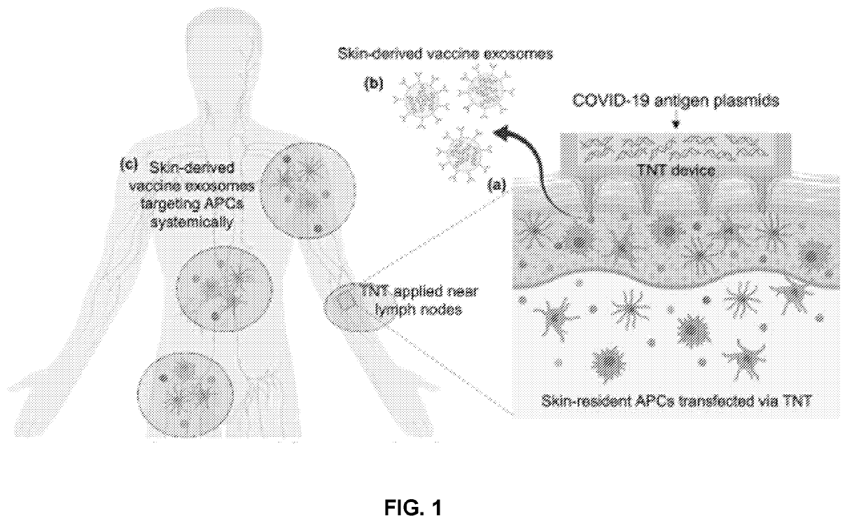

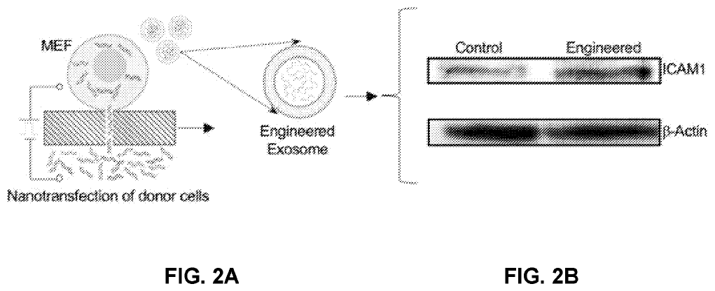

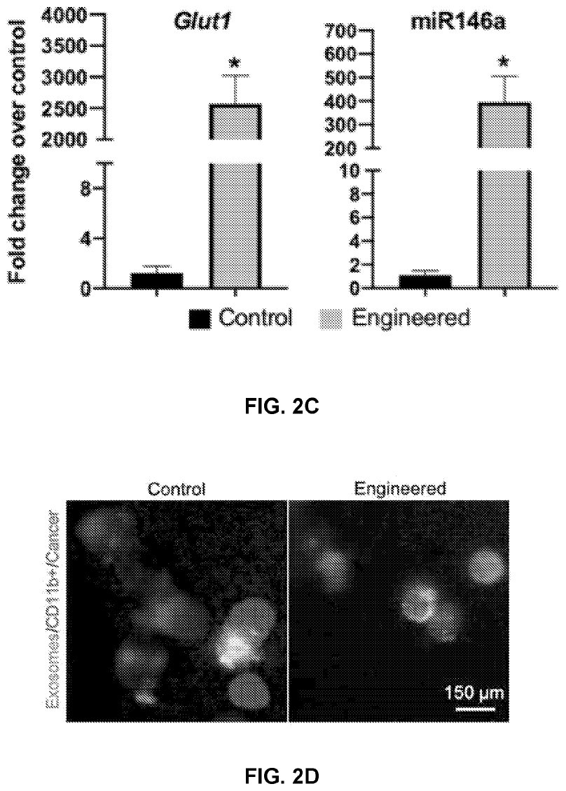

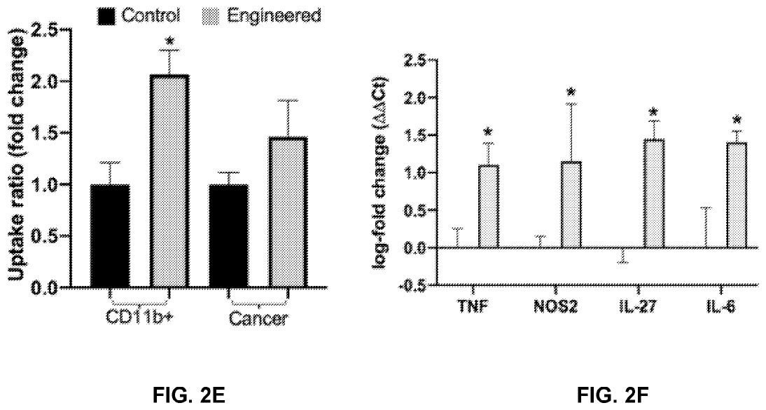

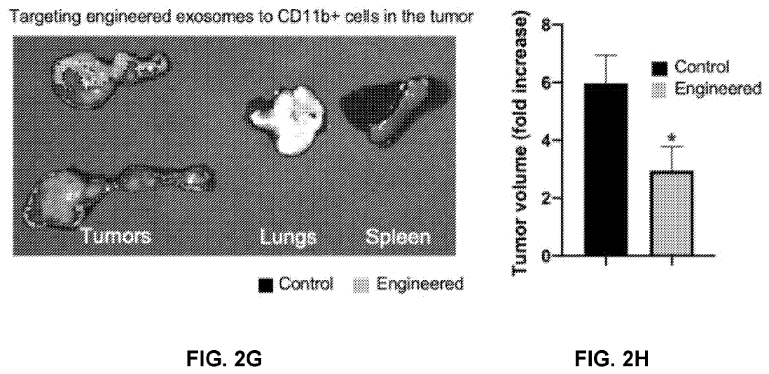

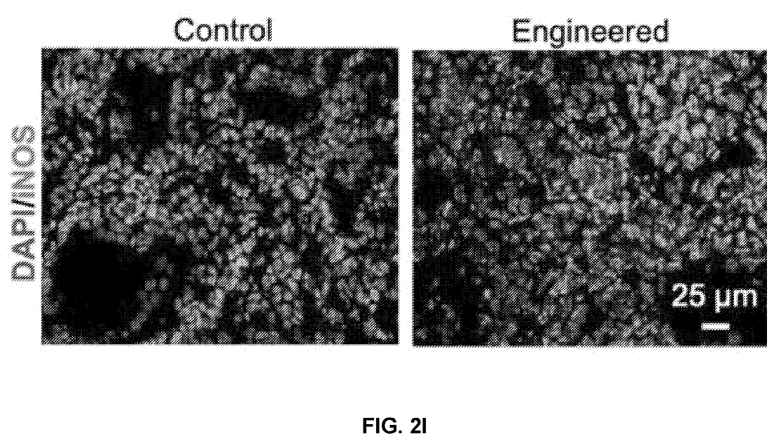

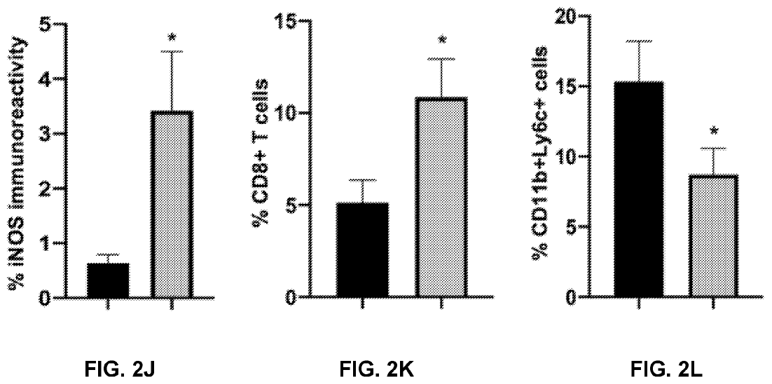

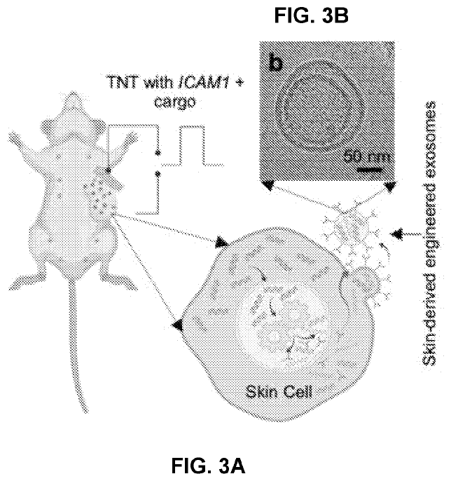

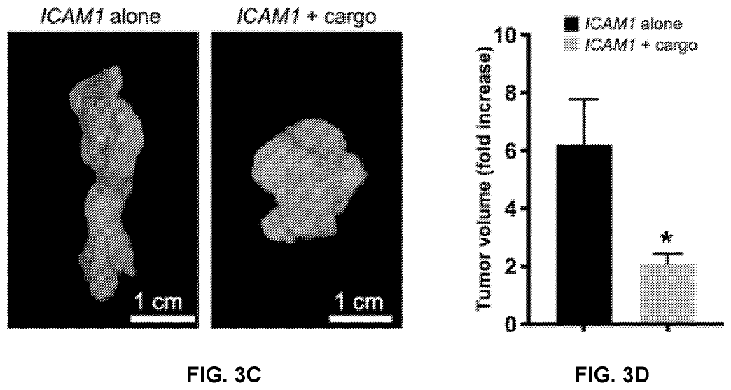

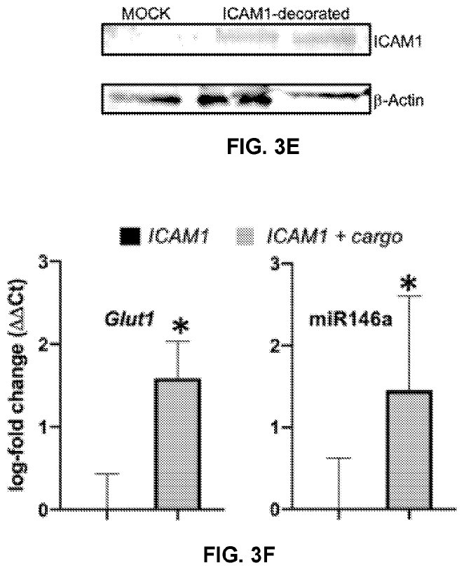

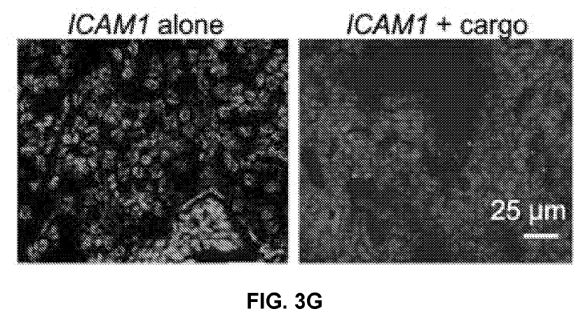

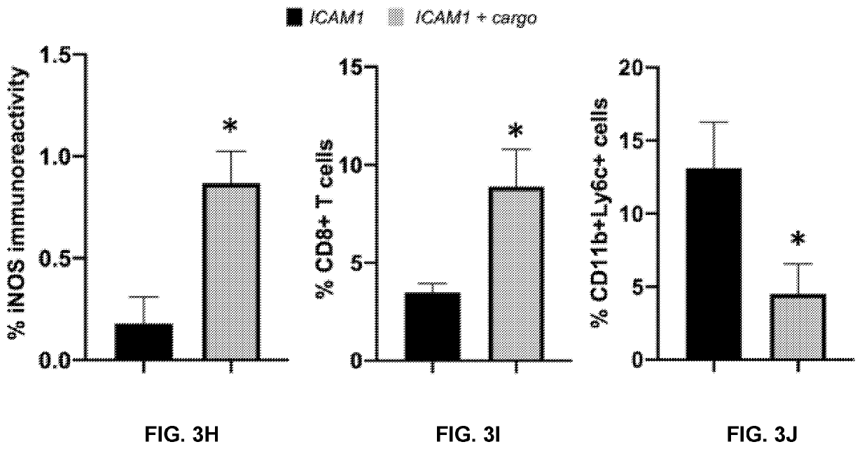

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software.

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

### Compositions

Disclosed herein are vaccine compositions that involves a first polynucleotide encoding or comprising a viral, bacterial, or tumor antigen, and a second polynucleotide encoding a fusion protein comprising an APC-targeting ligand and an exosomal or lysosomal transmembrane protein. Also disclosed is a method of vaccinating a subject that involves transfecting skin cells of the subject with the disclosed vaccine composition. As disclosed herein, this method will cause skin-resident skin cells to produce EVs containing the viral, bacterial, or tumor antigen and decorated on the surface with an APC-targeting ligand.

The disclosed EVs can in some embodiments be any vesicle that can be secreted by a cell, such as a skin cell. Cells secrete extracellular vesicles (EVs) with a broad range of diameters and functions, including apoptotic bodies (1-5 μm), microvesicles (100-1000 nm in size), and vesicles of endosomal origin, known as exosomes (50-150 nm).

### Viral Antigen

Also disclosed herein are polynucleotides comprising nucleic acid sequences encoding or comprising a viral, bacterial, or tumor antigen, such as those known in the art for RNA or DNA vaccines.

In some embodiments, the viral antigen is mRNA-1273.

In some embodiments, the viral antigen is SARS-COV2 spike protein. Therefore, in some embodiments, the first polynucleotide encodes a viral antigen having the amino acid sequence: MFVFLVLLPLVSSQCVNLTTRTQLPPAYTNSFTRGVYYPDKVFRSSVLHSTQDLFLPFF SNVTWFHAIHVSGTNGTKRFDNPVLPFNDGVYFASTEKSNIIRGWIFGTTLDSKTQSLLI VNNATNVVIKVCEFQFCNDPFLGVYYHKNNKSWMESEFRVYSSANNCTFEYVSQPFL MDLEGKQGNFKNLREFVFKNIDGYFKIYSKHTPINLVRDLPQGFSALEPLVDLPIGINITR FQTLLALHRSYLTPGDSSSGWTAGAAAYYVGYLQPRTFLLKYNENGTITDAVDCALDPL SETKCTLKSFTVEKGIYQTSNFRVQPTESIVRFPNITNLCPFGEVFNATRFASVYAWNRK RISNCVADYSVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFERDISTEIYQ AGSTPCNGVEGFNCYFPLQSYGFQPTNGVGYQPYRVVVLSFELLHAPATVCGPKKST NLVKNKCVNFNFNGLTGTGVLTESNKKFLPFQQFGRDIADTTDAVRDPQTLEILDITPCS FGGVSVITPGTNTSNQVAVLYQDVNCTEVPVAIHADQLTPTWRVYSTGSNVFQTRAGC LIGAEHVNNSYECDIPIGAGICASYQTQTNSPRRARSVASQSIIAYTMSLGAENSVAYSN NSIAIPTNFTISVTTEILPVSMTKTSVDCTMYICGDSTECSNLLLQYGSFCTQLNRALTGIA VEQDKNTQEVFAQVKQIYKTPPIKDFGGFNFSQILPDPSKPSKRSFIEDLLFNKVTLADA GFIKQYGDCLGDIAARDLICAQKFNGLTVLPPLLTDEMIAQYTSALLAGTITSGWTFGAG AALQIPFAMQMAYRFNGIGVTQNVLYENQKLIANQFNSAIGKIQDSLSSTASALGKLQDV VNQNAQALNTLVKQLSSNFGAISSVLNDILSRLDKVEAEVQIDRLITGRLQSLQTYVTQQ LIRAAEIRASANLAATKMSECVLGQSKRVDFCGKGYHLMSFPQSAPHGVVFLHVTYVP AQEKNFTTAPAICHDGKAHFPREGVFVSNGTHWFVTQRNFYEPQIITTDNTFVSGNCD VVIGIVNNTVYDPLQPELDSFKEELDKYFKNHTSPDVDLGDISGINASVVNIQKEIDRLNE VAKNLNESLIDLQELGKYEQYIKWPWYIWLGFIAGLIAIVMVTIMLCCMTSCCSCLKGCC SCGSCCKFDEDDSEPVLKGVKLHYT (SEQ ID NO:1, YP_009724390), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1. Therefore, in some embodiments, the first polynucleotide has the nucleic acid sequence: ATGTTTGTTTTTCTTGTTTTATTGCCACTAGTCTCTAGTCAGTGTGTTAATCTTACAA CCAGAACTCAATTACCCCCTGCATACACTAATTCTTTCACACGTGGTGTTTATTACC CTGACAAAGTTTTCAGATCCTCAGTTTTACATTCAACTCAGGACTTGTTCTTACCTTT CTTTTCCAATGTTACTTGGTTCCATGCTATACATGTCTCTGGGACCAATGGTACTAA GAGGTTTGATAACCCTGTCCTACCATTTAATGATGGTGTTTATTTTGCTTCCACTGA GAAGTCTAACATAATAAGAGGCTGGATTTTTGGTACTACTTTAGATTCGAAGACCCA GTCCCTACTTATTGTTAATAACGCTACTAATGTTGTTATTAAAGTCTGTGAATTTCAA TTTTGTAATGATCCATTTTTGGGTGTTTATTACCACAAAAACAACAAAAGTTGGATGG AAAGTGAGTTCAGAGTTTATTCTAGTGCGAATAATTGCACTTTTGAATATGTCTCTCA GCCTTTTCTTATGGACCTTGAAGGAAAACAGGGTAATTTCAAAAATCTTAGGGAATT TGTGTTTAAGAATATTGATGGTTATTTTAAAATATATTCTAAGCACACGCCTATTAATT TAGTGCGTGATCTCCCTCAGGGTTTTTCGGCTTTAGAACCATTGGTAGATTTGCCAA TAGGTATTAACATCACTAGGTTTCAAACTTTACTTGCTTTACATAGAAGTTATTTGAC TCCTGGTGATTCTTCTTCAGGTTGGACAGCTGGTGCTGCAGCTTATTATGTGGGTTA TCTTCAACCTAGGACTTTTCTATTAAAATATAATGAAAATGGAACCATTACAGATGCT GTAGACTGTGCACTTGACCCTCTCTCAGAAACAAAGTGTACGTTGAAATCCTTCACT GTAGAAAAAGGAATCTATCAAACTTCTAACTTTAGAGTCCAACCAACAGAATCTATT GTTAGATTTCCTAATATTACAAACTTGTGCCCTTTTGGTGAAGTTTTTAACGCCACCA GATTTGCATCTGTTTATGCTTGGAACAGGAAGAGAATCAGCAACTGTGTTGCTGATT ATTCTGTCCTATATAATTCCGCATCATTTTCCACTTTTAAGTGTTATGGAGTGTCTCC TACTAAATTAAATGATCTCTGCTTTACTAATGTCTATGCAGATTCATTTGTAATTAGA GGTGATGAAGTCAGACAAATCGCTCCAGGGCAAACTGGAAAGATTGCTGATTATAA TTATAAATTACCAGATGATTTTACAGGCTGCGTTATAGCTTGGAATTCTAACAATCTT GATTCTAAGGTTGGTGGTAATTATAATTACCTGTATAGATTGTTTAGGAAGTCTAATC TCAAACCTTTTGAGAGAGATATTTCAACTGAAATCTATCAGGCCGGTAGCACACCTT GTAATGGTGTTGAAGGTTTTAATTGTTACTTTCCTTTACAATCATATGGTTTCCAACC CACTAATGGTGTTGGTTACCAACCATACAGAGTAGTAGTACTTTCTTTTGAACTTCTA CATGCACCAGCAACTGTTTGTGGACCTAAAAAGTCTACTAATTTGGTTAAAAACAAA TGTGTCAATTTCAACTTCAATGGTTTAACAGGCACAGGTGTTCTTACTGAGTCTAAC AAAAAGTTTCTGCCTTTCCAACAATTTGGCAGAGACATTGCTGACACTACTGATGCT GTCCGTGATCCACAGACACTTGAGATTCTTGACATTACACCATGTTCTTTTGGTGGT GTCAGTGTTATAACACCAGGAACAAATACTTCTAACCAGGTTGCTGTTCTTTATCAG GATGTTAACTGCACAGAAGTCCCTGTTGCTATTCATGCAGATCAACTTACTCCTACT TGGCGTGTTTATTCTACAGGTTCTAATGTTTTTCAAACACGTGCAGGCTGTTTAATA GGGGCTGAACATGTCAACAACTCATATGAGTGTGACATACCCATTGGTGCAGGTAT ATGCGCTAGTTATCAGACTCAGACTAATTCTCCTCGGCGGGCACGTAGTGTAGCTA GTCAATCCATCATTGCCTACACTATGTCACTTGGTGCAGAAAATTCAGTTGCTTACT CTAATAACTCTATTGCCATACCCACAAATTTTACTATTAGTGTTACCACAGAAATTCT ACCAGTGTCTATGACCAAGACATCAGTAGATTGTACAATGTACATTTGTGGTGATTC AACTGAATGCAGCAATCTTTTGTTGCAATATGGCAGTTTTTGTACACAATTAAACCGT GCTTTAACTGGAATAGCTGTTGAACAAGACAAAAACACCCAAGAAGTTTTTGCACAA GTCAAACAAATTTACAAAACACCACCAATTAAAGATTTTGGTGGTTTTAATTTTTCAC AAATATTACCAGATCCATCAAAACCAAGCAAGAGGTCATTTATTGAAGATCTACTTTT CAACAAAGTGACACTTGCAGATGCTGGCTTCATCAAACAATATGGTGATTGCCTTG GTGATATTGCTGCTAGAGACCTCATTTGTGCACAAAAGTTTAACGGCCTTACTGTTT TGCCACCTTTGCTCACAGATGAAATGATTGCTCAATACACTTCTGCACTGTTAGCGG GTACAATCACTTCTGGTTGGACCTTTGGTGCAGGTGCTGCATTACAAATACCATTTG CTATGCAAATGGCTTATAGGTTTAATGGTATTGGAGTTACACAGAATGTTCTCTATG AGAACCAAAAATTGATTGCCAACCAATTTAATAGTGCTATTGGCAAAATTCAAGACT CACTTTCTTCCACAGCAAGTGCACTTGGAAAACTTCAAGATGTGGTCAACCAAAATG CACAAGCTTTAAACACGCTTGTTAAACAACTTAGCTCCAATTTTGGTGCAATTTCAA GTGTTTTAAATGATATCCTTTCACGTCTTGACAAAGTTGAGGCTGAAGTGCAAATTG ATAGGTTGATCACAGGCAGACTTCAAAGTTTGCAGACATATGTGACTCAACAATTAA TTAGAGCTGCAGAAATCAGAGCTTCTGCTAATCTTGCTGCTACTAAAATGTCAGAGT GTGTACTTGGACAATCAAAAAGAGTTGATTTTTGTGGAAAGGGCTATCATCTTATGT CCTTCCCTCAGTCAGCACCTCATGGTGTAGTCTTCTTGCATGTGACTTATGTCCCTG CACAAGAAAAGAACTTCACAACTGCTCCTGCCATTTGTCATGATGGAAAAGCACACT TTCCTCGTGAAGGTGTCTTTGTTTCAAATGGCACACACTGGTTTGTAACACAAAGGA ATTTTTATGAACCACAAATCATTACTACAGACAACACATTTGTGTCTGGTAACTGTGA TGTTGTAATAGGAATTGTCAACAACACAGTTTATGATCCTTTGCAACCTGAATTAGA CTCATTCAAGGAGGAGTTAGATAAATATTTTAAGAATCATACATCACCAGATGTTGAT TTAGGTGACATCTCTGGCATTAATGCTTCAGTTGTAAACATTCAAAAAGAAATTGAC CGCCTCAATGAGGTTGCCAAGAATTTAAATGAATCTCTCATCGATCTCCAAGAACTT GGAAAGTATGAGCAGTATATAAAATGGCCATGGTACATTTGGCTAGGTTTTATAGCT GGCTTGATTGCCATAGTAATGGTGACAATTATGCTTTGCTGTATGACCAGTTGCTGT AGTTGTCTCAAGGGCTGTTGTTCTTGTGGATCCTGCTGCAAATTTGATGAAGACGA CTCTGAGCCAGTGCTCAAAGGAGTCAAATTACATTACACC (SEQ ID NO:2, NC_045512.2), or a nucleic acid sequence that hybridizes to a nucleic acid sequence consisting of SEQ ID NO:2 under stringent hybridization conditions.

In some embodiments, the tumor antigen is a Her-2/neu protein. Therefore, in some embodiments, the first polynucleotide encodes a viral antigen having the amino acid sequence: KGRPWPLWSGPRAAARPFPRGPLLRRAPGPHPSQHPAPRALPAGSSRSHGAGAAVS TMELAALCRWGLLLALLPPGAASTQVCTGTDMKLRLPASPETHLDMLRHLYQGCQVV QGNLELTYLPTNASLSFLQDIQEVQGYVLIAHNQVRQVPLQRLRIVRGTQLFEDNYALA VLDNGDPLNNTTPVTGASPGGLRELQLRSLTEILKGGVLIQRNPQLCYQDTILWKDIFHK NNQLALTLIDTNRSRACHPCSPMCKGSRCWGESSEDCQSLTRTVCAGGCARCKGPLP TDCCHEQCAAGCTGPKHSDCLACLHFNHSGICELHCPALVTYNTDTFESMPNPEGRYT FGASCVTACPYNYLSTDVGSCTLVCPLHNQEVTAEDGTQRCEKCSKPCARVCYGLGM EHLREVRAVTSANIQEFAGCKKIFGSLAFLPESFDGDPASNTAPLQPEQLQVFETLEEIT GYLYISAWPDSLPDLSVFQNLQVIRGRILHNGAYSLTLQGLGISWLGLRSLRELGSGLAL IHHNTHLCFVHTVPWDQLFRNPHQALLHTANRPEDECVGEGLACHQLCARGHCWGP GPTQCVNCSQFLRGQECVEECRVLQGLPREYVNARHCLPCHPECQPQNGSVTCFGP EADQCVACAHYKDPPFCVARCPSGVKPDLSYMPIWKFPDEEGACQPCPINCTHSCVD LDDKGCPAEQRASPLTSIISAVVGILLVVVLGVVFGILIKRRQQKIRKYTMRRLLQETELV EPLTPSGAMPNQAQMRILKETELRKVKVLGSGAFGTVYKG WIPDGENVKIPVAIKVLRE NTSPKANKEILDEAYVMAGVGSPYVSRLLGICLTSTVQLVTQLMPYGCLLDHVRENRGR LGSQDLLNWCMQIAKGMSYLEDVRLVHRDLAARNVLVKSPNHVKITDFGLARLLDIDET EYHADGGKVPIKWMALESILRRRFTHQSDVWSYGVTVWELMTFGAKPYDGIPAREIPD LLEKGERLPQPPICTIDVYMIMVKCWMIDSECRPRFRELVSEFSRMARDPQRFVVIQNE DLGPASPLDSTFYRSLLEDDDMGDLVDAEEYLVPQQGFFCPDPAPGAGGMVHHRHRS SSTRSGGGDLTLGLEPSEEEAPRSPLAPSEGAGSDVFDGDLGMGAAKGLQSLPTHDP SPLQRYSEDPTVPLPSETDGYVAPLTCSPQPEYVNQPDVRPQPPSPREGPLPAARPA GATLERPKTLSPGKNGVVKDVFAFGGAVENPEYLTPQGGAAPQPHPPPAFSPAFDNLY YWDQDPPERGAPPSTFKGTPTAENPEYLGLDVPVTRRPSPQKPCVLREQGRPDFCW HQEVGGPSDHFQGNLPCQEPVLRNLPSCLSSQMAGRGPASLEEEQHWGVFVDSEAL PNETLGSSGCHSPAWPFPSRSWVLKALGKLAEGKRPGSVEQKRPIQRLSLKPSTAPHE EGTAMVSVSRLCTECFSVFLLFLFCFFKDEIKTQGE (SEQ ID NO:37), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:37. Therefore, in some embodiments, the first polynucleotide has the nucleic acid sequence: AAGGGGAGGTAACCCTGGCCCCTTTGGTCGGGGCCCCGGGCAGCCGCGCGCCCC TTCCCACGGGGCCCTTTACTGCGCCGCGCGCCCGGCCCCCACCCCTCGCAGCAC CCCGCGCCCCGCGCCCTCCCAGCCGGGTCCAGCCGGAGCCATGGGGCCGGAGC CGCAGTGAGCACCATGGAGCTGGCGGCCTTGTGCCGCTGGGGGCTCCTCCTCGC CCTCTTGCCCCCCGGAGCCGCGAGCACCCAAGTGTGCACCGGCACAGACATGAAG CTGCGGCTCCCTGCCAGTCCCGAGACCCACCTGGACATGCTCCGCCACCTCTACC AGGGCTGCCAGGTGGTGCAGGGAAACCTGGAACTCACCTACCTGCCCACCAATGC CAGCCTGTCCTTCCTGCAGGATATCCAGGAGGTGCAGGGCTACGTGCTCATCGCT CACAACCAAGTGAGGCAGGTCCCACTGCAGAGGCTGCGGATTGTGCGAGGCACCC AGCTCTTTGAGGACAACTATGCCCTGGCCGTGCTAGACAATGGAGACCCGCTGAA CAATACCACCCCTGTCACAGGGGCCTCCCCAGGAGGCCTGCGGGAGCTGCAGCTT CGAAGCCTCACAGAGATCTTGAAAGGAGGGGTCTTGATCCAGCGGAACCCCCAGC TCTGCTACCAGGACACGATTTTGTGGAAGGACATCTTCCACAAGAACAACCAGCTG GCTCTCACACTGATAGACACCAACCGCTCTCGGGCCTGCCACCCCTGTTCTCCGAT GTGTAAGGGCTCCCGCTGCTGGGGAGAGAGTTCTGAGGATTGTCAGAGCCTGACG CGCACTGTCTGTGCCGGTGGCTGTGCCCGCTGCAAGGGGCCACTGCCCACTGACT GCTGCCATGAGCAGTGTGCTGCCGGCTGCACGGGCCCCAAGCACTCTGACTGCCT GGCCTGCCTCCACTTCAACCACAGTGGCATCTGTGAGCTGCACTGCCCAGCCCTG GTCACCTACAACACAGACACGTTTGAGTCCATGCCCAATCCCGAGGGCCGGTATAC ATTCGGCGCCAGCTGTGTGACTGCCTGTCCCTACAACTACCTTTCTACGGACGTGG GATCCTGCACCCTCGTCTGCCCCCTGCACAACCAAGAGGTGACAGCAGAGGATGG AACACAGCGGTGTGAGAAGTGCAGCAAGCCCTGTGCCCGAGTGTGCTATGGTCTG GGCATGGAGCACTTGCGAGAGGTGAGGGCAGTTACCAGTGCCAATATCCAGGAGT TTGCTGGCTGCAAGAAGATCTTTGGGAGCCTGGCATTTCTGCCGGAGAGCTTTGAT GGGGACCCAGCCTCCAACACTGCCCCGCTCCAGCCAGAGCAGCTCCAAGTGTTTG AGACTCTGGAAGAGATCACAGGTTACCTATACATCTCAGCATGGCCGGACAGCCTG CCTGACCTCAGCGTCTTCCAGAACCTGCAAGTAATCCGGGGACGAATTCTGCACAA TGGCGCCTACTCGCTGACCCTGCAAGGGCTGGGCATCAGCTGGCTGGGGCTGCG CTCACTGAGGGAACTGGGCAGTGGACTGGCCCTCATCCACCATAACACCCACCTC TGCTTCGTGCACACGGTGCCCTGGGACCAGCTCTTTCGGAACCCGCACCAAGCTC TGCTCCACACTGCCAACCGGCCAGAGGACGAGTGTGTGGGCGAGGGCCTGGCCT GCCACCAGCTGTGCGCCCGAGGGCACTGCTGGGGTCCAGGGCCCACCCAGTGTG TCAACTGCAGCCAGTTCCTTCGGGGCCAGGAGTGCGTGGAGGAATGCCGAGTACT GCAGGGGCTCCCCAGGGAGTATGTGAATGCCAGGCACTGTTTGCCGTGCCACCCT GAGTGTCAGCCCCAGAATGGCTCAGTGACCTGTTTTGGACCGGAGGCTGACCAGT GTGTGGCCTGTGCCCACTATAAGGACCCTCCCTTCTGCGTGGCCCGCTGCCCCAG CGGTGTGAAACCTGACCTCTCCTACATGCCCATCTGGAAGTTTCCAGATGAGGAGG GCGCATGCCAGCCTTGCCCCATCAACTGCACCCACTCCTGTGTGGACCTGGATGA CAAGGGCTGCCCCGCCGAGCAGAGAGCCAGCCCTCTGACGTCCATCATCTCTGCG GTGGTTGGCATTCTGCTGGTCGTGGTCTTGGGGGTGGTCTTTGGGATCCTCATCAA GCGACGGCAGCAGAAGATCCGGAAGTACACGATGCGGAGACTGCTGCAGGAAAC GGAGCTGGTGGAGCCGCTGACACCTAGCGGAGCGATGCCCAACCAGGCGCAGAT GCGGATCCTGAAAGAGACGGAGCTGAGGAAGGTGAAGGTGCTTGGATCTGGCGCT TTTGGCACAGTCTACAAGGGCATCTGGATCCCTGATGGGGAGAATGTGAAAATTCC AGTGGCCATCAAAGTGTTGAGGGAAAACACATCCCCCAAAGCCAACAAAGAAATCT TAGACGAAGCATACGTGATGGCTGGTGTGGGCTCCCCATATGTCTCCCGCCTTCTG GGCATCTGCCTGACATCCACGGTGCAGCTGGTGACACAGCTTATGCCCTATGGCT GCCTCTTAGACCATGTCCGGGAAAACCGCGGACGCCTGGGCTCCCAGGACCTGCT GAACTGGTGTATGCAGATTGCCAAGGGGATGAGCTACCTGGAGGATGTGCGGCTC GTACACAGGGACTTGGCCGCTCGGAACGTGCTGGTCAAGAGTCCCAACCATGTCA AAATTACAGACTTCGGGCTGGCTCGGCTGCTGGACATTGACGAGACAGAGTACCAT GCAGATGGGGGCAAGGTGCCCATCAAGTGGATGGCGCTGGAGTCCATTCTCCGCC GGCGGTTCACCCACCAGAGTGATGTGTGGAGTTATGGTGTGACTGTGTGGGAGCT GATGACTTTTGGGGCCAAACCTTACGATGGGATCCCAGCCCGGGAGATCCCTGAC CTGCTGGAAAAGGGGGAGCGGCTGCCCCAGCCCCCCATCTGCACCATTGATGTCT ACATGATCATGGTCAAATGTTGGATGATTGACTCTGAATGTCGGCCAAGATTCCGG GAGTTGGTGTCTGAATTCTCCCGCATGGCCAGGGACCCCCAGCGCTTTGTGGTCA TCCAGAATGAGGACTTGGGCCCAGCCAGTCCCTTGGACAGCACCTTCTACCGCTC ACTGCTGGAGGACGATGACATGGGGGACCTGGTGGATGCTGAGGAGTATCTGGTA CCCCAGCAGGGCTTCTTCTGTCCAGACCCTGCCCCGGGCGCTGGGGGCATGGTC CACCACAGGCACCGCAGCTCATCTACCAGGAGTGGCGGTGGGGACCTGACACTAG GGCTGGAGCCCTCTGAAGAGGAGGCCCCCAGGTCTCCACTGGCACCCTCCGAAG GGGCTGGCTCCGATGTATTTGATGGTGACCTGGGAATGGGGGCAGCCAAGGGGCT GCAAAGCCTCCCCACACATGACCCCAGCCCTCTACAGCGGTACAGTGAGGACCCC ACAGTACCCCTGCCCTCTGAGACTGATGGCTACGTTGCCCCCCTGACCTGCAGCC CCCAGCCTGAATATGTGAACCAGCCAGATGTTCGGCCCCAGCCCCCTTCGCCCCG AGAGGGCCCTCTGCCTGCTGCCCGACCTGCTGGTGCCACTCTGGAAAGGCCCAAG ACTCTCTCCCCAGGGAAGAATGGGGTCGTCAAAGACGTTTTTGCCTTTGGGGGTGC CGTGGAGAACCCCGAGTACTTGACACCCCAGGGAGGAGCTGCCCCTCAGCCCCAC CCTCCTCCTGCCTTCAGCCCAGCCTTCGACAACCTCTATTACTGGGACCAGGACCC ACCAGAGCGGGGGGCTCCACCCAGCACCTTCAAAGGGACACCTACGGCAGAGAA CCCAGAGTACCTGGGTCTGGACGTGCCAGTGTGAACCAGAAGGCCAAGTCCGCAG AAGCCCTGATGTGTCCTCAGGGAGCAGGGAAGGCCTGACTTCTGCTGGCATCAAG AGGTGGGAGGGCCCTCCGACCACTTCCAGGGGAACCTGCCATGCCAGGAACCTGT CCTAAGGAACCTTCCTTCCTGCTTGAGTTCCCAGATGGCTGGAAGGGGTCCAGCCT CGTTGGAAGAGGAACAGCACTGGGGAGTCTTTGTGGATTCTGAGGCCCTGCCCAA TGAGACTCTAGGGTCCAGTGGATGCCACAGCCCAGCTTGGCCCTTTCCTTCCAGAT CCTGGGTACTGAAAGCCTTAGGGAAGCTGGCCTGAGAGGGGAAGCGGCCCTAAG GGAGTGTCTAAGAACAAAAGCGACCCATTCAGAGACTGTCCCTGAAACCTAGTACT GCCCCCCATGAGGAAGGAACAGCAATGGTGTCAGTATCCAGGCTTTGTACAGAGT GCTTTTCTGTTTAGTTTTTACTTTTTTTGTTTTGTTTTTTTAAAGATGAAATAAAGACC CAGGGGGAG (SEQ ID NO:38), or a nucleic acid sequence that hybridizes to a nucleic acid sequence consisting of SEQ ID NO:38 under stringent hybridization conditions.

In some embodiments, the bacterial antigen is a Staphylococcus aureus protein. Therefore, in some embodiments, the first polynucleotide encodes a viral antigen having the amino acid sequence: MKLFAFIFICVKSCSLLFMLNGNPRPEQLNKASEFTGLMDNMRYLYDDKHVSETNIKAQ EKFLQHDLLFKINGSKIDGSKILKTEFNNKSLSDKYKNKNVDLFGTNYYNQCYFSADNM ELNDGRLIEKTCMYGGVTEHDGNQIDKNNLTDNSHNILIKVYENERNTLSFDISTNKKNIT AQEIDYKVRNYLLKHKNLYKFNSSPYETGYIKFIEGNGHSFWYDMMPESGEKFYPTKYL LIYNDNKTVESKSINVEVHLTKK (SEQ ID NO:39), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:39. Therefore, in some embodiments, the first polynucleotide has the nucleic acid sequence: ATGAAGTTATTTGCTTTTATCTTCATATGTGTTAAGTCTTGCAGCTTACTATTTATGTT AAATGGCAATCCTAGACCAGAACAATTGAATAAAGCGAGTGAATTCACTGGTCTAAT GGATAATATGAGGTATTTGTATGACGATAAACACGTATCAGAAACAAACATTAAAGC CCAAGAGAAGTTTTTACAACATGATTTATTATTTAAAATAAATGGCTCTAAAATTGAT GGTTCTAAAATTTTAAAAACAGAATTTAATAATAAAAGCCTTTCGGATAAATACAAAA ATAAAAACGTAGATTTGTTTGGGACAAACTATTATAATCAATGCTATTTTTCAGCGGA TAATATGGAATTAAATGATGGTAGACTAATTGAAAAAACGTGTATGTATGGCGGTGT GACCGAGCATGATGGAAATCAAATAGATAAAAATAATCTAACTGATAACTCTCATAA TATCTTAATTAAAGTATACGAAAACGAAAGAAACACATTATCTTTTGATATATCTACC AATAAGAAAAACATAACAGCACAAGAAATAGATTATAAAGTTAGAAACTATTTACTTA AGCATAAAAATTTATATAAATTTAATAGTTCGCCTTATGAGACTGGCTATATAAAGTT TATCGAAGGAAATGGTCATTCTTTTTGGTATGATATGATGCCTGAATCTGGTGAAAA ATTTTATCCGACTAAATATTTACTAATTTATAATGATAATAAGACAGTTGAGAGTAAAT CTATTAATGTAGAAGTTCATTTAACCAAAAAATAA (SEQ ID NO:40), or a nucleic acid sequence that hybridizes to a nucleic acid sequence consisting of SEQ ID NO:40 under stringent hybridization conditions.

### APC-Targeting Ligand

Also disclosed are polynucleotides comprising nucleic acid sequences encoding an APC-targeting ligand. In some embodiments, the APC-targeting ligand comprises ICAM1 or ICAM4. In some embodiments, the APC-targeting ligand is selected from the group consisting of CD2, CD11a, CD18, CD22, CD29, CD40L, LDL, oxLDL, Lectins, Galectin 1, Galectin 3, Flagellin, Cxcl5, KRT14, FGF7, FGF10, and AMP-IBP5.

In some embodiments, the APC-targeting ligand comprises ICAM1 or ICAM4. Therefore, in some embodiments, the APC-targeting ligand is ICAM1 and comprises the amino acid sequence: ELLCYSELQPQPRYGSQQPPARAARTPGPARGSVPRTWQCPDICVPLKSHPAPGRLR AGDMQHLLPAQVVGHRDPVAKGVAPAWEQPEGVTEQCARRPTNVLFKLPWAVNSNL PHRVLDSRTGGTGTPPLLAASGQEPYPTLPGGGWGTPGQPHRGAAPWGEGAETGAS CGGARGHDHGAGEERSPWSQFLVPHTGPAAPRAGAVEHLGPLPAPDLCPASDSPTT CQPPGPRGGHAGDRGLFPGRAVPSLGGPGPPGTGGPEVEPHSHLWQRLLLGQGLSQ CDRRGRGHPAADVCSNTGEPEPGDTADSDHLQLSGAQRDSDEARGLRRDRGDSEVG PPSQGDAEWGSSPATGPEGPAPAEGH PRGQRAQLLLLCNPGGGRPAYTQEPDPGAS CPVWPPTGREGLSGKLDVARKFPADSNVPGLGEPIARAQVSKGWH FPTAH RGISDCH SRSGHLPLSGQEHSRGGHPQGDRECALPPVDCHHHCGSSRSHNGHCRPQHVPLPPA EDQEIQTTTGPKRDPHETEHTSHASLNLSRDRASSSAFPYWWQWCHTEQSGRHMPC SYTYRPWDAGGQGIVLSQIQQHLGPWYLHTNTRPRISVVTLSQEEGARLKTLMDVKVP DERGSGGGDIAPPGHTTGKYNLLPIGYAEAPQTYRRSGPPTCVASKHKGPHFLTDASL GTAVYPQPLMICIYSFVILPAIYVSFMAKTVSGLTELPVLITFKVTRYSCTGCTLQESAWQ KDQMGLGLLIGQPAFPQKEFFYRHKSTIWTGNGYRFRDYPVRPYSSLPPKTDTFVSHL PTHIHFCQCSQHSAVMSGHECPGNMPKLCLVLLSCLHFTGSLHYAAPVSCSDQGPAS SGEGGQGIGGLPPSFGSLIRVCVCVYVTSSRSVTQAGVQWCNHGSLQSPFGLKSSHL SLLSSWDHRLTTPHLANLIFFFFPETGSRNIAQTSFVLVNKAFSTA (SEQ ID NO:3, NM_000201.3), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:3. Therefore, in some embodiments, the polynucleotide encoding the encoding the APC-targeting ligand has the nucleic acid sequence: GAGCTCCTCTGCTACTCAGAGTTGCAACCTCAGCCTCGCTATGGCTCCCAGCAGCC CCCGGCCCGCGCTGCCCGCACTCCTGGTCCTGCTCGGGGCTCTGTTCCCAGGAC CTGGCAATGCCCAGACATCTGTGTCCCCCTCAAAAGTCATCCTGCCCCGGGGAGG CTCCGTGCTGGTGACATGCAGCACCTCCTGTGACCAGCCCAAGTTGTTGGGCATA GAGACCCCGTTGCCTAAAAAGGAGTTGCTCCTGCCTGGGAACAACCGGAAGGTGT ATGAACTGAGCAATGTGCAAGAAGATAGCCAACCAATGTGCTATTCAAACTGCCCT GATGGGCAGTCAACAGCTAAAACCTTCCTCACCGTGTACTGGACTCCAGAACGGGT GGAACTGGCACCCCTCCCCTCTTGGCAGCCAGTGGGCAAGAACCTTACCCTACGC TGCCAGGTGGAGGGTGGGGCACCCCGGGCCAACCTCACCGTGGTGCTGCTCCGT GGGGAGAAGGAGCTGAAACGGGAGCCAGCTGTGGGGGAGCCCGCTGAGGTCACG ACCACGGTGCTGGTGAGGAGAGATCACCATGGAGCCAATTTCTCGTGCCGCACTG AACTGGACCTGCGGCCCCAAGGGCTGGAGCTGTTTGAGAACACCTCGGCCCCCTA CCAGCTCCAGACCTTTGTCCTGCCAGCGACTCCCCCACAACTTGTCAGCCCCCGG GTCCTAGAGGTGGACACGCAGGGGACCGTGGTCTGTTCCCTGGACGGGCTGTTCC CAGTCTCGGAGGCCCAGGTCCACCTGGCACTGGGGGACCAGAGGTTGAACCCCA CAGTCACCTATGGCAACGACTCCTTCTCGGCCAAGGCCTCAGTCAGTGTGACCGC AGAGGACGAGGGCACCCAGCGGCTGACGTGTGCAGTAATACTGGGGAACCAGAG CCAGGAGACACTGCAGACAGTGACCATCTACAGCTTTCCGGCGCCCAACGTGATT CTGACGAAGCCAGAGGTCTCAGAAGGGACCGAGGTGACAGTGAAGTGTGAGGCC CACCCTAGAGCCAAGGTGACGCTGAATGGGGTTCCAGCCCAGCCACTGGGCCCGA GGGCCCAGCTCCTGCTGAAGGCCACCCCAGAGGACAACGGGCGCAGCTTCTCCT GCTCTGCAACCCTGGAGGTGGCCGGCCAGCTTATACACAAGAACCAGACCCGGGA GCTTCGTGTCCTGTATGGCCCCCGACTGGACGAGAGGGATTGTCCGGGAAACTGG ACGTGGCCAGAAAATTCCCAGCAGACTCCAATGTGCCAGGCTTGGGGGAACCCAT TGCCCGAGCTCAAGTGTCTAAAGGATGGCACTTTCCCACTGCCCATCGGGGAATCA GTGACTGTCACTCGAGATCTTGAGGGCACCTACCTCTGTCGGGCCAGGAGCACTC AAGGGGAGGTCACCCGCAAGGTGACCGTGAATGTGCTCTCCCCCCGGTATGAGAT TGTCATCATCACTGTGGTAGCAGCCGCAGTCATAATGGGCACTGCAGGCCTCAGCA CGTACCTCTATAACCGCCAGCGGAAGATCAAGAAATACAGACTACAACAGGCCCAA AAAGGGACCCCCATGAAACCGAACACACAAGCCACGCCTCCCTGAACCTATCCCG GGACAGGGCCTCTTCCTCGGCCTTCCCATATTGGTGGCAGTGGTGCCACACTGAA CAGAGTGGAAGACATATGCCATGCAGCTACACCTACCGGCCCTGGGACGCCGGAG GACAGGGCATTGTCCTCAGTCAGATACAACAGCATTTGGGGCCATGGTACCTGCAC ACCTAAAACACTAGGCCACGCATCTGATCTGTAGTCACATGACTAAGCCAAGAGGA AGGAGCAAGACTCAAGACATGATTGATGGATGTTAAAGTCTAGCCTGATGAGAGGG GAAGTGGTGGGGGAGACATAGCCCCACCATGAGGACATACAACTGGGAAATACTG AAACTTGCTGCCTATTGGGTATGCTGAGGCCCCACAGACTTACAGAAGAAGTGGCC CTCCATAGACATGTGTAGCATCAAAACACAAAGGCCCACACTTCCTGACGGATGCC AGCTTGGGCACTGCTGTCTACTGACCCCAACCCTTGATGATATGTATTTATTCATTT GTTATTTTACCAGCTATTTATTGAGTGTCTTTTATGTAGGCTAAATGAACATAGGTCT CTGGCCTCACGGAGCTCCCAGTCCTAATCACATTCAAGGTCACCAGGTACAGTTGT ACAGGTTGTACACTGCAGGAGAGTGCCTGGCAAAAAGATCAAATGGGGCTGGGAC TTCTCATTGGCCAACCTGCCTTTCCCCAGAAGGAGTGATTTTTCTATCGGCACAAAA GCACTATATGGACTGGTAATGGTTACAGGTTCAGAGATTACCCAGTGAGGCCTTAT TCCTCCCTTCCCCCCAAAACTGACACCTTTGTTAGCCACCTCCCCACCCACATACAT TTCTGCCAGTGTTCACAATGACACTCAGCGGTCATGTCTGGACATGAGTGCCCAGG GAATATGCCCAAGCTATGCCTTGTCCTCTTGTCCTGTTTGCATTTCACTGGGAGCTT GCACTATGCAGCTCCAGTTTCCTGCAGTGATCAGGGTCCTGCAAGCAGTGGGGAA GGGGGCCAAGGTATTGGAGGACTCCCTCCCAGCTTTGGAAGCCTCATCCGCGTGT GTGTGTGTGTGTATGTGTAGACAAGCTCTCGCTCTGTCACCCAGGCTGGAGTGCAG TGGTGCAATCATGGTTCACTGCAGTCTTGACCTTTTGGGCTCAAGTGATCCTCCCA CCTCAGCCTCCTGAGTAGCTGGGACCATAGGCTCACAACACCACACCTGGCAAATT TGATTTTTTTTTTTTTTCCAGAGACGGGGTCTCGCAACATTGCCCAGACTTCCTTTGT GTTAGTTAATAAAGCTTTCTCAACTGCC (SEQ ID NO:4), or a nucleic acid sequence that hybridizes to a nucleic acid sequence consisting of SEQ ID NO:4 under stringent hybridization conditions.

In some embodiments, the APC-targeting ligand is ICAM-4 and comprises the amino acid sequence: ISRAGPGSLARGPLVRAFCHGVSVPSVAAVFFGGRLPGSWERAGTPDAGAKPQGPSR ALRDLSALLGAHEPGVRGCAAGEVSAAQLQQQLSPAAEFQPPHPAAARQDAQRAGLG VLPAARREGLELPRALPRDLRRKNTLGHLQDHRLQCSRWATWWPGMEAGSSIPKAW SASPAWIWPTPPTSLLLDPATSGSPSATRASISTAWWSATARHPLHCSLGAPRPQLWP PVPSLPLGSSSLWALRTYASALSPRRKGGCSMPAEREKEEYETIWGNGHTWWLTPVIP ALWEAEAGESLEPRSSRPAWTTDPVYAKNTQISLVWWPAPVVPATREAELGGSFEPK SRLQALIVPLHSSLGDRARPCLQKNKNKNKYWRGNPLESIKASLT (SEQ ID NO:5), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:5. Therefore, in some embodiments, the polynucleotide encoding the encoding the APC-targeting ligand has the nucleic acid sequence: ATCTCTAGAGCCGGCCCTGGCTCTCTGGCGCGGGGCCCCTTAGTCCGGGCTTTTT GCCATGGGGTCTCTGTTCCCTCTGTCGCTGCTGTTTTTTTTGGCGGCCGCCTACCC GGGAGTTGGGAGCGCGCTGGGACGCCGGACTAAGCGGGCGCAAAGCCCCAAGG GTAGCCCTCTCGCGCCCTCCGGGACCTCAGTGCCCTTCTGGGTGCGCATGAGCCC GGAGTTCGTGGCTGTGCAGCCGGGGAAGTCAGTGCAGCTCAATTGCAGCAACAGC TGTCCCCAGCCGCAGAATTCCAGCCTCCGCACCCCGCTGCGGCAAGGCAAGACGC TCAGAGGGCCGGGTTGGGTGTCTTACCAGCTGCTCGACGTGAGGGCCTGGAGCTC CCTCGCGCACTGCCTCGTGACCTGCGCAGGAAAAACACGCTGGGCCACCTCCAGG ATCACCGCCTACAGTGTTCCCGGTGGGCTACTTGGTGGTGACCCTGAGGCATGGA AGCCGGGTCATCTATTCCGAAAGCCTGGAGCGCTTCACCGGCCTGGATCTGGCCA ACGTGACCTTGACCTACGAGTTTGCTGCTGGACCCCGCGACTTCTGGCAGCCCGT GATCTGCCACGCGCGCCTCAATCTCGACGGCCTGGTGGTCCGCAACAGCTCGGCA CCCATTACACTGATGCTCGCTTGGAGCCCCGCGCCCACAGCTTTGGCCTCCGGTT CCATCGCTGCCCTTGTAGGGATCCTCCTCACTGTGGGCGCTGCGTACCTATGCAA GTGCCTAGCTATGAAGTCCCAGGCGTAAAGGGGGATGTTCTATGCCGGCTGAGCG AGAAAAAGAGGAATATGAAACAATCTGGGGAAATGGCCATACATGGTGGCTGACGC CTGTAATCCCAGCACTTTGGGAGGCCGAGGCAGGAGAATCGCTTGAGCCCAGGAG TTCGAGACCAGCCTGGACAACATAGTGAGACCCCGTCTATGCAAAAAATACACAAA TTAGCCTGGTGTGGTGGCCCGCACCTGTGGTCCCAGCTACCCGGGAGGCTGAGTT GGGAGGATCCTTTGAGCCCTGAAAGTCGAGGTTGCAGTGAGCCTTGATCGTGCCA CTGCACTCCAGCCTGGGGGACAGAGCACGACCCTGTCTCCAAAAATAAAATAAAAA TAAAAATAAATATTGGCGGGGGAACCCTCTGGAATCAATAAAGGCTTCCTTAACCA (SEQ ID NO:6), or a nucleic acid sequence that hybridizes to a nucleic acid sequence consisting of SEQ ID NO:6 under stringent hybridization conditions.

Also disclosed are polynucleotides comprising nucleic acid sequences encoding an APC-targeting ligand. In some embodiments, the APC-targeting ligand is FGF10 and comprises the amino acid sequence: GFPRDLEVERRAQQNASRQPPPKQEVAFGRLHINRHHQKEKGRRRQQRLGSCLQFQ LQRDTFVASRLGLCREGPEGANRRGAQMSSCTPTPPTLGFVHRPVICFSDLFCIHGEE RSEEENKVTPGGSEELWPTPPVPTAAATHVHCSPDWRDAGSGSEDGARTGSRSFLE VMDVGALASGQIRTQRELSRSHHLQLSLCLAAGLTLPVCSFDNFQCREFQYNVEMDTD TLCLSLSPPAGLLLLLLVALFGVFVPCHLPSSWSGHGVTGGHQLLFFLLVLLLSFQCGKA CAELQSPPRRCPLEKAVLLHQVLSHDEERQGQRDQERLSVQCPGDNISGNRSCCRQS HQQQLLLSHEQEGETLWLKRVQRLAERENRGKWIQHLCIFLAAQWQANVCGIEWKRS SQERTKNKKEKHLCSLPPHDDPNIEENTVGGCSTTNDSLDRKRWYPHRLLKRQRHSP EFSLFKGRKALDVFVLTADIQSSFHLCVIVSCLIIKIEASQVWMEVILKTMLWWGLGFFCL FVCFIFKFLFLNFDRTRTWNTLLNDLWESYLWNMNTYQRLSLLIQAFNEQDTQAFTGKH LGHIICTTKGALGVAPWKNWIRFTNINIVVNCPNTNSMVCLCILSPSFSISFVIYLIGCISFG VRVSSMLPSGLPFLSLSLSLSLSLSLSLSLSLSLSLSPSLSLPPPSGTIRTFMTKCFKTLA NTSEIGDEIKEAVMPHALSVDLICTSAIKNTSNDYGRILLIMQISVIICQHISVQRDPYLKMK AKFSFIVLFHLQEYTKRRKNELLDHCWVFFFLLMMACHRSQWQMMQRLSAHTALCKA HRILLPQKNQKRKFGMKCNSPWGLTANISYMSNHILTPVQAEWSSLYRTTTLFSVNLKC SRRFHNITTLKFIFLEKVKAVCKCAMHPRSVDHINTALFAVSVVILRMETLLKKFRITLRYC CSLLSCLEMSVYFHCLDSVFEQKDSPNVSVYNLTSREASDLSSSNLFVLMLDQYKSAF NIllKGSVILKLAFFFRMFLQVIIYDFFEKLAFQEKSTEAQCFGLWVVAFCGVLSTCFLTNG IFKMYTSCGWILYVRVLVTESKGSNVMNLKNVSFFTFILKLYLTHDHGPPTTKIKCLGRQ TIVFSDKGNSKCRRWILISLCHLDYRDYVTVIIIVLKTLNSFQNVSKLAESKNRKHLSPSPT LHFPLYASQRIQNEFGKERGDSSSLHICLILIENSGPFYNYKKRSSVLNFVCKKKRFCEF CLRGKCGHCSTMLKCVSHGYRAYDCHQTPRYRIASLLLILKRLNSVYYVTFKRQKCWE KRIKVLFLFYFLSFFSSLFFSFLFFFFSFFFLFFFYPKTIPGNEISSRAVCMAGCGMPHSP KQQQQKKKLLKHLGFFNVLHVAGYPYPNNNQMLSGSPNYSGPPSLHSGRRMNKQGW LVQQEALGGKPGAANVDFQGMEKTNEFDLKS (SEQ ID NO:7), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:7. Therefore, in some embodiments, the polynucleotide encoding the encoding the APC-targeting ligand has the nucleic acid sequence: GGCTTTCCAAGGGACTTGGAGGTGGAGAGAAGGGCCCAACAAAACGCCAGCCGC CAGCCGCCCCCCAAACAAGAAGTGGCTTTCGGAAGACTTCACATCAACAGGCACC ACCAAAAAGAGAAAGGAAGGAGAAGACAACAGCGCCTGGGCAGCTGCCTCCAGTT CTGACAACTCCAAAGAGACACTTTTTAAGTGGCCAGCAGGCTGGGACTCTGCAGAG AAGGACCAGAAGGTGCCAACCGCAGAGGGGCGCAGATGTCTTCCTGCACCCCCAC CCCACCCACTTTGGGTTTTGTTCACCGTCCTGTCATCTGTTTTTCAGACCTCTTTTG CATCTAACATGGTGAAGAAAGGAGTGAAGAAGAGAACAAAGTAACCCCCGGGGGG AGCGAAGAGCTCTGGTGACCGACACCACCAGTTCCTACTGCCGCGGCCACCCACG TCCACTGTTCACCCTGAGACTGGAGAGACGCAGGCAGCGGATCCGAGGACGGAG CGAGGACAGGCAGCCGGTCCTTCCTAGAAGTTATGGATGTTGGTGCACTCGCTTCT GGCCAGATCCGTACCCAGAGGGAGCTATCCAGAAGCCACCACCTCCAGCTGTCTC TCTGCCTCGCAGCAGGTCTTACCCTTCCAGTATGTTCCTTCTGATGAGACAATTTCC AGTGCCGAGAGTTTCAGTACAATGTGGAAATGGATACTGACACATTGTGCCTCAGC CTTTCCCCACCTGCCGGGCTGCTGTTGCTGCTTCTTGTTGCTCTTTTTGGTGTCTTC GTTCCCTGTCACCTGCCAAGCTCTTGGTCAGGACATGGTGTCACAGGAGGCCACC AACTGCTCTTCTTCCTCCTCGTCCTTCTCCTCTCCTTCCAGTGCGGGAAGGCATGT GCGGAGCTACAATCACCTCCAAGGAGATGTCCGCTGGAGAAGGCTGTTCTCCTTCA CCAAGTACTTTCTCACGATTGAGAAGAACGGCAAGGTCAGCGGGACCAAGAATGAA GACTGTCCGTACAGTGTCCTGGAGATAACATCAGTGGAAATCGGAGTTGTTGCCGT CAAAGCCATCAACAGCAACTATTACTTAGCCATGAACAAGAAGGGGAAACTCTATG GCTCAAAAGAGTTTAACAACGACTGTAAGCTGAAAGAGAGAATAGAGGAAAATGGA TACAACACCTATGCATCTTTTAACTGGCAGCACAATGGCAGGCAAATGTATGTGGC ATTGAATGGAAAAGGAGCTCCCAGGAGAGGACAAAAAACAAGAAGGAAAAACACCT CTGCTCACTTCCTCCCCATGACGATCCAAACATAGAAGAAAACACTGTTGGTGGAT GCAGTACAACCAATGACTCTTTGGACAGAAAGAGATGGTATCCTCACTGAAGACTG TAGCTCAAAAGGCAAAGACATAGCCCTGAATTCAGCTTGTTTAAAGGAAGGAAGGC TTTGGATGTTTTTGTACTCACTGCTGACATACAAAGTTCTTTTCACTAGCTCTGTGTC ATTGTGTCATGCCTTATAATCAAGATAGAGGCAAGTCAAGTTTGGATGGAAGTTATC CTCAAGTGAACAATGTTGTGGTGGGGGCTGGGCTTTTTTTGTTTGTTTGTTTGTTTC ATTTTTAAGTTTTTGTTTTTGAACTTCTGAGATAGAACTTAAAGAACATGGAACACTC TGTTGAATGATCTTTGGGAAAGTTATTTATGGAATATGAACACATATCAAAGACTTTC ATTGCTCATTCAAGCCTGATGATTCAATGAGCAGTAAGACACGCAAGCATTTACTGG AAAGCACTTGGGTCATATCATATGCACAACCAAAGGAGCTTTGGGTGTGGCACCAT GGAAGAATTGGATCAGATTTACAAATATAAACATAGTAGTATGAAACTGTCCTAATA CAAATAGTATGGTATGCTTGTGCATTCTGTCTCCATCCTTTTCTATTTCCTTCTAAGT TATTTATTTAATAGGATGTTAAATATCTTTTGGGGTTTAAAGAGTATCTTCAATGCTG CCCTCTGGTTTACCTTTTCTCTCTCTCTCTCTCTCTCTCTCTCTCTCTCTCTCTCTCT CTCTCTCTCTCTCTCTCTCTCTCTCTCTCTCTCCCTCTCTCTCCCTCCCTCCCCCCT CTGGCACCATACGCACATTCATGACAAAGTGTTTTAAAACCTTGGCAAACACTTCAG AAATAGGAGATGAGATCAAGGAAGCAGTATGAATGCCCCATGCGCTCTCAGTTGAC TTAATTTGCACTTCTGCAATAAAAAACACCAGCAATGACTATGGCAGAATTCTGCTA TAGATTATGTAACAGATATCTGTCATCATTTGTCAACATATATCAGTCCAGAGGGAC CCTTACCTTAAAATGTAGAAGGCCAAATTCTCTTTCATTGTCTTATTTCATCTTCAAG AATATACTAAAAGAAGAAAAAATGAATTGTTAGACTAACATTGTTGGGTTTTTTTTTT CCTACTGATGATGGCTTGCCACAGGTCACAATGGCAAATGATGCAAAGGTTATCTG CACATACATGAGCCCTTTGTAAGGCCCACAGAATCCTTCTCCCTCAAAAGAACCAAA AAAGGAAATTTGGTATGAAGTGCAACTCTCCCTGGGGCTTAACCTGAGCAAATATAT CCTAGTATATGAGTAACCATATACTGACACCTGTTCAAGCTGAATGGTCTAGTCTTT ACAGAACCACATAAACCTTGTTTTCTGTAAATTTAAAATGTTCTAGAAGGTTCCATAA TATAACCACATTGAAATTCATTTTCTTAGAAAAGGTATAGAAAGCAGTATGTAAGTGT GCCATGCACCCTCGCTCTGTAGATCACTAAATAAACACGTAAGCCTTATTTGCAGTG TCTGTAGTGATTTTAAGAATGTAGGAAACACTTCTAAAAAAATTTTAAAGGATAACTC TGAGATGATATTGATGCTGCAGTCTTCTTTCTTGTTTGGAAATGTCTGTTTATTTTCA TTGTTTGGATTCAGTATTTTGATAGGAACAAAAAGACTCACCAAATGTGTCTGTTTAC TAAAATTTAACCTCTAGAGAGGCTAGTGATTTGTGATCCTCTTCTAACTTATTTGTGC TGATGCTTGACCAGTACAAATCAGCTTTTTAAAATATTATTATTAAAGGTTGATCAGT CATTTTAAAATTGGCCTTTTTTTTCAGAATGTTCCTACAGGTCATAATTTATGATTTCT TTGAAAAGCTTGCATTTCAAGAGAAAAGCACAGAGGCACAATGCTTTGGTTTATGG GTATAGGTTGCATTTTGTGGTGTTCTTTCAACTTGTTTTCTGACAAATGGGATTTTTA AAATGTATACTTCTTGTGGTTGGATTCTGTATGTTAGAGTTTAATTGGTAACTGAGTC TAAAGGCTCTAATGTAATGAATCTCTAGAAGAACTAGGTATCTTTTTTTACTTTTATTT TAAAATAATAATTATACCTGACACATGACCATGGACCACCCACAACCAAAATTAAAT GTTTGGGGAGACAAACTATAGTATTCAGTGACAAGGGTAACAGCAAATAGTGCAGA CGTTGGATTCTTATTTCACTTTGCCATTTAGATTACTAAAGAGACTATGTGTAAACAG TCATCATTATAGTACTCAAGACATTAAACAGCTTCTAGCAAAATGTATCAAAGCTTGC AGAGTCCAAAAATAGAAAACATCTTTCCCCCTCTCCCACCCTACATTTCCCCCTGTA TGCATCCTAACAGAGATAAATACAAAATGAATTCGGTAAGGAGAGAGGAGATTCTTC TTCACTTCATATTTGTTTGATATTAATAGAGAATTCTGGTCCTTTTTACAACTACTGAA AGAAAAGAAGTTCAGTCCTAAATTTTGTGTGTTAAAAAAAGAAAAGATTTTGTGAGTT CTGCCTCCGTGGGAAGTGTGGGCACTGCTCCACCATGCTGAAGTGTGTTAGCCAC GGGTACAGAGCATATGACTGTTGACATCAGACTCCTTAAAGATACAGAATCGCTTC CCTCCTCCTAATCCTCAAAAGGCTGAACAGTGTATATTATGTTACATTTAAATAAAGG CAATAAAAATGCTGGGAAAAGAGAATAAAAGTACTGTTCTTATTTTATTTCCTTTCTT TCTTCTCTTCTCTTTTCTTTTCTTTCCTTTTCTTTTTTTTTTCCTTTTTTTTTCTTTTTTT TTTTTATTAGCCTAAAACTATACCTGGTAATGAGATCAGCTCCAGGGCTGTGTGCAT GGCAGGATGTGGTTAAATGCCCCACAGCCCCAAACAACAACAACAGAAAAAAAAAT TACTCAAACATTTGTAAGGTTTCTTTAATGTTTTACATGTGTGAGCCGGCTATCCTTA CCCTAATAACAACCAAATGCTTTCGGGTTCTCCTAACTACTCAGGTCCACCTAGTTT ACACAGTGGATAAAGAAGAATGAATTGAAAACAAGGATGGCTTGTGCAACAATGAG AGGCTCTTGGAGGAAAGCCAGGAGCTGCAAACGTTGACTTCCAGGGCATGGAAAA GACCAACGAATTTGATTTGAAAAGT (SEQ ID NO:8), or a nucleic acid sequence that hybridizes to a nucleic acid sequence consisting of SEQ ID NO:8 under stringent hybridization conditions.

Also disclosed are polynucleotides comprising nucleic acid sequences encoding an APC-targeting ligand. In some embodiments, the APC-targeting ligand is FGF7 and comprises the amino acid sequence: THTHKHTRAHTQRENPSACFMETIMILLENFSAEKFVATVERLKLHQADNRHGILIYPAV SNKTKVKQTASQQLNLLRTVFMRIYQQSYLRRNPVLLSGTKRIRLTIWKEQLLFLKSIYN SQIGRGQPRSNNQLKIHFHYVIHEHPEHYTIMHKWILTWILPTLLYRSCFHIICLVGTISLA CNDMTPEQMATNVNCSSPERHTRSYDYMEGGDIRVRRLFCRTQWYLRIDKRGKVKGT QEMKNNYNIMEIRTVAVGIVAIKGVESEFYLAMNKEGKLYAKKECNEDCNFKELILENHY NTYASAKWTHNGGEMFVALNQKGIPVRGKKTKKEQKTAHFLPMAITLHMVYKEPVPAG RFLVDCFLSSQNFLSFYFLVIKKGWKTTEKLIKLDLCIYVCFKTLHRKIKVYTKIRFSNRLKI VKLVVQSCQFFSINLPLRVCILSDNDYLNIPICLNGCYNNNNTDVVIGISDLQASGRICQII KPHLWKMSSILNAFKIIIYLNSNQKKNSQKNYYESQNRFNKSTGLEHAYTYKEQNFCCS SGKGIAKRMFPKILYIRQQLMIILYFILLATKHFINPQSKIEKSLSFFQVTSIFVFIFGNMAFN NVLPTNNHAFFLWLQHTLFVVFELYCFVIVYVIYKKKTLISCICFICFFRNNKTVWLNGKF PSLFLTLSLAHSTWASKSWKADKNKSLKQCLQMSHTEQYKYVKNLSPHILANLDHISKII TNISIYRILKLEYIMHGKISYFKTMYIIFSNLKSLLFKRWQHKLFSCMARAQCPKKMQLRIR GPECHEGLRSAYRQDYYKDEFPLQKSFIGRSWHFICSPMGGQYLSNLKGMLTTVVLIS KYLSFKSLYINSIWYIYRELYEKARSTKTIDSFNFPVVDLYDQDVENKELPFLRYTLGREH EWYSELSPDSRTLLARFGQASVTVVLAYGQRRTFFICVSLVPSRLSVHNQFSVIHMFID KRKYTLKLIFILKGNMTIQKFKKCIYINTSYVRTKCSLSMELYFHQITQCIRQNQHLVVNPF LVVVTKRLLEICTLIICFSPIFKFIMQILENKICSSYTPLEFNIKTVRGLHPSYSFVVEIElLIHL RSRSWVDIQLSQESRSVSTCFLLFDYYYLMVFGMFLFFISVEIPLHLYTNVTMSWQCTS YTMTNLYCDDLTQKEKRNYVVFNSDSYSPFVYEWKALCKIYIAEAFKCSLKDKIKYMSF QLEKKKLKHEMITKVNKRYFQSSEQNILTAIllllKIKTIVIVSLVGYKFNLMTEEKGCWRILR VSISSLLFWFSRLPKNIKMCEGTLKQSYNKKNSKTVPLTIFYNFSNSRGVKEVRECKGG PPQTEHFFSLKTHVIFASYSIIYLWLRYDNVCNTPVSKIDTSELAGLIMQFGLHPYSTAIYL SVNETQKIICLPSNFTHKHKAKENQNSNSPRIGVITKIFNSQYGLLANQNTSLIHRNGCR PLLTCGYHFYDHPKTELGKYLLTGLSGLNSQYVSYEIAHNFMCNYFSIVVSYSLFHFCTV FIQPSCCFQRSLVPSGTHILPPILFMVCQENLLSLELKNLTGSKKYSPSLFPITSDFSILKL HFSRNQIFTLIWEDASYVFLLLPGYHVVAFSKFLSLNLEPIKIYFLSFIFLKCEMKITFHLKT LLLMNPSMCLSIAVQIKTWLET (SEQ ID NO:9), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:9. Therefore, in some embodiments, the polynucleotide encoding the APC-targeting ligand has the nucleic acid sequence: ACACACACACACAAGCACACACGCGCTCACACACAGAGAGAAAATCCTTCTGCCTG TTGATTTATGGAAACAATTATGATTCTGCTGGAGAACTTTTCAGCTGAGAAATAGTTT GTAGCTACAGTAGAAAGGCTCAAGTTGCACCAGGCAGACAACAGACATGGAATTCT TATATATCCAGCTGTTAGCAACAAAACAAAAGTCAAATAGCAAACAGCGTCACAGCA ACTGAACTTACTACGAACTGTTTTTATGAGGATTTATCAACAGAGTTATTTAAGAGGA ATCCTGTGTTGTTATCAGGAACTAAAAGGATAAGGCTAACAATTTGGAAAGAGCAAC TACTCTTTCTTAAATCAATCTACAATTCACAGATAGGAAGAGGTCAATGACCTAGGA GTAACAATCAACTCAAGATTCATTTTCATTATGTTATTCATGAACACCCGGAGCACTA CACTATAATGCACAAATGGATACTGACATGGATCCTGCCAACTTTGCTCTACAGATC ATGCTTTCACATTATCTGTCTAGTGGGTACTATATCTTTAGCTTGCAATGACATGACT CCAGAGCAAATGGCTACAAATGTGAACTGTTCCAGCCCTGAGCGACACACAAGAAG TTATGATTACATGGAAGGAGGGGATATAAGAGTGAGAAGACTCTTCTGTCGAACAC AGTGGTACCTGAGGATCGATAAAAGAGGCAAAGTAAAAGGGACCCAAGAGATGAA GAATAATTACAATATCATGGAAATCAGGACAGTGGCAGTTGGAATTGTGGCAATCAA AGGGGTGGAAAGTGAATTCTATCTTGCAATGAACAAGGAAGGAAAACTCTATG CAA AGAAAGAATGCAATGAAGATTGTAACTTCAAAGAACTAATTCTGGAAAACCATTACA ACACATATGCATCAGCTAAATGGACACACAACGGAGGGGAAATGTTTGTTGCCTTA AATCAAAAGGGGATTCCTGTAAGAGGAAAAAAAACGAAGAAAGAACAAAAAACAGC CCACTTTCTTCCTATGGCAATAACTTAATTGCATATGGTATATAAAGAACCAGTTCCA GCAGGGAGATTTCTTTAAGTGGACTGTTTTCTTTCTTCTCAAAATTTTCTTTCCTTTT ATTTTTTAGTAATCAAGAAAGGCTGGAAAACTACTGAAAAACTGATCAAGCTGGACT TGTGCATTTATGTTTGTTTTAAGACACTGCATTAAAGAAAGATTTGAAAAGTATACAC AAAAATCAGATTTAGTAACTAAAGGTTGTAAAAAATTGTAAAACTGGTTGTACAATCA TGATGTTAGTAACAGTAATTTTTTTCTTAAATTAATTTACCCTTAAGAGTATGTTAGAT TTGATTATCTGATAATGATTATTTAAATATTCCTATCTGCTTATAAAATGGCTGCTATA ATAATAATAATACAGATGTTGTTATATAAGGTATATCAGACCTACAGGCTTCTGGCA GGATTTGTCAGATAATCAAGCCACACTAACTATGGAAAATGAGCAGCATTTTAAATG CTTTCTAGTGAAAAATTATAATCTACTTAAACTCTAATCAGAAAAAAAATTCTCAAAAA AACTATTATGAAAGTCAATAAAATAGATAATTTAACAAAAGTACAGGATTAGAACATG CTTATACCTATAAATAAGAACAAAATTTCTAATGCTGCTCAAGTGGAAAGGGTATTG CTAAAAGGATGTTTCCAAAAATCTTGTATATAAGATAGCAACAGTGATTGATGATAAT ACTGTACTTCATCTTACTTGCCACAAAATAACATTTTATAAATCCTCAAAGTAAAATT GAGAAATCTTTAAGTTTTTTTCAAGTAACATAATCTATCTTTGTATAATTCATATTTGG GAATATGGCTTTTAATAATGTTCTTCCCACAAATAATCATGCTTTTTTCCTATGGTTA CAGCATTAAACTCTATTTTAAGTTGTTTTTGAACTTTATTGTTTTGTTATTTAAGTTTAT GTTATTTATAAAAAAAAAACCTTAATAAGCTGTATCTGTTTCATATGCTTTTAATTTTA AAGGAATAACAAAACTGTCTGGCTCAACGGCAAGTTTCCCTCCCTTTTCTGACTGAC ACTAAGTCTAGCACACAGCACTTGGGCCAGCAAATCCTGGAAGGCAGACAAAAATA AGAGCCTGAAGCAATGCTTACAATAGATGTCTCACACAGAACAATACAAATATGTAA AAAATCTTTCACCACATATTCTTGCCAATTAATTGGATCATATAAGTAAAATCATTAC AAATATAAGTATTTACAGGATTTTAAAGTTAGAATATATTTGAATGCATGGGTAGAAA ATATCATATTTTAAAACTATGTATATTTAAATTTAGTAATTTTCTAATCTCTAGAAATCT CTGCTGTTCAAAAGGTGGCAGCACTGAAAGTTGTTTTCCTGTTAGATGGCAAGAGC ACAATGCCCAAAATAGAAGATGCAGTTAAGAATAAGGGGCCCTGAATGTCATGAAG GCTTGAGGTCAGCCTACAGATAACAGGATTATTACAAGGATGAATTTCCACTTCAAA AGTCTTTCATTGGCAGATCTTGGTAGCACTTTATATGTTCACCAATGGGAGGTCAAT ATTTATCTAATTTAAAAGGTATGCTAACCACTGTGGTTTTAATTTCAAAATATTTGTCA TTCAAGTCCCTTTACATAAATAGTATTTGGTAATACATTTATAGATGAGAGTTATATG AAAAGGCTAGGTCAACAAAAACAATAGATTCATTTAATTTTCCTGTGGTTGACCTATA CGACCAGGATGTAGAAAACTAGAAAGAACTGCCCTTCCTCAGATATACTCTTGGGA GAGAGCATGAATGGTATTCTGAACTATCACCTGATTCAAGGACTTTGCTAGCTAGGT TTTGAGGTCAGGCTTCAGTAACTGTAGTCTTGTGAGCATATTGAGGGCAGAGGAGG ACTTAGTTTTTCATATGTGTTTCCTTAGTGCCTAGCAGACTATCTGTTCATAATCAGT TTTCAGTGTGAATTCACTGAATGTTTATAGACAAAAGAAAATACACACTAAAACTAAT CTTCATTTTAAAAGGGTAAAACATGACTATACAGAAATTTAAATAGAAATAGTGTATA TACATATAAAATACAAGCTATGTTAGGACCAAATGCTCTTTGTCTATGGAGTTATACT TCCATCAAATTACATAGCAATGCTGAATTAGGCAAAACCAACATTTAGTGGTAAATC CATTCCTGGTAGTATAAGTCACCTAAAAAAGACTTCTAGAAATATGTACTTTAATTAT TTGTTTTTCTCCTATTTTTAAATTTATTATGCAAATTTTAGAAAATAAAATTTGCTCTAG TTACACACCTTTAGAATTCTAGAATATTAAAACTGTAAGGGGCCTCCATCCCTCTTA CTCATTTGTAGTCTAGGAAATTGAGATTTTGATACACCTAAGGTCACGCAGCTGGGT AGATATACAGCTGTCACAAGAGTCTAGATCAGTTAGCACATGCTTTCTACTCTTCGA TTATTAGTATTATTAGCTAATGGTCTTTGGCATGTTTTTGTTTTTTATTTCTGTTGAGA TATAGCCTTTACATTTGTACACAAATGTGACTATGTCTTGGCAATGCACTTCATACAC AATGACTAATCTATACTGTGATGATTTGACTCAAAAGGAGAAAAGAAATTATGTAGTT TTCAATTCTGATTCCTATTCACCTTTTGTTTATGAATGGAAAGCTTTGTGCAAAATAT ACATATAAGCAGAGTAAGCCTTTTAAAAATGTTCTTTGAAAGATAAAATTAAATACAT GAGTTTCTAACAATTAGAAAAGAAAAAATTAAAACATGAAATGATAACAAAAGTAAAC AAAAGATACTTTCAAAGCAGTGAACAAAACATTTTGACATAAGCCATAATATAAATTA TAATATAAAAAATAAAAACCATAGTATAAATTGTCAGCCTTTGAGTTGGCTACAAATT CAATTTAATGACAGAAGAGAAGGGATGCTGGAGGTAAATTCTTAGGGTTTCTATCTC ATAGAGTTTGCTCTTCTGGTTCTCTAGACTGCCAAAGAACATAAAGATGTGCGAGG GGACCTAGCTGTAGTAAAAGCAATCCTATAACAAGAAAAACTCTAAAACAGTGCCCC TTACGATTTTCTACTGAAATTTCTCTAATAGTAGAGGTGTAAAATAAGAAGTTAGAGA ATAATGCAAAGGGGGCCCACCACAGACGGAACATTTCTTTTCTCTTAAGACTCATGT GATTTTTGCATCTTACTCCATAATATATTTGTGGTTGCGTTAATATGACAATGTCTGC AATTAAACACCAGTAAGCAAAATTGATACATCAGAATGACTTGCAGGGCTTATCATG CAGTTTGGTTTACATCCCTACTCCACTGCCATTTACTTGAGCGTGAATGAGACACAA AAGATTATTTGCCTCCCATAATCCAACTTTACACATAAATAACACAAGGCTAAAGAAA ACCAGAACTCAAATTCACCACGCATAGGAGTGATAACAAAAATATTTAACAGTCAGT ATGGGTGATTACTGGCCAATCAGAATACATCACTGATACATCGAAATGGATGCAGG CCACTATGACTAACTTGTGGGTATCATTTCTATGATCACCCTAAAACAGAGTTGGGA AAATATCTATTAACTGGTCTCTCTGGTTTGAATTCTCAATATGTATCTTAATATGAAAT AGCTCATTAAAACTTCATGTGTAACTATTTCAGCATTGTTGTCAGCTACTCTTTATTC CACTTCTGTACAGTATTTATTCAACCAAGCTGCTGCTTTCAATGAAGGTCACTTGTT CCTTCAGGGACACATATACTCCCACCTATCCTTTAATTTTGAATGGTTTGTCAGGAA AATTTACTTTCTCTTGAGTTGAAAAACTTGACAGGAAGCAAGAAATAATACAGTCCTA GCCTCTTTCCAATAACATCTGATTTCTCCATTCTCAAACTACACTTCTCAAGGAACCA GATATTTACTCTCATCTGGGAAGATGCCTCTTATGTTTTCCTTTTACTTCCTGGTTAT CATGTGGTTGCATTTTCCAAGTTCTTATCATTGAATTTATGAGAGCCTATCAAAATTT ATTTTCTTTCATTTATATTCTAATAATTGAAATGTGAGATGAAAATAACATTTCACTTA TGAAAAACCCTTCTCTTGATGAATCCTTCCATGTGTTAGTTATCTATTGCTGTGTAAC AAATTAAAACTTAATGGCTTGAAACAAA (SEQ ID NO:10), or a nucleic acid sequence that hybridizes to a nucleic acid sequence consisting of SEQ ID NO:10 under stringent hybridization conditions.

Also disclosed are polynucleotides comprising nucleic acid sequences encoding an APC-targeting ligand. In some embodiments, the APC-targeting ligand is KRT14 and comprises the amino acid sequence: TRAPSLHSANCSLAHLPPLHHDHLQPPVHLLQLHEGLLRHRGRHRGRLQPHLLRPGRR VLPRPQHLRGRPVCLILPLLLWGSLRAGGRLWRWLQQQQQQLWWLWGRIWWWPWC WLGWWLWWWLCWWWASGGQEGDHAEPQPPGLLPGQGACSGGGQRRPGSEDPLV PEAAACDQRLQSLLQDHGPEEQDSHSHSGQCQCPSADQCPSGRGLPHQVDRVEPAH ECGSRHQWPAQGAGRTDPGQSPGDADEPEGGAGLPEEEPRGGDECPERPGGWRC QCGDGRCTWRGPEPHSERDAPVEDGREEPQGCRGMVLHQDRGAEPRGGHQQRAG AERQERDLGAPAHHAEPGDAAVPAQHESIPGEQPGGDQRSLLHAAGPDPGDDWQRG GAAGPAPLRDGAAEPGVQDPAGREDAAGAGDRHLPPPAGGRGRPPLLLPVLLWIAVI QRCDLLQPPNPHQGHGCARWQGGVHPRAGPSHQELRLPSPAQAEAPRVDTDPTGR SPLLPKHFTAGPCFTLTPSWQSIQLHYLSCI (SEQ ID NO:11), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:11. Therefore, in some embodiments, the polynucleotide encoding the APC-targeting ligand has the nucleic acid sequence:

(SEQ ID NO: 12)
ACCCGAGCACCTTCTCTTCACTCAGCCAACTGCTCGCTCGCTCACCTCC
CTCCTCTGCACCATGACCACCTGCAGCCGCCAGTTCACCTCCTCCAGCT
CCATGAAGGGCTCCTGCGGCATCGGGGGGGGCATCGGGGGGGGCTCCAG
CCGCATCTCCTCCGTCCTGGCCGGAGGGTCCTGCCGCGCCCCCAGCACC
TACGGGGGGGGCCTGTCTGTCTCATCCTCCCGCTTCTCCTCTGGGGGAG
CCTGCGGGCTGGGGGGCGGCTATGGCGGTGGCTTCAGCAGCAGCAGCAG
CAGCTTTGGTAGTGGCTTTGGGGGAGGATATGGTGGTGGCCTTGGTGCT
GGCTTGGGTGGTGGCTTTGGTGGTGGCTTTGCTGGTGGTGATGGGCTTC
TGGTGGGCAGTGAGAAGGTGACCATGCAGAACCTCAATGACCGCCTGGC
CTCCTACCTGGACAAGGTGCGTGCTCTGGAGGAGGCCAACGCCGACCTG
GAAGTGAAGATCCGTGACTGGTACCAGAGGCAGCGGCCTGCTGAGATCA
AAGACTACAGTCCCTACTTCAAGACCATTGAGGACCTGAGGAACAAGAT
TCTCACAGCCACAGTGGACAATGCCAATGTCCTTCTGCAGATTGACAAT
GCCCGTCTGGCCGCGGATGACTTCCGCACCAAGTATGAGACAGAGTTGA
ACCTGCGCATGAGTGTGGAAGCCGACATCAATGGCCTGCGCAGGGTGCT
GGACGAACTGACCCTGGCCAGAGCTGACCTGGAGATGCAGATTGAGAGC
CTGAAGGAGGAGCTGGCCTACCTGAAGAAGAACCACGAGGAGGAGATGA
ATGCCCTGAGAGGCCAGGTGGGTGGAGATGTCAATGTGGAGATGGACGC
TGCACCTGGCGTGGACCTGAGCCGCATTCTGAACGAGATGCGTGACCAG
TATGAGAAGATGGCAGAGAAGAACCGCAAGGATGCCGAGGAATGGTTCT
TCACCAAGACAGAGGAGCTGAACCGCGAGGTGGCCACCAACAGCGAGCT
GGTGCAGAGCGGCAAGAGCGAGATCTCGGAGCTCCGGCGCACCATGCAG
AACCTGGAGATTGAGCTGCAGTCCCAGCTCAGCATGAAAGCATCCCTGG
AGAACAGCCTGGAGGAGACCAAAGGTCGCTACTGCATGCAGCTGGCCCA
GATCCAGGAGATGATTGGCAGCGTGGAGGAGCAGCTGGCCCAGCTCCGC
TGCGAGATGGAGCAGCAGAACCAGGAGTACAAGATCCTGCTGGACGTGA
AGACGCGGCTGGAGCAGGAGATCGCCACCTACCGCCGCCTGCTGGAGGG
CGAGGACGCCCACCTCTCCTCCTCCCAGTTCTCCTCTGGATCGCAGTCA
TCCAGAGATGTGACCTCCTCCAGCCGCCAAATCCGCACCAAGGTCATGG
ATGTGCACGATGGCAAGGTGGTGTCCACCCACGAGCAGGTCCTTCGCAC
CAAGAACTGAGGCTGCCCAGCCCCGCTCAGGCCTAGGAGGCCCCCCGTG
TGGACACAGATCCCACTGGAAGATCCCCTCTCCTGCCCAAGCACTTCAC
AGCTGGACCCTGCTTCACCCTCACCCCCTCCTGGCAATCAATACAGCTT
CATTATCTGAGTTGCATAA,

or a nucleic acid sequence that hybridizes to a nucleic acid sequence consisting of SEQ ID NO:12 under stringent hybridization conditions.

Also disclosed are polynucleotides comprising nucleic acid sequences encoding an APC-targeting ligand. In some embodiments, the APC-targeting ligand is CD2 and comprises the amino acid sequence: SLTSVPFARAQNQKRKPTPKMSFPCKFVASFLLIFNVSSKGAVSKEITNALETWGALGQ DINLDIPSFQMSDDIDDIKWEKTSDKKKIAQFRKEKETFKEKDTYKLFKNGTLKIKHLKTD DQDIYKVSIYDTKGKNVLEKIFDLKIQERVSKPKISWTCINTTLTCEVMNGTDPELNLYQD GKHLKLSQRVITHKWTTSLSAKFKCTAGNKVSKESSVEPVSCPEKGLDIYLIIGICGGGS LLMVFVALLVFYITKRKKQRSRRNDEELETRAHRVATEERGRKPHQIPASTPQNPATSQ HPPPPPGHRSQAPSHRPPPPGHRVQHQPQKRPPAPSGTQVHQQKGPPLPRPRVQPK PPHGAAENSLSPSSNKRKLSFSIKSTVDFCPPDVHIRTSMRCFLCAEHCHLLRLWATAT SASSNSAMWSTSGVFGLLRELHHTSKEKQYKCDCKNGRGPSTEILEISCPLSGHVMRI KLVCLGLTTSSLSAETLEFLMCPGGHLPTILVKVKKLL (SEQ ID NO:13), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:13. Therefore, in some embodiments, the polynucleotide encoding the APC-targeting ligand has the nucleic acid sequence:

(SEQ ID NO: 14)
AGTCTCACTTCAGTTCCTTTTGCATGAAGAGCTCAGAATCAAAAGAGGA
AACCAACCCCTAAGATGAGCTTTCCATGTAAATTTGTAGCCAGCTTCCT
TCTGATTTTCAATGTTTCTTCCAAAGGTGCAGTCTCCAAAGAGATTACG
AATGCCTTGGAAACCTGGGGTGCCTTGGGTCAGGACATCAACTTGGACA
TTCCTAGTTTTCAAATGAGTGATGATATTGACGATATAAAATGGGAAAA
AACTTCAGACAAGAAAAAGATTGCACAATTCAGAAAAGAGAAAGAGACT
TTCAAGGAAAAAGATACATATAAGCTATTTAAAAATGGAACTCTGAAAA
TTAAGCATCTGAAGACCGATGATCAGGATATCTACAAGGTATCAATATA
TGATACAAAAGGAAAAAATGTGTTGGAAAAAATATTTGATTTGAAGATT
CAAGAGAGGGTCTCAAAACCAAAGATCTCCTGGACTTGTATCAACACAA
CCCTGACCTGTGAGGTAATGAATGGAACTGACCCCGAATTAAACCTGTA
TCAAGATGGGAAACATCTAAAACTTTCTCAGAGGGTCATCACACACAAG
TGGACCACCAGCCTGAGTGCAAAATTCAAGTGCACAGCAGGGAACAAAG
TCAGCAAGGAATCCAGTGTCGAGCCTGTCAGCTGTCCAGAGAAAGGTCT
GGACATCTATCTCATCATTGGCATATGTGGAGGAGGCAGCCTCTTGATG
GTCTTTGTGGCACTGCTCGTTTTCTATATCACCAAAAGGAAAAAACAGA
GGAGTCGGAGAAATGATGAGGAGCTGGAGACAAGAGCCCACAGAGTAGC
TACTGAAGAAAGGGGCCGGAAGCCCCACCAAATTCCAGCTTCAACCCCT
CAGAATCCAGCAACTTCCCAACATCCTCCTCCACCACCTGGTCATCGTT
CCCAGGCACCTAGTCATCGTCCCCCGCCTCCTGGACACCGTGTTCAGCA
CCAGCCTCAGAAGAGGCCTCCTGCTCCGTCGGGCACACAAGTTCACCAG
CAGAAAGGCCCGCCCCTCCCCAGACCTCGAGTTCAGCCAAAACCTCCCC
ATGGGGCAGCAGAAAACTCATTGTCCCCTTCCTCTAATTAAAAAAGATA
GAAACTGTCTTTTTCAATAAAAAGCACTGTGGATTTCTGCCCTCCTGAT
GTGCATATCCGTACTTCCATGAGGTGTTTTCTGTGTGCAGAACATTGTC
ACCTCCTGAGGCTGTGGGCCACAGCCACCTCTGCATCTTCGAACTCAGC
CATGTGGTCAACATCTGGAGTTTTTGGTCTCCTCAGAGAGCTCCATCAC
ACCAGTAAGGAGAAGCAATATAAGTGTGATTGCAAGAATGGTAGAGGAC
CGAGCACAGAAATCTTAGAGATTTCTTGTCCCCTCTCAGGTCATGTGTA
GATGCGATAAATCAAGTGATTGGTGTGCCTGGGTCTCACTACAAGCAGC
CTATCTGCTTAAGAGACTCTGGAGTTTCTTATGTGCCCTGGTGGACACT
TGCCCACCATCCTGTGAGTAAAAGTGAAATAAAAGCTTTGACTAGA,

or a nucleic acid sequence that hybridizes to a nucleic acid sequence consisting of SEQ ID NO:14 under stringent hybridization conditions.

Also disclosed are polynucleotides comprising nucleic acid sequences encoding an APC-targeting ligand. In some embodiments, the APC-targeting ligand is VLA-4 and comprises the amino acid sequence: VFEAPKNISCCWEYEDIDVECVLVCWRCINDSTCQTTRGSLFHDFRAVVSFFPLGREA NKLSHRLWRGTTLQYWLYICRSSLKDRYLSPGCELTQQSGRGPQYHSACYLKRGNGQ SKAQQSDCSNTFKIGADCSWVCKPNFICVWIKKANVHGGENELNFPCYQHWQYGSQC CGNNGTKFFPPNAVQHFGCPDYYWRMPLKLSKSVCIRAAKECNADLERHSPVLVQDE AIVLHKSSTLFKFLVFWENGKWKRSQCSYPTGRPAIHFRNGDFSTQVNKSNRFSRAKS KSNTKQGECCACSTGRTTSSKTQTLFHHSDYFKLATWTYCTSVDLICYVEGWLLKTIQI YPTRRKQKRQLELYQQKQLRTSFKLREWKTDSGCSKEIKTLFTRKNEFCLDFFYSSCDI LCLHARGKSQQLLFEIEELQRYSQRSLSFMGRETLKHSIYSRKVSPRYLEMKVLSIILEK STNTTYHMCLPQNEPHWVGRGSFQIHLYLFKICSLKIFFRELFPNFLTSGPLSLSPLFIIH FLRAVFQQPFFFSRLILYYRPNWQTSDTCTLVANDFWIIIFLLWISPSFFLYIYMCFYVGIY LPFFLSILPITHLYQAYPGVIFKSFVIFNKRLVLHFTYMLIKIQKPYLTNNFKIKAVQRLGGL YFICIMYYVKYLSHNYFLGCNSLLPFTSISVTLHGNEETKFINLNSILKDTDSICIQIMRRQ QNFHEMNIFIVCSYYMRFYFKLSGFKISLNTIIFVIFILCLSRLQNIILSDSVFIRELWPNFEH LLGVIKLEGNVEKQFWERFLYMKSLPLASHPNKLSVHRPAVMVRNVLRFAKAFENVST KPPEPRVCIHRNKLYDIYVFLKNFVSLKGSFFQENIDHRKIKPRFRTVFSRIVSKEIYIWFF PTQNYSETTIYFRLFEHSESRVLWLSNTGLSDTRGTQLHIGLDFAQFKIVFVINLLCYLYH EFNPTTLRRQGWVILFWQVGYITMFNLTALDGAVNQGRLMKSVLYLQNIGNLTLMSSN PEKKNPNYFRIIVKYCIMMVAKFFVCPINTLKKEFELIFKKKKK (SEQ ID NO:15), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:15. Therefore, in some embodiments, the polynucleotide encoding the APC-targeting ligand has the nucleic acid sequence: GTTTTTGAAGCCCCATGAAAATAAAACATATCTTGCTGTTGGGAGTATGAAGACATT GATGTTGAATGTGTCCTTGTTTAATGCTGGAGATGATGCATATGAAACGACTCTACA TGTCAAACTACCCGTGGGTCTTTATTTCATTAAGATTTTAGAGCTGTTGTTTCATTTT TCCCATTAGGAAGAGAAGCAAATAAACTGTGAAGTCACAGATAACTCTGGCGTGGT ACAACTTGACTGCAGTATTGGCTATATATATGTAGATCATCTCTCAAGGATAGATATT AGCTTTCTCCTGGATGTGAGCTCACTCAGCAGAGCGGAAGAGGACCTCAGTATCAC AGTGCATGCTACCTGTGAAAATGAAGAGGAAATGGACAATCTAAAGCACAGCAGAG TGACTGTAGCAATACCTTTAAAATATGAGGTTAAGCTGACTGTTCATGGGTTTGTAA ACCCAACTTCATTTGTGTATGGATCAAATGATGAAAATGAGCCTGAAACGTGCATGG TGGAGAAAATGAACTTAACTTTCCATGTTATCAACACTGGCAATAGTATGGCTCCCA ATGTTAGTGTGGAAATAATGGTACCAAATTCTTTTAGCCCCCAAACTGATAAGCTGT TCAACATTTTGGATGTCCAGACTACTACTGGAGAATGCCACTTTGAAAATTATCAAA GAGTGTGTGCATTAGAGCAGCAAAAGAGTGCAATGCAGACCTTGAAAGGCATAGTC CAGTTCTTGTCCAAGACTGATAAGAGGCTATTGTACTGCATAAAAGCTGATCCACAT TGTTTAAATTTCTTGTGTAATTTTGGGAAAATGGAAAGTGGAAAAGAAGCCAGTGTT CATATCCAACTGGAAGGCCGGCCATCCATTTTAGAAATGGATGAGACTTCAGCACT CAAGTTTGAAATAAGAGCAACAGGTTTTCCAGAGCCAAATCCAAGAGTAATTGAACT AAACAAGGATGAGAATGTTGCGCATGTTCTACTGGAAGGACTACATCATCAAAGAC CCAAACGTTATTTCACCATAGTGATTATTTCAAGTAGCTTGCTACTTGGACTTATTGT ACTTCTGTTGATCTCATATGTTATGTGGAAGGCTGGCTTCTTTAAAAGACAATACAA ATCTATCCTACAAGAAGAAAACAGAAGAGACAGTTGGAGTTATATCAACAGTAAAAG CAATGATGATTAAGGACTTCTTTCAAATTGAGAGAATGGAAAACAGACTCAGGTTGT AGTAAAGAAATTTAAAAGACACTGTTTACAAGAAAAAATGAATTTTGTTTGGACTTCT TTTACTCATGATCTTGTGACATATTATGTCTTCATGCAAGGGGAAAATCTCAGCAAT GATTACTCTTTGAGATAGAAGAACTGCAAAGGTAATAATACAGCCAAAGATAATCTC TCAGCTTTTAAATGGGTAGAGAAACACTAAAGCATTCAATTTATTCAAGAAAAGTAA GCCCTTGAAGATATCTTGAAATGAAAGTATAACTGAGTTAAATTATACTGGAGAAGT CTTAGACTTGAAATACTACTTACCATATGTGCTTGCCTCAGTAAAATGAACCCCACT GGGTGGGCAGAGGTTCATTTCAAATACATCTTTGATACTTGTTCAAAATATGTTCTTT AAAAATATAATTTTTTAGAGAGCTGTTCCCAAATTTTCTAACGAGTGGACCATTATCA CTTTAAAGCCCTTTATTTATAATACATTTCCTACGGGCTGTGTTCCAACAACCATTTT TTTTCAGCAGACTATGAATATTATAGTATTATAGGCCAAACTGGCAAACTTCAGACT GAACATGTACACTGGTTTGAGCTTAGTGAAATGACTTCTGGATAATTATTTTTTTATA ATTATGGATTTCACCATCTTTCTTTCTGTATATATACATGTGTTTTTATGTAGGTATAT ATTTACCATTCTTCCTATCTATTCTTCCTATAACACACCTTTATCAAGCATACCCAGG AGTAATCTTCAAATCTTTTGTTATATTCTGAAACAAAAGATTGTGAGTGTTGCACTTT ACCTGATACATGCTGATTTAGAAAATACAGAAACCATACCTCACTAATAACTTTAAAA TCAAAGCTGTGCAAAGACTAGGGGGCCTATACTTCATATGTATTATGTACTATGTAA AATATTGACTATCACACAACTATTTCCTTGGATGTAATTCTTTGTTACCCTTTACAAG TATAAGTGTTACCTTACATGGAAACGAAGAAACAAAATTCATAAATTTAAATTCATAA ATTTAGCTGAAAGATACTGATTCAATTTGTATACAGTGAATATAAATGAGACGACAG CAAAATTTTCATGAAATGTAAAATATTTTTATAGTTTGTTCATACTATATGAGGTTCTA TTTTAAATGACTTTCTGGATTTTAAAAAATTTCTTTAAATACAATCATTTTTGTAATATT TATTTTATGCTTATGATCTAGATAATTGCAGAATATCATTTTATCTGACTCTGTCTTCA TAAGAGAGCTGTGGCCGAATTTTGAACATCTGTTATAGGGAGTGATCAAATTAGAAG GCAATGTGGAAAAACAATTCTGGGAAAGATTTCTTTATATGAAGTCCCTGCCACTAG CCAGCCATCCTAATTGATGAAAGTTATCTGTTCACAGGCCTGCAGTGATGGTGAGG AATGTTCTGAGATTTGCGAAGGCATTTGAGTAGTGAAATGTAAGCACAAAACCTCCT GAACCCAGAGTGTGTATACACAGGAATAAACTTTATGACATTTATGTATTTTTAAAAA ACTTTGTATCGTTATAAAAAGGCTAGTCATTCTTTCAGGAGAACATCTAGGATCATA GATGAAAAATCAAGCCCCGATTTAGAACTGTCTTCTCCAGGATTGTCTCTAAGGAAA TTTACATTTGGTTCTTTCCTACTCAGAACTACTCAGAAACAACTATATATTTCAGGTT ATTTGAGCACAGTGAAAGCAGAGTACTATGGTTGTCCAACACAGGCCTCTCAGATA CAAGGGGAACACAATTACATATTGGGCTAGATTTTGCCCAGTTCAAAATAGTATTTG TTATCAACTTACTTTGTTACTTGTATCATGAATTTTAAAACCCTACCACTTTAAGAAGA CAGGGATGGGTTATTCTTTTTTGGCAGGTAGGCTATATAACTATGTGATTTTGAAAT TTAACTGCTCTGGATTAGGGAGCAGTGAATCAAGGCAGACTTATGAAATCTGTATTA TATTTGTAACAGAATATAGGAAATTTAACATAATTGATGAGCTCAAATCCTGAAAAAT GAAAGAATCCAAATTATTTCAGAATTATCTAGGTTAAATATTGATGTATTATGATGGT TGCAAAGTTTTTTGTGTGTCCAATAAACACATTGTAAAAAAAAGAATTTGAATTGATA TTTAAAAAAAAAAAAAAA (SEQ ID NO:16), or a nucleic acid sequence that hybridizes to a nucleic acid sequence consisting of SEQ ID NO:16 under stringent hybridization conditions.

Also disclosed are polynucleotides comprising nucleic acid sequences encoding an APC-targeting ligand. In some embodiments, the APC-targeting ligand is FLA-1 and comprises the amino acid sequence:

(SEQ ID NO: 17)
IIFLFHPVVASKSHGPPDAAPGATGPSSAGRMKDSCITVMAMALLSGFFFFAPASSYNL
DVRGARSFSPPRAGRHFGYRVLQVGNGVIVGAPGEGNSTGSLYQCQSGTGHCLPVTL
RGSNYTSKYLGMTLATDPTDGSILACDPGLSRTCDQNTYLSGLCYLFRQNLQGPMLQG
RPGFQECIKGNVDLVFLFDGSMSLQPDEFQKILDFMKDVMKKLSNTSYQFAAVQFSTS
YKTEFDFSDYVKRKDPDALLKHVKHMLLLTNTFGAINYVATEVFREELGARPDATKVLIII
TDGEATDSGNIDAAKDIIRYIIGIGKHFQTKESQETLHKFASKPASEFVKILDTFEKLKDLF
TELQKKIYVIEGTSKQDLTSFNMELSSSGISADLSRGHAVVGAVGAKDWAGGFLDLKAD
LQDDTFIGNEPLTPEVRAGYLGYTVTWLPSRQKTSLLASGAPRYQHMGRVLLFQEPQG
GGHWSQVQTIHGTQIGSYFGGELCGVDVDQDGETELLLIGAPLFYGEQRGGRVFIYQR
RQLGFEEVSELQGDPGYPLGRFGEAITALTDINGDGLVDVAVGAPLEEQGAVYIFNGRH
GGLSPQPSQRIEGTQVLSGIQWFGRSIHGVKDLEGDGLADVAVGAESQMIVLSSRPVV
DMVTLMSFSPAEIPVHEVECSYSTSNKMKEGVNITICFQIKSLIPQFQGRLVANLTYTLQL
DGHRTRRRGLFPGGRHELRRNIAVTTSMSCTDFSFHFPVCVQDLISPINVSLNFSLWEE
EGTPRDQRAQGKDIPPILRPSLHSETWEIPFEKNCGEDKKCEANLRVSFSPARSRALRL
TAFASLSVELSLSNLEEDAYWVQLDLHFPPGLSFRKVEMLKPHSQIPVSCEELPEESRL
LSRALSCNVSSPIFKAGHSVALQMMFNTLVNSSWGDSVELHANVTCNNEDSDLLEDNS
ATTIIPILYPINILIQDQEDSTLYVSFTPKGPKIHQVKHMYQVRIQPSIHDHNIPTLEAVVGV
PQPPSEGPITHQWSVQMEPPVPCHYEDLERLPDAAEPCLPGALFRCPVVFRQEILVQVI
GTLELVGEIEASSMFSLCSSLSISFNSSKHFHLYGSNASLAQVVMKVDVVYEKQMLYLY
VLSGIGGLLLLLLIFIVLYKVGFFKRNLKEKMEAGRGVPNGIPAEDSEQLASGQEAGDPG
CLKPLHEKDSESGGGKDVQACEVQSAQNWTQDAQGHSASACILPCALGRVTASPWP
SVSLSRTWNSFLPVSFAGSGRPAEGPAKRAAKVRACHYQTVHQPLLVSFLGRECLIM
WRNCSLRTGCSGPHPCPGMSTDASTPQNLSLHTPLHWSPVSSAGRKQMPVSLRDCG
TRLVLGQRPNSLACLPAPCKMRPSWPSPASSRAVMPPCSSGDTSLSPRPGSFLSSCIH
PDSSLCLNLPSRHPSFLDQQIPAHVTLGWVLTSFTLPPACTTPSKHTSCFFIRQPGCFF
PVLTYLAAISQTVRVKAILVLFTLGCLMICQGVGHLVGAHNFTELMTEPVGRYRKRGAG
LGAVVHACNPSTLGGQGGWITGQELEASLAKPHLYKYKIQAWWHTPVVPATQEVEVG
ELLEPGRWRLQAKIAPLHSSLGNTARLRLKEKIKIKSGHGPVTSPPLEAVFSGSALPLHT
LSQDPSRLCSGPHRKTELKVGTTSANLEPQCQAQCLHVFIQMNSV,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:17. Therefore, in some embodiments, the polynucleotide encoding the APC-targeting ligand has the nucleic acid sequence: ATCATTTTCCTCTTTCACCCTGTCTAGGTTGCCAGCAAATCCCACGGGCCTCCTGAC GCTGCCCCTGGGGCCACAGGTCCCTCGAGTGCTGGAAGGATGAAGGATTCCTGCA TCACTGTGATGGCCATGGCGCTGCTGTCTGGGTTCTTTTTCTTCGCGCCGGCCTCG AGCTACAACCTGGACGTGCGGGGCGCGCGGAGCTTCTCCCCACCGCGCGCCGGG AGGCACTTTGGATACCGCGTCCTGCAGGTCGGAAACGGGGTCATCGTGGGAGCTC CAGGGGAGGGGAACAGCACAGGAAGCCTCTATCAGTGCCAGTCGGGCACAGGAC ACTGCCTGCCAGTCACCCTGAGAGGTTCCAACTATACCTCCAAGTACTTGGGAATG ACCTTGGCAACAGACCCCACAGATGGAAGCATTTTGGCCTGTGACCCTGGGCTGT CTCGAACGTGTGACCAGAACACCTATCTGAGTGGCCTGTGTTACCTCTTCCGCCAG AATCTGCAGGGTCCCATGCTGCAGGGGCGCCCTGGTTTTCAGGAATGTATCAAGG GCAACGTAGACCTGGTATTTCTGTTTGATGGTTCGATGAGCTTGCAGCCAGATGAA TTTCAGAAAATTCTGGACTTCATGAAGGATGTGATGAAGAAACTCAGCAACACTTCG TACCAGTTTGCTGCTGTTCAGTTTTCCACAAGCTACAAAACAGAATTTGATTTCTCA GATTATGTTAAACGGAAGGACCCTGATGCTCTGCTGAAGCATGTAAAGCACATGTT GCTGTTGACCAATACCTTTGGTGCCATCAATTATGTCGCGACAGAGGTGTTCCGGG AGGAGCTGGGGGCCCGGCCAGATGCCACCAAAGTGCTTATCATCATCACGGATGG GGAGGCCACTGACAGTGGCAACATCGATGCGGCCAAAGACATCATCCGCTACATC ATCGGGATTGGAAAGCATTTTCAGACCAAGGAGAGTCAGGAGACCCTCCACAAATT TGCATCAAAACCCGCGAGCGAGTTTGTGAAAATTCTGGACACATTTGAGAAGCTGA AAGATCTATTCACTGAGCTGCAGAAGAAGATCTATGTCATTGAGGGCACAAGCAAA CAGGACCTGACTTCCTTCAACATGGAGCTGTCCTCCAGCGGCATCAGTGCTGACCT CAGCAGGGGCCATGCAGTCGTGGGGGCAGTAGGAGCCAAGGACTGGGCTGGGG GCTTTCTTGACCTGAAGGCAGACCTGCAGGATGACACATTTATTGGGAATGAACCA TTGACACCAGAAGTGAGAGCAGGCTATTTGGGTTACACCGTGACCTGGCTGCCCTC CCGGCAAAAGACTTCGTTGCTGGCCTCGGGAGCCCCTCGATACCAGCACATGGGC CGAGTGCTGCTGTTCCAAGAGCCACAGGGCGGAGGACACTGGAGCCAGGTCCAG ACAATCCATGGGACCCAGATTGGCTCTTATTTCGGTGGGGAGCTGTGTGGCGTCG ACGTGGACCAAGATGGGGAGACAGAGCTGCTGCTGATTGGTGCCCCACTGTTCTA TGGGGAGCAGAGAGGAGGCCGGGTGTTTATCTACCAGAGAAGACAGTTGGGGTTT GAAGAAGTCTCAGAGCTGCAGGGGGACCCCGGCTACCCACTCGGGCGGTTTGGA GAAGCCATCACTGCTCTGACAGACATCAACGGCGATGGGCTGGTAGACGTGGCTG TGGGGGCCCCTCTGGAGGAGCAGGGGGCTGTGTACATCTTCAATGGGAGGCACG GGGGGCTTAGTCCCCAGCCAAGTCAGCGGATAGAAGGGACCCAAGTGCTCTCAGG AATTCAGTGGTTTGGACGCTCCATCCATGGGGTGAAGGACCTTGAAGGGGATGGC TTGGCAGATGTGGCTGTGGGGGCTGAGAGCCAGATGATCGTGCTGAGCTCCCGGC CCGTGGTGGATATGGTCACCCTGATGTCCTTCTCTCCAGCTGAGATCCCAGTGCAT GAAGTGGAGTGCTCCTATTCAACCAGTAACAAGATGAAAGAAGGAGTTAATATCAC AATCTGTTTCCAGATCAAGTCTCTCATCCCCCAGTTCCAAGGCCGCCTGGTTGCCA ATCTCACTTACA CTCTGCAGCTGGATGGCCACCGGACCAGAAGACGGGGGTTG TT CCCAGGAGGGAGACATGAACTCAGAAGGAATATAGCTGTCACCACCAGCATGTCAT GCACTGACTTCTCATTTCATTTCCCGGTATGTGTTCAAGACCTCATCTCCCCCATCA ATGTTTCCCTGAATTTCTCTCTTTGGGAGGAGGAAGGGACACCGAGGGACCAAAGG GCGCAGGGCAAGGACATACCGCCCATCCTGAGACCCTCCCTGCACTCGGAAACCT GGGAGATCCCTTTTGAGAAGAACTGTGGGGAGGACAAGAAGTGTGAGGCAAACTT GAGAGTGTCCTTCTCTCCTGCAAGATCCAGAGCCCTGCGTCTAACTGCTTTTGCCA GCCTCTCTGTGGAGCTGAGCCTGAGTAACTTGGAAGAAGATGCTTACTGGGTCCAG CTGGACCTGCACTTCCCCCCGGGACTCTCCTTCCGCAAGGTGGAGATGCTGAAGC CCCATAGCCAGATACCTGTGAGCTGCGAGGAGCTTCCTGAAGAGTCCAGGCTTCT GTCCAGGGCATTATCTTGCAATGTGAGCTCTCCCATCTTCAAAGCAGGCCACTCGG TTGCTCTGCAGATGATGTTTAATACACTGGTAAACAGCTCCTGGGGGGACTCGGTT GAATTGCACGCCAATGTGACCTGTAACAATGAGGACTCAGACCTCCTGGAGGACAA CTCAGCCACTACCATCATCCCCATCCTGTACCCCATCAACATCCTCATCCAGGACC AAGAAGACTCCACACTCTATGTCAGTTTCACCCCCAAAGGCCCCAAGATCCACCAA GTCAAGCACATGTACCAGGTGAGGATCCAGCCTTCCATCCACGACCACAACATACC CACCCTGGAGGCTGTGGTTGGGGTGCCACAGCCTCCCAGCGAGGGGCCCATCAC ACACCAGTGGAGCGTGCAGATGGAGCCTCCCGTGCCCTGCCACTATGAGGATCTG GAGAGGCTCCCGGATGCAGCTGAGCCTTGTCTCCCCGGAGCCCTGTTCCGCTGCC CTGTTGTCTTCAGGCAGGAGATCCTCGTCCAAGTGATCGGGACTCTGGAGCTGGT GGGAGAGATCGAGGCCTCTTCCATGTTCAGCCTCTGCAGCTCCCTCTCCATCTCCT TCAACAGCAGCAAGCATTTCCACCTCTATGGCAGCAACGCCTCCCTGGCCCAGGTT GTCATGAAGGTTGACGTGGTGTATGAGAAGCAGATGCTCTACCTCTACGTGCTGAG CGGCATCGGGGGGCTGCTGCTGCTGCTGCTCATTTTCATAGTGCTGTACAAGGTTG GTTTCTTCAAACGGAACCTGAAGGAGAAGATGGAGGCTGGCAGAGGTGTCCCGAA TGGAATCCCTGCAGAAGACTCTGAGCAGCTGGCATCTGGGCAAGAGGCTGGGGAT CCCGGCTGCCTGAAGCCCCTCCATGAGAAGGACTCTGAGAGTGGTGGTGGCAAGG ACTGAGTCCAGGCCTGTGAGGTGCAGAGTGCCCAGAACTGGACTCAGGATGCCCA GGGCCACTCTGCCTCTGCCTGCATTCTGCCGTGTGCCCTCGGGCGAGTCACTGCC TCTCCCTGGCCCTCAGTTTCCCTATCTCGAACATGGAACTCATTCCTGCCTGTCTCC TTTGCAGGCTCATAGGGAAGACCTGCTGAGGGACCAGCCAAGAGGGCTGCAAAAG TGAGGGCTTGTCATTACCAGACGGTTCACCAGCCTCTCTTGGTTTCCTTCCTTGGA AGAGAATGTCTGATCTAAATGTGGAGAAACTGTAGTCTCAGGACCTAGGGATGTTC TGGCCCTCACCCCTGCCCTGGGATGTCCACAGATGCCTCCACCCCCCAGAACCTG TCCTTGCACACTCCCCTGCACTGGAGTCCAGTCTCTTCTGCTGGCAGAAAGCAAAT GTGACCTGTGTCACTACGTGACTGTGGCACACGCCTTGTTCTTGGCCAAAGACCAA ATTCCTTGGCATGCCTTCCAGCACCCTGCAAAATGAGACCCTCGTGGCCTTCCCCA GCCTCTTCTAGAGCCGTGATGCCTCCCTGTTGAAGCTCTGGTGACACCAGCCTTTC TCCCAGGCCAGGCTCCTTCCTGTCTTCCTGCATTCACCCAGACAGCTCCCTCTGCC TGAACCTTCCATCTCGCCACCCCTCCTTCCTTGACCAGCAGATCCCAGCTCACGTC ACACTTGGTTGGGTCCTCACATCTTTCACACTTCCACCAGCCTGCACTACTCCCTCA AAGCACACGTCATGTTTCTTCATCCGGCAGCCTGGATGTTTTTTCCCTGTTTAATGA TTGACGTACTTAGCAGCTATCTCTCAGTGAACTGTGAGGGTAAAGGCTATACTTGTC TTGTTCACCTTGGGATGATGCCTCATGATATGTCAGGGCGTGGGACATCTAGTAGG TGCTTGACATAATTTCACTGAATTAATGACAGAGCCAGTGGGAAGATACAGAAAAAG AGGGGCTGGGCTGGGCGCGGTGGTTCACGCCTGTAATCCCAGCACTTTGGGAGG CCAAGGAGGGTGGATCACCTGAGGTCAGGAGTTAGAGGCCAGCCTGGCGAAACC CCATCTCTACTAAAAATACAAAATCCAGGCGTGGTGGCACACACCTGTAGTCCCAG CTACTCAGGAGGTTGAGGTAGGAGAATTGCTTGAACCTGGGAGGTGGAGGTTGCA GTGAGCCAAGATTGCGCCATTGCACTCCAGCCTGGGCAACACAGCGAGACTCCGT CTCAAGGAAAAAATAAAAATAAAAAGCGGGCACGGGCCCGTGACATCCCCACCCTT GGAGGCTGTCTTCTCAGGCTCTGCCCTGCCCTAGCTCCACACCCTCTCCCAGGAC CCATCACGCCTGTGCAGTGGCCCCCACAGAAAGACTGAGCTCAAGGTGGGAACCA CGTCTGCTAACTTGGAGCCCCAGTGCCAAGCACAGTGCCTGCATGTATTTATCCAA TAAATGTGAAATTCTGTCCA (SEQ ID NO:18), or a nucleic acid sequence that hybridizes to a nucleic acid sequence consisting of SEQ ID NO:18 under stringent hybridization conditions.

Also disclosed are polynucleotides comprising nucleic acid sequences encoding an APC-targeting ligand. In some embodiments, the APC-targeting ligand is CD154 and comprises the amino acid sequence: NPEGGHFDSLLMLPLPPSLPEDTISTLTQHDRNIQPNFSPICGHWTAHQHENFYVFTYC FSYHPDDWVSTFCCVSSKVGQDRRKESSRFCIHENDTEMQHRRKILILTELGDKPVRL CEGYNVKQRGDEERKQLNAKRSESSNCGTCHKGQQNNICVTVGKRILHHEQQLGNPG KWETADRKTRTLLYLCPSHLLFQSGSFESSSIYSQPLPKVPRIRENLTQSCKYPQFRQT LRATIHSLGRSIIATRCFGVCQCDSKPSEPWHWLHVLWLTQTLNSVTLQAVVELTLGVFI IQHSGAHPLLTAYLPDPPYGELFI IHSKACRTVISELQVTNQNGPCSIRAYISEAATPLMQ TSRESYEKTRPLCTGILSKQQITCQVQFCFFACSVFPWIMHLIYQRCRREMGSLSSHSV MVDSGFLWPCWRGPGSRTSNTVENRNPPPPPATLSDSYSFSFNLSLSISLFQSLSLNL FLPISLSQSLCFPLSVSSLPQSLFSIPLSNTHTHTHTHTHTHTHTHTESGRCSVLFFPPC PYLYHYRGGVGSAALSLPTPHYEMTVFKGNLLYLPAVSIVSRVNLLSCYLFFERPLNI (SEQ ID NO:19), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:19. Therefore, in some embodiments the polynucleotide encoding the APC-targeting ligand has the nucleic acid sequence: AATCCTGAGTAAGGTGGCCACTTTGACAGTCTTCTCATGCTGCCTCTGCCACCTTCT CTGCCAGAAGATACCATTTCAACTTTAACACAGCATGATCGAAACATACAACCAAAC TTCTCCCCGATCTGCGGCCACTGGACTGCCCATCAGCATGAAAATTTTTATGTATTT ACTTACTGTTTTTCTTATCACCCAGATGATTGGGTCAGCACTTTTTGCTGTGTATCTT CATAGAAGGTTGGACAAGATAGAAGATGAAAGGAATCTTCATGAAGATTTTGTATTC ATGAAAACGATACAGAGATGCAACACAGGAGAAAGATCCTTATCCTTACTGAACTGT GAGGAGATTAAAAGCCAGTTTGAAGGCTTTGTGAAGGATATAATGTTAAACAAAGAG GAGACGAAGAAAGAAAACAGCTTTGAAATGCAAAAAGGTGATCAGAATCCTCAAATT GCGGCACATGTCATAAGTGAGGCCAGCAGTAAAACAACATCTGTGTTACAGTGGGC TGAAAAAGGATACTACACCATGAGCAACAACTTGGTAACCCTGGAAAATGGGAAAC AGCTGACCGTTAAAAGACAAGGACTCTATTATATCTATGCCCAAGTCACCTTCTGTT CCAATCGGGAAGCTTCGAGTCAAGCTCCATTTATAGCCAGCCTCTGCCTAAAGTCC CCCGGTAGATTCGAGAGAATCTTACTCAGAGCTGCAAATACCCACAGTTCCGCCAA ACCTTGCGGGCAACAATCCATTCACTTGGGAGGAGTATTTGAATTGCAACCAGGTG CTTCGGTGTTTGTCAATGTGACTGATCCAAGCCAAGTGAGCCATGGCACTGGCTTC ACGTCCTTTGGCTTACTCAAACTCTGAACAGTGTCACCTTGCAGGCTGTGGTGGAG CTGACGCTGGGAGTCTTCATAATACAGCACAGCGGTTAAGCCCACCCCCTGTTAAC TGCCTATTTATAACCCTAGGATCCTCCTTATGGAGAACTATTTATTATACACTCCAAG GCATGTAGAACTGTAATAAGTGAATTACAGGTCACATGAAACCAAAACGGGCCCTG CTCCATAAGAGCTTATATATCTGAAGCAGCAACCCCACTGATGCAGACATCCAGAG AGTCCTATGAAAAGACAAGGCCATTATGCACAGGTTGAATTCTGAGTAAACAGCAG ATAACTTGCCAAGTTCAGTTTTGTTTCTTTGCGTGCAGTGTCTTTCCATGGATAATG CATTTGATTTATCAGTGAAGATGCAGAAGGGAAATGGGGAGCCTCAGCTCACATTC AGTTATGGTTGACTCTGGGTTCCTATGGCCTTGTTGGAGGGGGCCAGGCTCTAGAA CGTCTAACACAGTGGAGAACCGAAACCCCCCCCCCCCCCCCGCCACCCTCTCGGA CAGTTATTCATTCTCTTTCAATCTCTCTCTCTCCATCTCTCTCTTTCAGTCTCTCTCTC TCAACCTCTTTCTTCCAATCTCTCTTTCTCAATCTCTCTGTTTCCCTTTGTCAGTCTCT TCCCTCCCCCAGTCTCTCTTCTCAATCCCCCTTTCTAACACACACACACACACACAC ACACACACACACACACACACACACACACACACAGAGTCAGGCCGTTGCTAGTCAGT TCTCTTCTTTCCACCCTGTCCCTATCTCTACCACTATAGATGAGGGTGAGGAGTAGG GAGTGCAGCCCTGAGCCTGCCCACTCCTCATTACGAAATGACTGTATTTAAAGGAA ATCTATTGTATCTACCTGCAGTCTCCATTGTTTCCAGAGTGAACTTGTAATTATCTTG TTATTTATTTTTTGAATAATAAAGACCTCTTAACATTA (SEQ ID NO:20), or a nucleic acid sequence that hybridizes to a nucleic acid sequence consisting of SEQ ID NO:20 under stringent hybridization conditions.

Also disclosed are polynucleotides comprising nucleic acid sequences encoding an APC-targeting ligand. In some embodiments, the APC-targeting ligand is CXC5 and comprises the amino acid sequence: TVLRILQSSLLQSPLLHPVQEPATARSALLTTMSLLSSRAARVPGPSSSLCALLVLLLLLT QPGPIASAGPAAAVLRELRCVCLQTTQGVHPKMISNLQVFAIGPQCSKVEVVASLKNGK EICLDPEAPFLKKVIQKILDGGNKENLREMSTHGKVSQSSAEKFSGGLTQGRQEGKILLL FVYLFFQLAFFLDSSLRVGKPMFAAAFSSANEVFSIVPLLFAVILSAMLLKFWQLTIVARN HWLLIFQSVLNCRLLYFQEIFLKILTEKAVDLMWKCFIRILLMElHCYLHFYKKEIFCFLGN MLENFLTLDCGILFNYFTLESVFHTLSMNIFPYSEFLKVVLGLISYLPIlLDILYLFSMANCH HLLLNFDFICYLLSILLGVPFWLNIYFRMKKLENRQIPDCFIKCILLVFKVKANLTMTCTLK VLETYSNNLNINLSFSYKNIRHPRGPSISPWIGDQRELGMLKTKQNKKKQGEVVQGMSI FYPSVWVRFSKIIIRRPAFMVEYIIIYKVATLGQVPSPLTALAPFTENLGRIAEDERERGG QGRCLSGFHSSFHWDFEAFLSECKACSSPGGTHWGWGWGKMRNRLVSVVLISLIMLS LFLQIFLFKLFHLCLEILPFRENVTLVKRLVGKLLPFFLTFKQTVINGCEFLFLLCFTFVFQ NRILYLNGKNKTFCAINKATARKIKHFLVKTYVFIYYIFIYNIYYIFSIAELFRCLLCIFRFPFC YELHIYYIHYIKIVLFYYVSHWFIVFILSFETLKDFTS (SEQ ID NO:21), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:21. Therefore, in some embodiments, the polynucleotide encoding the APC-targeting ligand has the nucleic acid sequence: ACAGTGCTCCGGATCCTCCAATCTTCGCTCCTCCAATCTCCGCTCCTCCACCCAGT TCAGGAACCCGCGACCGCTCGCAGCGCTCTCTTGACCACTATGAGCCTCCTGTCC AGCCGCGCGGCCCGTGTCCCCGGTCCTTCGAGCTCCTTGTGCGCGCTGTTGGTGC TGCTGCTGCTGCTGACGCAGCCAGGGCCCATCGCCAGCGCTGGTCCTGCCGCTG CTGTGTTGAGAGAGCTGCGTTGCGTTTGTTTACAGACCACGCAAGGAGTTCATCCC AAAATGATCAGTAATCTGCAAGTGTTCGCCATAGGCCCACAGTGCTCCAAGGTGGA AGTGGTAGCCTCCCTGAAGAACGGGAAGGAAATTTGTCTTGATCCAGAAGCCCCTT TTCTAAAGAAAGTCATCCAGAAAATTTTGGACGGTGGAAACAAGGAAAACTGATTAA GAGAAATGAGCACGCATGGAAAAGTTTCCCAGTCTTCAGCAGAGAAGTTTTCTGGA GGTCTCTGAACCCAGGGAAGACAAGAAGGAAAGATTTTGTTGTTGTTTGTTTATTTG TTTTTCCAGTAGTTAGCTTTCTTCCTGGATTCCTCACTTTGAAGAGTGTGAGGAAAA CCTATGTTTGCCGCTTAAGCTTTCAGCTCAGCTAATGAAGTGTTTAGCATAGTACCT CTGCTATTTGCTGTTATTTTATCTGCTATGCTATTGAAGTTTTGGCAATTGACTATAG TGTGAGCCAGGAATCACTGGCTGTTAATCTTTCAAAGTGTCTTGAATTGTAGGTGAC TATTATATTTCCAAGAAATATTCCTTAAGATATTAACTGAGAAGGCTGTGGATTTAAT GTGGAAATGATGTTTCATAAGAATTCTGTTGATGGAAATACACTGTTATCTTCACTTT TATAAGAAATAGGAAATATTTTAATGTTTCTTGGGGAATATGTTAGAGAATTTCCTTA CTCTTGATTGTGGGATACTATTTAATTATTTCACTTTAGAAAGCTGAGTGTTTCACAC CTTATCTATGTAGAATATATTTCCTTATTCAGAATTTCTAAAAGTTTAAGTTCTATGAG GGCTAATATCTTATCTTCCTATAATTTTAGACATTCTTTATCTTTTTAGTATGGCAAAC TGCCATCATTTACTTTTAAACTTTGATTTTATATGCTATTTATTAAGTATTTTATTAGG AGTACCATAATTCTGGTAGCTAAATATATATTTTAGATAGATGAAGAAGCTAGAAAAC AGGCAAATTCCTGACTGCTAGTTTATATAGAAATGTATTCTTTTAGTTTTTAAAGTAA AGGCAAACTTAACAATGACTTGTACTCTGAAAGTTTTGGAAACGTATTCAAACAATTT GAATATAAATTTATCATTTAGTTATAAAAATATATAGCGACATCCTCGAGGCCCTAGC ATTTCTCCTTGGATAGGGGACCAGAGAGAGCTTGGAATGTTAAAAACAAAACAAAA CAAAAAAAAACAAGGAGAAGTTGTCCAAGGGATGTCAATTTTTTATCCCTCTGTATG GGTTAGATTTTCCAAAATCATAATTTGAAGAAGGCCAGCATTTATGGTAGAATATATA ATTATATATAAGGTGGCCACGCTGGGGCAAGTTCCCTCCCCACTCACAGCTTTGGC CCCTTTCACAGAGTAGAACCTGGGTTAGAGGATTGCAGAAGACGAGCGGGAGCGG GGAGGGCAGGGAAGATGCCTGTCGGGTTTTTAGCACAGTTCATTTCACTGGGATTT TGAAGCATTTCTGTCTGAATGTAAAGCCTGTTCTAGTCCTGGTGGGACACACTGGG GTTGGGGGTGGGGGAAGATGCGGTAATGAAACCGGT TAGTCAGTGTTGTCTTAATA TCCTTGATAATGCTGTAAAGTTTATTTTTACAAATATTTCTGTTTAAGCTATTTCACCT TTGTTTGGAAATCCTTCCCTTTTAAAGAGAAAATGTGACACTTGTGAAAAGGCTTGT AGGAAAGCTCCTCCCTTTTTTTCTTTAAACCTTTAAATGACAAACCTAGGTAATTAAT GGTTGTGAATTTCTATTTTTGCTTTGTTTTTAATGAACATTTGTCTTTCAGAATAGGAT TCTGTGATAATATTTAAATGGCAAAAACAAAACATAATTTTGTGCAATTAACAAAGCT ACTGCAAGAAAAATAAAACATTTCTTGGTAAAAACGTATGTATTTATATATTATATATT TATATATAATATATATTATATATTTAGCATTGCTGAGCTTTTTAGATGCCTATTGTGTA TCTTTTAAAGGTTTTGACCATTTTGTTATGAGTAATTACATATATATTACATTCACTAT ATTAAAATTGTACTTTTTTACTATGTGTCTCATTGGTTCATAGTCTTTATTTTGTCCTT TGAATAAACATTAAAAGATTTCTAAACTTCA (SEQ ID NO:22), or a nucleic acid sequence that hybridizes to a nucleic acid sequence consisting of SEQ ID NO:22 under stringent hybridization conditions.

Also disclosed are polynucleotides comprising nucleic acid sequences encoding an APC-targeting ligand. In some embodiments, the APC-targeting ligand is CD22 and comprises the amino acid sequence: TRKQACTQTRHHASPRPLAPAPGSRILGFLLKMGFAPNPLRLGGGLRLDPLHLQSPRW PGKLHPVPQSVQEHLEVWDKTLKHKGWEGSFAEKGAIPGRQEELHTEYPPGAPQQW SAGAEDGVQDEMDGTNTPQCLKAFSTSYPAPSRNSRVPGSHSDLLAEFLLLWVSDPIA VAPRGGSNEAGCCHLDLLDHQVCLHPERAQVLPTVESPWEDCDLPASGCRWEVPLQ HGAAEREASSQEGDHSDSKPHADSRRRHSDPFLLQFQPQCYPVMETPWRLGGAIAW GAEDPKRWLGQHNHRLRSLLVLVGLPCRPECPVCPPRREGPENQAPFRDSLWKLGQ PPMLLKQPPQRSPVLLGEKWQASGERKPAEFLHLPRRCWELQLLGEQLHRTDSVQGL DTSAVCTQEAACVHEPGGPSDGGEECNPDLERRQPSRLPLHLVLEPKPPLPQPEAEIG AGEGPALGCLLVPGDQQCGQGPFASQHPHRLLPGDHRQASGCGTRVLPRHPHPGNL WAQAPATLEEDTEPAGASGEFQRPELLCEEKGKGPPLRPPLPGMLQSNDGRWHLHH PALSRDEHTTNWRCRVLRDAETSPGLRHGHLFSIAQAPSGHYENVIPDFPEDEGIHYSE LIQFGVGERPQAQENVDYVILKHHWMGCSRGTGGSGGQGSPRVFPR (SEQ ID NO:23), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:23. Therefore, in some embodiments, the polynucleotide encoding the APC-targeting ligand has the nucleic acid sequence: ACGCGGAAACAGGCTTGCACCCAGACACGACACCATGCATCTCCTCGGCCCCTGG CTCCTGCTCCTGGTTCTAGAATACTTGGCTTTCTCTGACTCAAGTAAATGGGTTTTT GAGCACCCTGAAACCCTCTACGCCTGGGAGGGGGCCTGCGTCTGGATCCCCTGCA CCTACAGAGCCCTAGATGGTGACCTGGAAAGCTTCATCCTGTTCCACAATCCTGAG TATAACAAGAACACCTCGAAGTTTGATGGGACAAGACTCTATGAAAGCACAAAGGA TGGGAAGGTTCCTTCTGAGCAGAAAAGGGTGCAATTCCTGGGAGACAAGAATAAGA ACTGCACACTGAGTATCCACCCGGTGCACCTCAATGACAGTGGTCAGCTGGGGCT GAGGATGGAGTCCAAGACTGAGAAATGGATGGAACGAATACACCTCAATGTCTCTG AAAGGCCTTTTCCACCTCATATCCAGCTCCCTCCAGAAATTCAAGAGTCCCAGGAA GTCACTCTGACCTGCTTGCTGAATTTCTCCTGCTATGGGTATCCGATCCAATTGCAG TGGCTCCTAGAGGGGGTTCCAATGAGGCAGGCTGCTGTCACCTCGACCTCCTTGA CCATCAAGTCTGTCTTCACCCGGAGCGAGCTCAAGTTCTCCCCACAGTGGAGTCAC CATGGGAAGATTGTGACCTGCCAGCTTCAGGATGCAGATGGGAAGTTCCTCTCCAA TGACACGGTGCAGCTGAACGTGAAGCATCCTCCCAAGAAGGTGACCACAGTGATT CAAAACCCCATGCCGATTCGAGAAGGAGACACAGTGACCCTTTCCTGTAACTACAA TTCCAGTAACCCCAGTGTTACCCGGTATGAATGGAAACCCCATGGCGCCTGGGAG GAGCCATCGCTTGGGGTGCTGAAGATCCAAAACGTTGGCTGGGACAACACAACCA TCGCCTGCGCAGCTTGTAATAGTTGGTGCTCGTGGGCCTCCCCTGTCGCCCTGAAT GTCCAGTATGCCCCCCGAGACGTGAGGGTCCGGAAAATCAAGCCCCTTTCCGAGA TTCACTCTGGAAACTCGGTCAGCCTCCAATGTGACTTCTCAAGCAGCCACCCCAAA GAAGTCCAGTTCTTCTGGGAGAAAAATGGCAGGCTTCTGGGGAAAGAAAGCCAGC TGAATTTTGACTCCATCTCCCCAGAAGATGCTGGGAGTTACAGCTGCTGGGTGAAC AACTCCATAGGACAGACAGCGTCCAAGGCCTGGACACTTGAAGTGCTGTATGCACC CAGGAGGCTGCGTGTGTCCATGAGCCCGGGGGACCAAGTGATGGAGGGGAAGAG TGCAACCCTGACCTGTGAGAGCGACGCCAACCCTCCCGTCTCCCACTACACCTGG TTTGACTGGAATAACCAAAGCCTCCCCTACCACAGCCAGAAGCTGAGATTGGAGCC GGTGAAGGTCCAGCACTCGGGTGCCTACTGGTGCCAGGGGACCAACAGTGTGGG CAAGGGCCGTTCGCCTCTCAGCACCCTCACCGTCTACTATAGCCCGGAGACCATC GGCAGGCGAGTGGCTGTGGGACTCGGGTCCTGCCTCGCCATCCTCATCCTGGCAA TCTGTGGGCTCAAGCTCCAGCGACGTTGGAAGAGGACACAGAGCCAGCAGGGGCT TCAGGAGAATTCCAGCGGCCAGAGCTTCTTTGTGAGGAATAAAAAGGTTAGAAGGG CCCCCCTCTCTGAAGGCCCCCACTCCCTGGGATGCTACAATCCAATGATGGAAGAT GGCATTAGCTACACCACCCTGCGCTTTCCCGAGATGAACATACCACGAACTGGAGA TGCAGAGTCCTCAGAGATGCAGAGACCTCCCCCGGACTGCGATGACACGGTCACT TATTCAGCATTGCACAAGCGCCAAGTGGGCACTATGAGAACGTCATTCCAGATTTT CCAGAAGATGAGGGGATTCATTACTCAGAGCTGATCCAGTTTGGGGTCGGGGAGC GGCCTCAGGCACAAGAAAATGTGGACTATGTGATCCTCAAACATTGACACTGGATG GGCTGCAGCAGAGGCACTGGGGGCAGCGGGGGCCAGGGAAGTCCCCGAGTTTTC CCCAGAC (SEQ ID NO:24), or a nucleic acid sequence that hybridizes to a nucleic acid sequence consisting of SEQ ID NO:24 under stringent hybridization conditions.

Also disclosed are polynucleotides comprising nucleic acid sequences encoding an APC-targeting ligand. In some embodiments, the APC-targeting ligand is Galectin-3 and comprises the amino acid sequence: ARSTSSPAAVRSQPTSGKWQTIFRSMMRYLGLETQTLKDGLAHGGTSLLGQGATQGL PILGPTPGRHPQGLILDRHLQAPTLEHLELIPEHLHLESTQGHPAALGPTHLLDSQVPPE PTLPLAPMAPLLGHLCLITCLCLGEWCLACQFWARSPMQTELLISKEGMMLPSTLTHAS MRTTGESLFAIQSWIITGEGKKDSRFSHLKVGNHSKYKYWLNLTTSRLQMMLTCCSTII GLKNSMKSANWEFLVTTSPVLHIPYNLKGADKKKKNLNLTCVKVSCSLVKIFTFINIPLVS HLLNKYYS (SEQ ID NO:25), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:25. Therefore, in some embodiments, the polynucleotide encoding the APC-targeting ligand has the nucleic acid sequence: GCCCGCAGCACCTCCTCGCCAGCAGCCGTCCGGAGCCAGCCAACGAGCGGAAAA TGGCAGACAATTTTTCGCTCCATGATGCGTTATCTGGGTCTGGAAACCCAAACCCT CAAGGATGGCCTGGCGCATGGGGGAACCAGCCTGCTGGGGCAGGGGGCTACCCA GGGGCTTCCTATCCTGGGGCCTACCCCGGGCAGGCACCCCCAGGGGCTTATCCT GGACAGGCACCTCCAGGCGCCTACCCTGGAGCACCTGGAGCTTATCCCGGAGCAC CTGCACCTGGAGTCTACCCAGGGCCACCCAGCGGCCCTGGGGCCTACCCATCTTC TGGACAGCCAAGTGCCACCGGAGCCTACCCTGCCACTGGCCCCTATGGCGCCCCT GCTGGGCCACTGATTGTGCCTTATAACCTGCCTTTGCCTGGGGGAGTGGTGCCTC GCATGCTGATAACAATTCTGGGCACGGTGAAGCCCAATGCAAACAGAATTGCTTTA GATTTCCAAAGAGGGAATGATGTTGCCTTCCACTTTAACCCACGCTTCAATGAGAAC AACAGGAGAGTCATTGTTTGCAATACAAAGCTGGATAATAACTGGGGAAGGGAAGA AAGACAGTCGGTTTTCCCATTTGAAAGTGGGAAACCATTCAAAATACAAGTACTGGT TGAACCTGACCACTTCAAGGTTGCAGTGAATGATGCTCACTTGTTGCAGTACAATCA TCGGGTTAAAAAACTCAATGAAATCAGCAAACTGGGAATTTCTGGTGACATAGACCT CACCAGTGCTTCATATACCATGATATAATCTGAAAGGGGCAGATTAAAAAAAAAAAA AGAATCTAAACCTTACATGTGTAAAGGTTTCATGTTCACTGTGAGTGAAAATTTTTAC ATTCATCAATATCCCTCTTGTAAGTCATCTACTTAATAAATATTACAGTG (SEQ ID NO:26), or a nucleic acid sequence that hybridizes to a nucleic acid sequence consisting of SEQ ID NO:26 under stringent hybridization conditions.

Also disclosed are polynucleotides comprising nucleic acid sequences encoding an APC-targeting ligand. In some embodiments, the APC-targeting ligand is Galectin-1 and comprises the amino acid sequence: ISLGWSLLTAGAPAREHPPGLNHGLWSGRQQPESQTWRVPSSARRGGSRELRAEPG QRQQQPVPALQPSLQRPRRRQHHRVQQQGRRGLGDRAAGGCLSLPAWKCCRGVHH LRPGQPDRQAARWIRIQVPQPPQPGGHQLHGSRLQDQMCGLLKSASPWPPIKAAASA PSE (SEQ ID NO:27), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:27. Therefore, in some embodiments, the polynucleotide encoding the APC-targeting ligand has the nucleic acid sequence: ATCTCTCTCGGGTGGAGTCTTCTGACAGCTGGTGCGCCTGCCCGGGAACATCCTC CTGGACTCAATCATGGCTTGTGGTCTGGTCGCCAGCAACCTGAATCTCAAACCTGG AGAGTGCCTTCGAGTGCGAGGCGAGGTGGCTCCTGACGCTAAGAGCTTCGTGCTG AACCTGGGCAAAGACAGCAACAACCTGTGCCTGCACTTCAACCCTCGCTTCAACGC CCACGGCGACGCCAACACCATCGTGTGCAACAGCAAGGACGGCGGGGCCTGGGG GACCGAGCAGCGGGAGGCTGTCTTTCCCTTCCAGCCTGGAAGTGTTGCAGAGGTG TGCATCACCTTCGACCAGGCCAACCTGACCGTCAAGCTGCCAGATGGATACGAATT CAAGTTCCCCAACCGCCTCAACCTGGAGGCCATCAACTACATGGCAGCTGACGGT GACTTCAAGATCAAATGTGTGGCCTTTGACTGAAATCAGCCAGCCCATGGCCCCCA ATAAAGGCAGCTGCCTCTGCTCCCTCTGAA (SEQ ID NO:28), or a nucleic acid sequence that hybridizes to a nucleic acid sequence consisting of SEQ ID NO:28 under stringent hybridization conditions.

Also disclosed are polynucleotides comprising nucleic acid sequences encoding an APC-targeting ligand. In some embodiments, the APC-targeting ligand is HSP70 and comprises the amino acid sequence: VQLLGFLWLPSLLEPGLHPSNHVQGTCSWYSWHHLLLCGCFPARKSRDNCQSGKPN HSKLCRLYGHTVDRCRKESSCNEPHQHSFCQTSDWTQICCCPVYETLALYGGECWQA QGPSRIQGRDQKLLSRGGVFYGSDKDEGNCRSLPWEDCYQCCGHSASLLLSASGYQ RCWNYCWSQCTNYANCCCYCLRLRQKGWSRKKRAHLPGRWHFCVNPHYGWNLGQ VYSWRHPLGWRRFQPNGQPFYCVAQAEGHQEQESCKTPPYCLTCAYPLFQHPGQYD RFSLRNRLLYLHYPCPIRTECPVPWHPGPSRESPSRCQTRQVTDSYCPGWWFYSYPQ DSEASPRLLQWKRTEEHQPSCCLWCSCPGSHLVWRQVECSRFAALGCHSSFPWYNC WWSHDCPHQAYHHSYQADTDLHYLFQPAWCAYSGLRRACHDKGQPAWQVTHRHTS CTPRCSSDSHFHCQWYTQCLCCGQEYGKREQDYYHQGPFEQGRHTYGPGSEVQSR EAEGQGVIQEFTVLCLQHESNCRETSRQDRGQTEDSGQVNYQLAESDCEGRITSTER AGESLQPHHHQAVPECRRHARRNAWGISWWWSSSLWWCFLRAHHRGLSQPSVDVA LFHTFKTFEGPKFVANSVA (SEQ ID NO:29), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:29. Therefore, in some embodiments, the polynucleotide encoding the APC-targeting ligand has the nucleic acid sequence: GTGCAGCTCTTGGGTTTTTTGTGGCTTCCTTCGTTATTGGAGCCAGGCCTACACCC CAGCAACCATGTCCAAGGGACCTGCAGTTGGTATTGATCTTGGCACCACCTACTCT TGTGTGGGTGTTTTCCAGCACGGAAAAGTCGAGATAATTGCCAATGATCAGGGAAA CCGAACCACTCCAAGCTATGTCGCCTTTACGGACACTGAACGGTTGATCGGTGATG CCGCAAAGAATCAAGTTGCAATGAACCCCACCAACACAGTTTTTGATGCCAAACGT CTGATTGGACGCAGATTTGATGATGCTGTTGTCCAGTCTGATATGAAACATTGGCC CTTTATGGTGGTGAATGATGCTGGCAGGCCCAAGGTCCAAGTAGAATACAAGGGA GAGACCAAAAGCTTCTATCCAGAGGAGGTGTCTTCTATGGTTCTGACAAAGATGAA GGAAATTGCAGAAGCCTACCTTGGGAAGACTGTTACCAATGCTGTGGTCACAGTGC CAGCTTACTTTAATGACTCTCAGCGTCAGGCTACCAAAGATGCTGGAACTATTGCTG GTCTCAATGTACTTAGAATTATTAATGAGCCAACTGCTGCTGCTATTGCTTACGGCT TAGACAAAAAGGTTGGAGCAGAAAGAAACGTGCTCATCTTTGACCTGGGAGGTGGC ACTTTTGATGTGTCAATCCTCACTATTGAGGATGGAATCTTTGAGGTCAAGTCTACA GCTGGAGACACCCACTTGGGTGGAGAAGATTTTGACAACCGAATGGTCAACCATTT TATTGCTGAGTTTAAGCGCAAGCATAAGAAGGACATCAGTGAGAACAAGAGAGCTG TAAGACGCCTCCGTACTGCTTGTGAACGTGCTAAGCGTACCCTCTCTTCCAGCACC CAGGCCAGTATTGAGATCGATTCTCTCTATGAAGGAATCGACTTCTATACCTCCATT ACCCGTGCCCGATTTGAAGAACTGAATGCTGACCTGTTCCGTGGCACCCTGGACC CAGTAGAGAAAGCCCTTCGAGATGCCAAACTAGACAAGTCACAGATTCATGATATT GTCCTGGTTGGTGGTTCTACTCGTATCCCCAAGATTCAGAAGCTTCTCCAAGACTTC TTCAATGGAAAAGAACTGAATAAGAGCATCAACCCTGATGAAGCTGTTGCTTATGGT GCAGCTGTCCAGGCAGCCATCTTGTCTGGAGACAAGTCTGAGAATGTTCAAGATTT GCTGCTCTTGGATGTCACTCCTCTTTCCCTTGGTATTGAAACTGCTGGTGGAGTCAT GACTGTCCTCATCAAGCGTAATACCACCATTCCTACCAAGCAGACACAGACCTTCA CTACCTATTCTGACAACCAGCCTGGTGTGCTTATTCAGGTTTATGAAGGCGAGCGT GCCATGACAAAGGATAACAACCTGCTTGGCAAGTTTGAACTCACAGGCATACCTCC TGCACCCCGAGGTGTTCCTCAGATTGAAGTCACTTTTGACATTGATGCCAATGGTAT ACTCAATGTCTCTGCTGTGGACAAGAGTACGGGAAAAGAGAACAAGATTACTATCA CTAATGACAAGGGCCGTTTGAGCAAGGAAGACATTGAACGTATGGTCCAGGAAGCT GAGAAGTACAAAGCTGAAGATGAGAAGCAGAGGGACAAGGTGTCATCCAAGAATTC ACTTGAGTCCTATGCCTTCAACATGAAAGCAACTGTTGAAGATGAGAAACTTCAAGG CAAGATTAACGATGAGGACAAACAGAAGATTCTGGACAAGTGTAATGAAATTATCAA CTGGCTTGATAAGAATCAGACTGCTGAGAAGGAAGAATTTGAACATCAACAGAAAG AGCTGGAGAAAGTTTGCAACCCCATCATCACCAAGCTGTACCAGAGTGCAGGAGG CATGCCAGGAGGAATGCCTGGGGGATTTCCTGGTGGTGGAGCTCCTCCCTCTGGT GGTGCTTCCTCAGGGCCCACCATTGAAGAGGTTGATTAAGCCAACCAAGTGTAGAT GTAGCATTGTTCCACACATTTAAAACATTTGAAGGACCTAAATTCGTAGCAAATTCT GTGGCA (SEQ ID NO:30), or a nucleic acid sequence that hybridizes to a nucleic acid sequence consisting of SEQ ID NO:30 under stringent hybridization conditions.

### Transmembrane Domain

Also disclosed are polynucleotides comprising nucleic acid sequences encoding a transmembrane protein suitable for guiding the APC-targeting ligand into an exosome. Examples of this type of proteins include tetraspanins CD9, CD63, and CD81.

Therefore, in some embodiments, the transmembrane protein is CD9 and comprises the amino acid sequence: DQPTAACICIQRQVPPVPAARAPQSRTRSAQAKLALTMPVKGGTKCIKYLLFGFNFIFW LAGIAVLAIGLWLRFDSQTKSIFEQETNNNNSSFYTGVYILIGAGALMMLVGFLGCCGAV QESQCMLGLFFGFLLVIFAIEIAAAIWGYSHKDEVIKEVQEFYKDTYNKLKTKDEPQRETL KAIHYALNCCGLAGGVEQFISDICPKKDVLETFTVKSCPDAIKEVFDNKFHIIGAVGIGIAV VMIFGMIFSMILCCAIRRNREMVSQLTSLSRKVYPRLVGFFVCLFCFVCCLLFVFLPLILV FILHCIKAEVTLCLSFNASFNIDICSAGGLVCFGLYFFSCLFLLVILSRNPAMKGTIFARLT RYCTKNFFVFKIQMSINFNQVVTYIEDNLIHNKKLQCQKKKK (SEQ ID NO:31), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:31.

Therefore, in some embodiments, the polynucleotide encoding the APC-targeting ligand has the nucleic acid sequence: GACCAGCCTACAGCCGCCTGCATCTGTATCCAGCGCCAGGTCCCGCCAGTCCCAG CTGCGCGCGCCCCCCAGTCCCGCACCCGTTCGGCCCAGGCTAAGTTAGCCCTCAC CATGCCGGTCAAAGGAGGCACCAAGTGCATCAAATACCTGCTGTTCGGATTTAACT TCATCTTCTGGCTTGCCGGGATTGCTGTCCTTGCCATTGGACTATGGCTCCGATTC GACTCTCAGACCAAGAGCATCTTCGAGCAAGAAACTAATAATAATAATTCCAGCTTC TACACAGGAGTCTATATTCTGATCGGAGCCGGCGCCCTCATGATGCTGGTGGGCTT CCTGGGCTGCTGCGGGGCTGTGCAGGAGTCCCAGTGCATGCTGGGACTGTTCTTC GGCTTCCTCTTGGTGATATTCGCCATTGAAATAGCTGCGGCCATCTGGGGATATTC CCACAAGGATGAGGTGATTAAGGAAGTCCAGGAGTTTTACAAGGACACCTACAACA AGCTGAAAACCAAGGATGAGCCCCAGCGGGAAACGCTGAAAGCCATCCACTATGC GTTGAACTGCTGTGGTTTGGCTGGGGGCGTGGAACAGTTTATCTCAGACATCTGCC CCAAGAAGGACGTACTCGAAACCTTCACCGTGAAGTCCTGTCCTGATGCCATCAAA GAGGTCTTCGACAATAAATTCCACATCATCGGCGCAGTGGGCATCGGCATTGCCGT GGTCATGATATTTGGCATGATCTTCAGTATGATCTTGTGCTGTGCTATCCGCAGGAA CCGCGAGATGGTCTAGAGTCAGCTTACATCCCTGAGCAGGAAAGTTTACCCATGAA GATTGGTGGGATTTTTTGTTTGTTTGTTTTGTTTTGTTTGTTGTTTGTTGTTTGTTTTT TTGCCACTAATTTTAGTATTCATTCTGCATTGCTAGATAAAAGCTGAAGTTACTTTAT GTTTGTCTTTTAATGCTTCATTCAATATTGACATTTGTAGTTGAGCGGGGGGTTTGG TTTGCTTTGGTTTATATTTTTTCAGTTGTTTGTTTTTGCTTGTTATATTAAGCAGAAAT CCTGCAATGAAAGGTACTATATTTGCTAGACTCTAGACAAGATATTGTACATAAAAG AATTTTTTTGTCTTTAAATAGATACAAATGTCTATCAACTTTAATCAAGTTGTAACTTA TATTGAAGACAATTTGATACATAATAAAAAATTATGACAATGTCAAAAAAAAAAAAAA A (SEQ ID NO:32), or a nucleic acid sequence that hybridizes to a nucleic acid sequence consisting of SEQ ID NO:32 under stringent hybridization conditions.

In some embodiments, the transmembrane protein is CD63 and comprises the amino acid sequence: MAVEGGMKCVKFLLYVLLLAFCACAVGLIAVGVGAQLVLSQTIIQGATPGSLLPVVIIAVG VFLFLVAFVGCCGACKENYCLMITFAIFLSLIMLVEVAAAIAGYVFRDKVMSEFNNNFRQ QMENYPKNNHTASILDRMQADFKCCGAANYTDWEKIPSMSKNRVPDSCCINVTVGCGI NFNEKAIHKEGCVEKIGGWLRKNVLVVAAAALGIAFVEVLGIVFACCLVKSIRSGYEVM (SEQ ID NO:33), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:33. Therefore, in some embodiments, the polynucleotide encoding the APC-targeting ligand has the nucleic acid sequence:

(SEQ ID NO: 34)
ATGGCGGTGGAAGGAGGAATGAAATGTGTGAAGTTCTTGCTCTACGTCC
TCCTGCTGGCCTTTTGCGCCTGTGCAGTGGGACTGATTGCCGTGGGTGT
CGGGGCACAGCTTGTCCTGAGTCAGACCATAATCCAGGGGGCTACCCCT
GGCTCTCTGTTGCCAGTGGTCATCATCGCAGTGGGTGTCTTCCTCTTCC
TGGTGGCTTTTGTGGGCTGCTGCGGGGCCTGCAAGGAGAACTATTGTCT
TATGATCACGTTTGCCATCTTTCTGTCTCTTATCATGTTGGTGGAGGTG
GCCGCAGCCATTGCTGGCTATGTGTTTAGAGATAAGGTGATGTCAGAGT
TTAATAACAACTTCCGGCAGCAGATGGAGAATTACCCGAAAAACAACCA
CACTGCTTCGATCCTGGACAGGATGCAGGCAGATTTTAAGTGCTGTGGG
GCTGCTAACTACACAGATTGGGAGAAAATCCCTTCCATGTCGAAGAACC
GAGTCCCCGACTCCTGCTGCATTAATGTTACTGTGGGCTGTGGGATTAA
TTTCAACGAGAAGGCGATCCATAAGGAGGGCTGTGTGGAGAAGATTGGG
GGCTGGCTGAGGAAAAATGTGCTGGTGGTAGCTGCAGCAGCCCTTGGAA
TTGCTTTTGTCGAGGTTTTGGGAATTGTCTTTGCCTGCTGCCTCGTGAA
GAGTATCAGAAGTGGCTACGAGGTGATG,

or a nucleic acid sequence that hybridizes to a nucleic acid sequence consisting of SEQ ID NO:34 under stringent hybridization conditions.

Therefore, in some embodiments, the transmembrane protein is CD81 and comprises the amino acid sequence: GQRASAQRRRRRRPHRASCQASGAQRPTRPRAPAPPRPFLRAPAPRPARPPCRPPA RPRAGPPAAQDRPAPRRPPAARAAMGVEGCTKCIKYLLFVFNFVFWLAGGVILGVALW LRHDPQTTNLLYLELGDKPAPNTFYVGIYILIAVGAVMMFVGFLGCYGAIQESQCLLGTF FTCLVILFACEVAAGIWGFVNKDQIAKDVKQFYDQALQQAVVDDDANNAKAVVKTFHET LDCCGSSTLTALTTSVLKNNLCPSGSNIISNLFKEDCHQKIDDLFSGKLYLIGIAAIVVAVI MIFEMILSMVLCCGIRNSSVYGPAALATGTSAVPPKPGHFRGGHHRLCIRFRYYSATRS LFTFGVLFLFTFLLPFQGRHMVACMSGDGPGSWGLEGRGPSALGSQGALPAQPGLS WEPLAQRLSLANLGGCVHPARPSCGLHSSPCSLLPRFESRVCGHSLPSCTCPFHVAF NCNHNILTPSFNKEGTSGML (SEQ ID NO:35), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:35. Therefore, in some embodiments, the polynucleotide encoding the APC-targeting ligand has the nucleic acid sequence: GGCCAGAGAGCGAGCGCGCAACGGCGGCGACGGCGGCGACCCCACCGCGCATC CTGCCAGGCCTCCGGCGCCCAGCGCCCCACGCGCCCCCGCGCCCCCGCGCCCC CGCGCCCCTTTCTTCGCGCCCCCGCCCCTCGGCCCGCCAGGCCCCCTTGCCGGC CACCCGCCAGGCCCCGCGCCGGCCCGCCCGCCGCCCAGGACCGGCCCGCGCCC CGCAGGCCGCCCGCCGCCCGCGCCGCCATGGGAGTGGAGGGCTGCACCAAGTGC ATCAAGTACCTGCTCTTCGTCTTCAATTTCGTCTTCTGGCTGGCTGGAGGCGTGATC CTGGGTGTGGCCCTGTGGCTCCGCCATGACCCGCAGACCACCAACCTCCTGTATCT GGAGCTGGGAGACAAGCCCGCGCCCAACACCTTCTATGTAGGCATCTACATCCTCA TCGCTGTGGGCGCTGTCATGATGTTCGTTGGCTTCCTGGGCTGCTACGGGGCCATC CAGGAATCCCAGTGCCTGCTGGGGACGTTCTTCACCTGCCTGGTCATCCTGTTTGC CTGTGAGGTGGCCGCCGGCATCTGGGGCTTTGTCAACAAGGACCAGATCGCCAAG GATGTGAAGCAGTTCTATGACCAGGCCCTACAGCAGGCCGTGGTGGATGATGACGC CAACAACGCCAAGGCTGTGGTGAAGACCTTCCACGAGACGCTTGACTGCTGTGGC TCCAGCACACTGACTGCTTTGACCACCTCAGTGCTCAAGAACAATTTGTGTCCCTCG GGCAGCAACATCATCAGCAACCTCTTCAAGGAGGACTGCCACCAGAAGATCGATGA CCTCTTCTCCGGGAAGCTGTACCTCATCGGCATTGCTGCCATCGTGGTCGCTGTGA TCATGATCTTCGAGATGATCCTGAGCATGGTGCTGTGCTGTGGCATCCGGAACAGC TCCGTGTACTGAGGCCCCGCAGCTCTGGCCACAGGGACCTCTGCAGTGCCCCCTA AGTGACCCGGACACTTCCGAGGGGGCCATCACCGCCTGTGTATATAACGTTTCCGG TATTACTCTGCTACACGTAGCCTTTTTACTTTTGGGGTTTTGTTTTTGTTCTGAACTTT CCTGTTACCTTTTCAGGGCTGACGTCACATGTAGGTGGCGTGTATGAGTGGAGACG GGCCTGGGTCTTGGGGACTGGAGGGCAGGGGTCCTTCTGCCCTGGGGTCCCAGG GTGCTCTGCCTGCTCAGCCAGGCCTCTCCTGGGAGCCACTCGCCCAGAGACTCAG CTTGGCCAACTTGGGGGGCTGTGTCCACCCAGCCCGCCCGTCCTGTGGGCTGCAC AGCTCACCTTGTTCCCTCCTGCCCCGGTTCGAGAGCCGAGTCTGTGGGCACTCTCT GCCTTCATGCACCTGTCCTTTCTAACACGTCGCCTTCAACTGTAATCACAACATCCT GACTCCGTCATTTAATAAAGAAGGAACATCAGGCATGCTA (SEQ ID NO:36), or a nucleic acid sequence that hybridizes to a nucleic acid sequence consisting of SEQ ID NO:36 under stringent hybridization conditions.

### Example Fusion Proteins

In some embodiments, the fusion protein contains SARS-COV2 spike protein+Poly His tag+CD63 and therefore can comprise the amino acid sequence: MFVFLVLLPLVSSQCVNLTTRTQLPPAYTNSFTRGVYYPDKVFRSSVLHSTQDLFLPFF SNVTWFHAIHVSGTNGTKRFDNPVLPFNDGVYFASTEKSNIIRGWIFGTTLDSKTQSLLI VNNATNVVIKVCEFQFCNDPFLGVYYHKNNKSWMESEFRVYSSANNCTFEYVSQPFL MDLEGKQGNFKNLREFVFKNIDGYFKIYSKHTPINLVRDLPQGFSALEPLVDLPIGINITR FQTLLALHRSYLTPGDSSSGWTAGAAAYYVGYLQPRTFLLKYNENGTITDAVDCALDPL SETKCTLKSFTVEKGIYQTSNFRVQPTESIVRFPNITNLCPFGEVFNATRFASVYAWNRK RISNCVADYSVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFERDISTEIYQ AGSTPCNGVEGFNCYFPLQSYGFQPTNGVGYQPYRVVVLSFELLHAPATVCGPKKST NLVKNKCVNFNFNGLTGTGVLTESNKKFLPFQQFGRDIADTTDAVRDPQTLEILDITPCS FGGVSVITPGTNTSNQVAVLYQDVNCTEVPVAIHADQLTPTWRVYSTGSNVFQTRAGC LIGAEHVNNSYECDIPIGAGICASYQTQTNSPRRARSVASQSIIAYTMSLGAENSVAYSN NSIAIPTNFTISVTTEILPVSMTKTSVDCTMYICGDSTECSNLLLQYGSFCTQLNRALTGIA VEQDKNTQEVFAQVKQIYKTPPIKDFGGFNFSQILPDPSKPSKRSFIEDLLFNKVTLADA GFIKQYGDCLGDIAARDLICAQKFNGLTVLPPLLTDEMIAQYTSALLAGTITSGWTFGAG AALQIPFAMQMAYRFNGIGVTQNVLYENQKLIANQFNSAIGKIQDSLSSTASALGKLQDV VNQNAQALNTLVKQLSSNFGAISSVLNDILSRLDKVEAEVQIDRLITGRLQSLQTYVTQQ LIRAAEIRASANLAATKMSECVLGQSKRVDFCGKGYHLMSFPQSAPHGVVFLHVTYVP AQEKNFTTAPAICHDGKAHFPREGVFVSNGTHWFVTQRNFYEPQIITTDNTFVSGNCD VVIGIVNNTVYDPLQPELDSFKEELDKYFKNHTSPDVDLGDISGINASVVNIQKEIDRLNE VAKNLNESLIDLQELGKYEQYIKWPWYIWLGFIAGLIAIVMVTIMLCCMTSCCSCLKGCC SCGSCCKFDEDDSEPVLKGVKLHYTHHHHHHMAVEGGMKCVKFLLYVLLLAFCACAV GLIAVGVGAQLVLSQTIIQGATPGSLLPVVIAVGVFLFLVAFVGCCGACKENYCLMITFAI FLSLIMLVEVAAAIAGYVFRDKVMSEFNNNFRQQMENYPKNNHTASILDRMQADFKCC GAANYTDWEKIPSMSKNRVPDSCCINVTVGCGINFNEKAIHKEGCVEKIGGWLRKNVLV VAAAALGIAFVEVLGIVFACCLVKSIRSGYEVM (SEQ ID NO:41), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:41. Therefore, in some embodiments, the fusion protein is encoded by the nucleic acid sequence: ATGTTTGTTTTTCTTGTTTTATTGCCACTAGTCTCTAGTCAGTGTGTTAATCTTACAA CCAGAACTCAATTACCCCCTGCATACACTAATTCTTTCACACGTGGTGTTTATTACC CTGACAAAGTTTTCAGATCCTCAGTTTTACATTCAACTCAGGACTTGTTCTTACCTTT CTTTTCCAATGTTACTTGGTTCCATGCTATACATGTCTCTGGGACCAATGGTACTAA GAGGTTTGATAACCCTGTCCTACCATTTAATGATGGTGTTTATTTTGCTTCCACTGA GAAGTCTAACATAATAAGAGGCTGGATTTTTGGTACTACTTTAGATTCGAAGACCCA GTCCCTACTTATTGTTAATAACGCTACTAATGTTGTTATTAAAGTCTGTGAATTTCAA TTTTGTAATGATCCATTTTTGGGTGTTTATTACCACAAAAACAACAAAAGTTGGATGG AAAGTGAGTTCAGAGTTTATTCTAGTGCGAATAATTGCACTTTTGAATATGTCTCTCA GCCTTTTCTTATGGACCTTGAAGGAAAACAGGGTAATTTCAAAAATCTTAGGGAATT TGTGTTTAAGAATATTGATGGTTATTTTAAAATATATTCTAAGCACACGCCTATTAATT TAGTGCGTGATCTCCCTCAGGGTTTTTCGGCTTTAGAACCATTGGTAGATTTGCCAA TAGGTATTAACATCACTAGGTTTCAAACTTTACTTGCTTTACATAGAAGTTATTTGAC TCCTGGTGATTCTTCTTCAGGTTGGACAGCTGGTGCTGCAGCTTATTATGTGGGTTA TCTTCAACCTAGGACTTTTCTATTAAAATATAATGAAAATGGAACCATTACAGATGCT GTAGACTGTGCACTTGACCCTCTCTCAGAAACAAAGTGTACGTTGAAATCCTTCACT GTAGAAAAAGGAATCTATCAAACTTCTAACTTTAGAGTCCAACCAACAGAATCTATT GTTAGATTTCCTAATATTACAAACTTGTGCCCTTTTGGTGAAGTTTTTAACGCCACCA GATTTGCATCTGTTTATGCTTGGAACAGGAAGAGAATCAGCAACTGTGTTGCTGATT ATTCTGTCCTATATAATTCCGCATCATTTTCCACTTTTAAGTGTTATGGAGTGTCTCC TACTAAATTAAATGATCTCTGCTTTACTAATGTCTATGCAGATTCATTTGTAATTAGA GGTGATGAAGTCAGACAAATCGCTCCAGGGCAAACTGGAAAGATTGCTGATTATAA TTATAAATTACCAGATGATTTTACAGGCTGCGTTATAGCTTGGAATTCTAACAATCTT GATTCTAAGGTTGGTGGTAATTATAATTACCTGTATAGATTGTTTAGGAAGTCTAATC TCAAACCTTTTGAGAGAGATATTTCAACTGAAATCTATCAGGCCGGTAGCACACCTT GTAATGGTGTTGAAGGTTTTAATTGTTACTTTCCTTTACAATCATATGGTTTCCAACC CACTAATGGTGTTGGTTACCAACCATACAGAGTAGTAGTACTTTCTTTTGAACTTCTA CATGCACCAGCAACTGTTTGTGGACCTAAAAAGTCTACTAATTTGGTTAAAAACAAA TGTGTCAATTTCAACTTCAATGGTTTAACAGGCACAGGTGTTCTTACTGAGTCTAAC AAAAAGTTTCTGCCTTTCCAACAATTTGGCAGAGACATTGCTGACACTACTGATGCT GTCCGTGATCCACAGACACTTGAGATTCTTGACATTACACCATGTTCTTTTGGTGGT GTCAGTGTTATAACACCAGGAACAAATACTTCTAACCAGGTTGCTGTTCTTTATCAG GATGTTAACTGCACAGAAGTCCCTGTTGCTATTCATGCAGATCAACTTACTCCTACT TGGCGTGTTTATTCTACAGGTTCTAATGTTTTTCAAACACGTGCAGGCTGTTTAATA GGGGCTGAACATGTCAACAACTCATATGAGTGTGACATACCCATTGGTGCAGGTAT ATGCGCTAGTTATCAGACTCAGACTAATTCTCCTCGGCGGGCACGTAGTGTAGCTA GTCAATCCATCATTGCCTACACTATGTCACTTGGTGCAGAAAATTCAGTTGCTTACT CTAATAACTCTATTGCCATACCCACAAATTTTACTATTAGTGTTACCACAGAAATTCT ACCAGTGTCTATGACCAAGACATCAGTAGATTGTACAATGTACATTTGTGGTGATTC AACTGAATGCAGCAATCTTTTGTTGCAATATGGCAGTTTTTGTACACAATTAAACCGT GCTTTAACTGGAATAGCTGTTGAACAAGACAAAAACACCCAAGAAGTTTTTGCACAA GTCAAACAAATTTACAAAACACCACCAATTAAAGATTTTGGTGGTTTTAATTTTTCAC AAATATTACCAGATCCATCAAAACCAAGCAAGAGGTCATTTATTGAAGATCTACTTTT CAACAAAGTGACACTTGCAGATGCTGGCTTCATCAAACAATATGGTGATTGCCTTG GTGATATTGCTGCTAGAGACCTCATTTGTGCACAAAAGTTTAACGGCCTTACTGTTT TGCCACCTTTGCTCACAGATGAAATGATTGCTCAATACACTTCTGCACTGTTAGCGG GTACAATCACTTCTGGTTGGACCTTTGGTGCAGGTGCTGCATTACAAATACCATTTG CTATGCAAATGGCTTATAGGTTTAATGGTATTGGAGTTACACAGAATGTTCTCTATG AGAACCAAAAATTGATTGCCAACCAATTTAATAGTGCTATTGGCAAAATTCAAGACT CACTTTCTTCCACAGCAAGTGCACTTGGAAAACTTCAAGATGTGGTCAACCAAAATG CACAAGCTTTAAACACGCTTGTTAAACAACTTAGCTCCAATTTTGGTGCAATTTCAA GTGTTTTAAATGATATCCTTTCACGTCTTGACAAAGTTGAGGCTGAAGTGCAAATTG ATAGGTTGATCACAGGCAGACTTCAAAGTTTGCAGACATATGTGACTCAACAATTAA TTAGAGCTGCAGAAATCAGAGCTTCTGCTAATCTTGCTGCTACTAAAATGTCAGAGT GTGTACTTGGACAATCAAAAAGAGTTGATTTTTGTGGAAAGGGCTATCATCTTATGT CCTTCCCTCAGTCAGCACCTCATGGTGTAGTCTTCTTGCATGTGACTTATGTCCCTG CACAAGAAAAGAACTTCACAACTGCTCCTGCCATTTGTCATGATGGAAAAGCACACT TTCCTCGTGAAGGTGTCTTTGTTTCAAATGGCACACACTGGTTTGTAACACAAAGGA ATTTTTATGAACCACAAATCATTACTACAGACAACACATTTGTGTCTGGTAACTGTGA TGTTGTAATAGGAATTGTCAACAACACAGTTTATGATCCTTTGCAACCTGAATTAGA CTCATTCAAGGAGGAGTTAGATAAATATTTTAAGAATCATACATCACCAGATGTTGAT TTAGGTGACATCTCTGGCATTAATGCTTCAGTTGTAAACATTCAAAAAGAAATTGAC CGCCTCAATGAGGTTGCCAAGAATTTAAATGAATCTCTCATCGATCTCCAAGAACTT GGAAAGTATGAGCAGTATATAAAATGGCCATGGTACATTTGGCTAGGTTTTATAGCT GGCTTGATTGCCATAGTAATGGTGACAATTATGCTTTGCTGTATGACCAGTTGCTGT AGTTGTCTCAAGGGCTGTTGTTCTTGTGGATCCTGCTGCAAATTTGATGAAGACGA CTCTGAGCCAGTGCTCAAAGGAGTCAAATTACATTACACCCACCACCACCACCACC ACATGGCGGTGGAAGGAGGAATGAAATGTGTGAAGTTCTTGCTCTACGTCCTCCTG CTGGCCTTTTGCGCCTGTGCAGTGGGACTGATTGCCGTGGGTGTCGGGGCACAGC TTGTCCTGAGTCAGACCATAATCCAGGGGGCTACCCCTGGCTCTCTGTTGCCAGTG GTCATCATCGCAGTGGGTGTCTTCCTCTTCCTGGTGGCTTTTGTGGGCTGCTGCGG GGCCTGCAAGGAGAACTATTGTCTTATGATCACGTTTGCCATCTTTCTGTCTCTTAT CATGTTGGTGGAGGTGGCCGCAGCCATTGCTGGCTATGTGTTTAGAGATAAGGTGA TGTCAGAGTTTAATAACAACTTCCGGCAGCAGATGGAGAATTACCCGAAAAACAAC CACACTGCTTCGATCCTGGACAGGATGCAGGCAGATTTTAAGTGCTGTGGGGCTG CTAACTACACAGATTGGGAGAAAATCCCTTCCATGTCGAAGAACCGAGTCCCCGAC TCCTGCTGCATTAATGTTACTGTGGGCTGTGGGATTAATTTCAACGAGAAGGCGAT CCATAAGGAGGGCTGTGTGGAGAAGATTGGGGGCTGGCTGAGGAAAAATGTGCTG GTGGTAGCTGCAGCAGCCCTTGGAATTGCTTTTGTCGAGGTTTTGGGAATTGTCTT TGCCTGCTGCCTCGTGAAGAGTATCAGAAGTGGCTACGAGGTGATG (SEQ ID NO:42), or a nucleic acid sequence that hybridizes to a nucleic acid sequence consisting of SEQ ID NO:42 under stringent hybridization conditions.

In some embodiments, the fusion protein contains Her-2/neu+FLAG+CD81 and therefore can comprise the amino acid sequence: KGR-PWPLWSGPRAAARPFPRGPLLRRAPGPHPSQHPAPRALPAGSSRSHGAGAAVSTME LAALCRWGLLLALLPPGAASTQVCTGTDMKLRLPASPETHLDMLRHLYQGCQVVQGNL ELTYLPTNASLSFLQDIQEVQGYVLIAHNQVRQVPLQRLRIVRGTQLFEDNYALAVLDNG DPLNNTTPVTGASPGGLRELQLRSLTEILKGGVLIQRNPQLCYQDTILWKDIFHKNNQLA LTLIDTNRSRACHPCSPMCKGSRCWGESSEDCQSLTRTVCAGGCARCKGPLPTDCCH EQCAAGCTGPKHSDCLACLHFNHSGICELHCPALVTYNTDTFESMPNPEGRYTFGASC VTACPYNYLSTDVGSCTLVCPLHNQEVTAEDGTQRCEKCSKPCARVCYGLGMEHLRE VRAVTSANIQEFAGCKKIFGSLAFLPESFDGDPASNTAPLQPEQLQVFETLEEITGYLYIS AWPDSLPDLSVFQNLQVIRGRILHNGAYSLTLQGLGISWLGLRSLRELGSGLALIHHNTH LCFVHTVPWDQLFRNPHQALLHTANRPEDECVGEGLACHQLCARGHCWGPGPTQCV NCSQFLRGQECVEECRVLQGLPREYVNARHCLPCHPECQPQNGSVTCFGPEADQCV ACAHYKDPPFCVARCPSGVKPDLSYMPIWKFPDEEGACQPCPINCTHSCVDLDDKGC PAEQRASPLTSIISAVVGILLVVVLGVVFGILIKRRQQKIRKYTMRRLLQETELVEPLTPSG AMPNQAQMRILKETELRKVKVLGSGAFGTVYKGIWIPDGENVKIPVAIKVLRENTSPKAN KEILDEAYVMAGVGSPYVSRLLGICLTSTVQLVTQLMPYGCLLDHVRENRGRLGSQDLL NWCMQIAKGMSYLEDVRLVHRDLAARNVLVKSPNHVKITDFGLARLLDIDETEYHADG GKVPIKWMALESILRRRFTHQSDVWSYGVTVWELMTFGAKPYDGIPAREIPDLLEKGE RLPQPPICTIDVYMIMVKCWMIDSECRPRFRELVSEFSRMARDPQRFVVIQNEDLGPAS PLDSTFYRSLLEDDDMGDLVDAEEYLVPQQGFFCPDPAPGAGGMVHHRHRSSSTRSG GGDLTLGLEPSEEEAPRSPLAPSEGAGSDVFDGDLGMGAAKGLQSLPTHDPSPLQRY SEDPTVPLPSETDGYVAPLTCSPQPEYVNQPDVRPQPPSPREGPLPAARPAGATLERP KTLSPGKNGVVKDVFAFGGAVENPEYLTPQGGAAPQPHPPPAFSPAFDNLYYWDQDP PERGAPPSTFKGTPTAENPEYLGLDVPVTRRPSPQKPCVLREQGRPDFCWHQEVGGP SDHFQGNLPCQEPVLRNLPSCLSSQMAGRGPASLEEEQHWGVFVDSEALPNETLGSS GCHSPAWPFPSRSWVLKALGKLAEGKRPGSVEQKRPIQRLSLKPSTAPHEEGTAMVS VSRLCTECFSVFLLFLFCFFKDEIKTQGEDYKDDDDKGQRASAQRRRRRRPHRASCQA SGAQRPTRPRAPAPPRPFLRAPAPRPARPPCRPPARPRAGPPAAQDRPAPRRPPAAR AAMGVEGCTKCIKYLLFVFNFVFWLAGGVILGVALWLRHDPQTTNLLYLELGDKPAPNT FYVGIYILIAVGAVMMFVGFLGCYGAIQESQCLLGTFFTCLVILFACEVAAGIWGFVNKD QIAKDVKQFYDQALQQAVVDDDANNAKAVVKTFHETLDCCGSSTLTALTTSVLKNNLC PSGSNIISNLFKEDCHQKIDDLFSGKLYLIGIAAIVVAVIMIFEMILSMVLCCGIRNSSVYGP AALATGTSAVPPKPGHFRGGHHRLCIRFRYYSATRSLFTFGVLFLFTFLLPFQGRHMVA CMSGDGPGSWGLEGRGPSALGSQGALPAQPGLSWEPLAQRLSLANLGGCVHPARPS CGLHSSPCSLLPRFESRVCGHSLPSCTCPF-HVAFNCNHNILTPSFNKEGTSGML (SEQ ID NO:43), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:43. Therefore, in some embodiments, the fusion protein is encoded by the nucleic acid sequence: AAGGGGAGGTAACCCTGGCCCCTTTGGTCGGGGCCCCGGGCAGCCGCGCGCCCC TTCCCACGGGGCCCTTTACTGCGCCGCGCGCCCGGCCCCCACCCCTCGCAGCAC CCCGCGCCCCGCGCCCTCCCAGCCGGGTCCAGCCGGAGCCATGGGGCCGGAGC CGCAGTGAGCACCATGGAGCTGGCGGCCTTGTGCCGCTGGGGGCTCCTCCTCGC CCTCTTGCCCCCCGGAGCCGCGAGCACCCAAGTGTGCACCGGCACAGACATGAAG CTGCGGCTCCCTGCCAGTCCCGAGACCCACCTGGACATGCTCCGCCACCTCTACC AGGGCTGCCAGGTGGTGCAGGGAAACCTGGAACTCACCTACCTGCCCACCAATGC CAGCCTGTCCTTCCTGCAGGATATCCAGGAGGTGCAGGGCTACGTGCTCATCGCT CACAACCAAGTGAGGCAGGTCCCACTGCAGAGGCTGCGGATTGTGCGAGGCACCC AGCTCTTTGAGGACAACTATGCCCTGGCCGTGCTAGACAATGGAGACCCGCTGAA CAATACCACCCCTGTCACAGGGGCCTCCCCAGGAGGCCTGCGGGAGCTGCAGCTT CGAAGCCTCACAGAGATCTTGAAAGGAGGGGTCTTGATCCAGCGGAACCCCCAGC TCTGCTACCAGGACACGATTTTGTGGAAGGACATCTTCCACAAGAACAACCAGCTG GCTCTCACACTGATAGACACCAACCGCTCTCGGGCCTGCCACCCCTGTTCTCCGAT GTGTAAGGGCTCCCGCTGCTGGGGAGAGAGTTCTGAGGATTGTCAGAGCCTGACG CGCACTGTCTGTGCCGGTGGCTGTGCCCGCTGCAAGGGGCCACTGCCCACTGACT GCTGCCATGAGCAGTGTGCTGCCGGCTGCACGGGCCCCAAGCACTCTGACTGCCT GGCCTGCCTCCACTTCAACCACAGTGGCATCTGTGAGCTGCACTGCCCAGCCCTG GTCACCTACAACACAGACACGTTTGAGTCCATGCCCAATCCCGAGGGCCGGTATAC ATTCGGCGCCAGCTGTGTGACTGCCTGTCCCTACAACTACCTTTCTACGGACGTGG GATCCTGCACCCTCGTCTGCCCCCTGCACAACCAAGAGGTGACAGCAGAGGATGG AACACAGCGGTGTGAGAAGTGCAGCAAGCCCTGTGCCCGAGTGTGCTATGGTCTG GGCATGGAGCACTTGCGAGAGGTGAGGGCAGTTACCAGTGCCAATATCCAGGAGT TTGCTGGCTGCAAGAAGATCTTTGGGAGCCTGGCATTTCTGCCGGAGAGCTTTGAT GGGGACCCAGCCTCCAACACTGCCCCGCTCCAGCCAGAGCAGCTCCAAGTGTTTG AGACTCTGGAAGAGATCACAGGTTACCTATACATCTCAGCATGGCCGGACAGCCTG CCTGACCTCAGCGTCTTCCAGAACCTGCAAGTAATCCGGGGACGAATTCTGCACAA TGGCGCCTACTCGCTGACCCTGCAAGGGCTGGGCATCAGCTGGCTGGGGCTGCG CTCACTGAGGGAACTGGGCAGTGGACTGGCCCTCATCCACCATAACACCCACCTC TGCTTCGTGCACACGGTGCCCTGGGACCAGCTCTTTCGGAACCCGCACCAAGCTC TGCTCCACACTGCCAACCGGCCAGAGGACGAGTGTGTGGGCGAGGGCCTGGCCT GCCACCAGCTGTGCGCCCGAGGGCACTGCTGGGGTCCAGGGCCCACCCAGTGTG TCAACTGCAGCCAGTTCCTTCGGGGCCAGGAGTGCGTGGAGGAATGCCGAGTACT GCAGGGGCTCCCCAGGGAGTATGTGAATGCCAGGCACTGTTTGCCGTGCCACCCT GAGTGTCAGCCCCAGAATGGCTCAGTGACCTGTTTTGGACCGGAGGCTGACCAGT GTGTGGCCTGTGCCCACTATAAGGACCCTCCCTTCTGCGTGGCCCGCTGCCCCAG CGGTGTGAAACCTGACCTCTCCTACATGCCCATCTGGAAGTTTCCAGATGAGGAGG GCGCATGCCAGCCTTGCCCCATCAACTGCACCCACTCCTGTGTGGACCTGGATGA CAAGGGCTGCCCCGCCGAGCAGAGAGCCAGCCCTCTGACGTCCATCATCTCTGCG GTGGT TGGCATTCTGCTGGTCGTGGTCTTGGGGGTGGTCTTTGGGATCCTCATCAA GCGACGGCAGCAGAAGATCCGGAAGTACACGATGCGGAGACTGCTGCAGGAAAC GGAGCTGGTGGAGCCGCTGACACCTAGCGGAGCGATGCCCAACCAGGCGCAGAT GCGGATCCTGAAAGAGACGGAGCTGAGGAAGGTGAAGGTGCTTGGATCTGGCGCT TTTGGCACAGTCTACAAGGGCATCTGGATCCCTGATGGGGAGAATGTGAAAATTCC AGTGGCCATCAAAGTGTTGAGGGAAAACACATCCCCCAAAGCCAACAAAGAAATCT TAGACGAAGCATACGTGATGGCTGGTGTGGGCTCCCCATATGTCTCCCGCCTTCTG GGCATCTGCCTGACATCCACGGTGCAGCTGGTGACACAGCTTATGCCCTATGGCT GCCTCTTAGACCATGTCCGGGAAAACCGCGGACGCCTGGGCTCCCAGGACCTGCT GAACTGGTGTATGCAGATTGCCAAGGGGATGAGCTACCTGGAGGATGTGCGGCTC GTACACAGGGACTTGGCCGCTCGGAACGTGCTGGTCAAGAGTCCCAACCATGTCA AAATTACAGACTTCGGGCTGGCTCGGCTGCTGGACATTGACGAGACAGAGTACCAT GCAGATGGGGGCAAGGTGCCCATCAAGTGGATGGCGCTGGAGTCCATTCTCCGCC GGCGGTTCACCCACCAGAGTGATGTGTGGAGTTATGGTGTGACTGTGTGGGAGCT GATGACTTTTGGGGCCAAACCTTACGATGGGATCCCAGCCCGGGAGATCCCTGAC CTGCTGGAAAAGGGGGAGCGGCTGCCCCAGCCCCCCATCTGCACCATTGATGTCT ACATGATCATGGTCAAATGTTGGATGATTGACTCTGAATGTCGGCCAAGATTCCGG GAGTTGGTGTCTGAATTCTCCCGCATGGCCAGGGACCCCCAGCGCTTTGTGGTCA TCCAGAATGAGGACTTGGGCCCAGCCAGTCCCTTGGACAGCACCTTCTACCGCTC ACTGCTGGAGGACGATGACATGGGGGACCTGGTGGATGCTGAGGAGTATCTGGTA CCCCAGCAGGGCTTCTTCTGTCCAGACCCTGCCCCGGGCGCTGGGGGCATGGTC CACCACAGGCACCGCAGCTCATCTACCAGGAGTGGCGGTGGGGACCTGACACTAG GGCTGGAGCCCTCTGAAGAGGAGGCCCCCAGGTCTCCACTGGCACCCTCCGAAG GGGCTGGCTCCGATGTATTTGATGGTGACCTGGGAATGGGGGCAGCCAAGGGGCT GCAAAGCCTCCCCACACATGACCCCAGCCCTCTACAGCGGTACAGTGAGGACCCC ACAGTACCCCTGCCCTCTGAGACTGATGGCTACGTTGCCCCCCTGACCTGCAGCC CCCAGCCTGAATATGTGAACCAGCCAGATGTTCGGCCCCAGCCCCCTTCGCCCCG AGAGGGCCCTCTGCCTGCTGCCCGACCTGCTGGTGCCACTCTGGAAAGGCCCAAG ACTCTCTCCCCAGGGAAGAATGGGGTCGTCAAAGACGTTTTTGCCTTTGGGGGTGC CGTGGAGAACCCCGAGTACTTGACACCCCAGGGAGGAGCTGCCCCTCAGCCCCAC CCTCCTCCTGCCTTCAGCCCAGCCTTCGACAACCTCTATTACTGGGACCAGGACCC ACCAGAGCGGGGGGCTCCACCCAGCACCTTCAAAGGGACACCTACGGCAGAGAA CCCAGAGTACCTGGGTCTGGACGTGCCAGTGTGAACCAGAAGGCCAAGTCCGCAG AAGCCCTGATGTGTCCTCAGGGAGCAGGGAAGGCCTGACTTCTGCTGGCATCAAG AGGTGGGAGGGCCCTCCGACCACTTCCAGGGGAACCTGCCATGCCAGGAACCTGT CCTAAGGAACCTTCCTTCCTGCTTGAGTTCCCAGATGGCTGGAAGGGGTCCAGCCT CGTTGGAAGAGGAACAGCACTGGGGAGTCTTTGTGGATTCTGAGGCCCTGCCCAA TGAGACTCTAGGGTCCAGTGGATGCCACAGCCCAGCTTGGCCCTTTCCTTCCAGAT CCTGGGTACTGAAAGCCTTAGGGAAGCTGGCCTGAGAGGGGAAGCGGCCCTAAG GGAGTGTCTAAGAACAAAAGCGACCCATTCAGAGACTGTCCCTGAAACCTAGTACT GCCCCCCATGAGGAAGGAACAGCAATGGTGTCAGTATCCAGGCTTTGTACAGAGT GCTTTTCTGTTTAGTTTTTACTTTTTTTGTTTTGTTTTTTTAAAGATGAAATAAAGACC CAGGGGGAGGACTACAAGGACGACGACGACAAGGGCCAGAGAGCGAGCGCGCAA CGGCGGCGACGGCGGCGACCCCACCGCGCATCCTGCCAGGCCTCCGGCGCCCA GCGCCCCACGCGCCCCCGCGCCCCCGCGCCCCCGCGCCCCTTTCTTCGCGCCCC CGCCCCTCGGCCCGCCAGGCCCCCTTGCCGGCCACCCGCCAGGCCCCGCGCCG GCCCGCCCGCCGCCCAGGACCGGCCCGCGCCCCGCAGGCCGCCCGCCGCCCGC GCCGCCATGGGAGTGGAGGGCTGCACCAAGTGCATCAAGTACCTGCTCTTCGTCT TCAATTTCGTCTTCTGGCTGGCTGGAGGCGTGATCCTGGGTGTGGCCCTGTGGCT CCGCCATGACCCGCAGACCACCAACCTCCTGTATCTGGAGCTGGGAGACAAGCCC GCGCCCAACACCTTCTATGTAGGCATCTACATCCTCATCGCTGTGGGCGCTGTCAT GATGTTCGTTGGCTTCCTGGGCTGCTACGGGGCCATCCAGGAATCCCAGTGCCTG CTGGGGACGTTCTTCACCTGCCTGGTCATCCTGTTTGCCTGTGAGGTGGCCGCCG GCATCTGGGGCTTTGTCAACAAGGACCAGATCGCCAAGGATGTGAAGCAGTTCTAT GACCAGGCCCTACAGCAGGCCGTGGTGGATGATGACGCCAACAACGCCAAGGCT GTGGTGAAGACCTTCCACGAGACGCTTGACTGCTGTGGCTCCAGCACACTGACTG CTTTGACCACCTCAGTGCTCAAGAACAATTTGTGTCCCTCGGGCAGCAACATCATC AGCAACCTCTTCAAGGAGGACTGCCACCAGAAGATCGATGACCTCTTCTCCGGGAA GCTGTACCTCATCGGCATTGCTGCCATCGTGGTCGCTGTGATCATGATCTTCGAGA TGATCCTGAGCATGGTGCTGTGCTGTGGCATCCGGAACAGCTCCGTGTACTGAGG CCCCGCAGCTCTGGCCACAGGGACCTCTGCAGTGCCCCCTAAGTGACCCGGACAC TTCCGAGGGGGCCATCACCGCCTGTGTATATAACGTTTCCGGTATTACTCTGCTAC ACGTAGCCTTTTTACTTTTGGGGTTTTGTTTTTGTTCTGAACTTTCCTGTTACCTTTTC AGGGCTGACGTCACATGTAGGTGGCGTGTATGAGTGGAGACGGGCCTGGGTCTTG GGGACTGGAGGGCAGGGGTCCTTCTGCCCTGGGGTCCCAGGGTGCTCTGCCTGC TCAGCCAGGCCTCTCCTGGGAGCCACTCGCCCAGAGACTCAGCTTGGCCAACTTG GGGGGCTGTGTCCACCCAGCCCGCCCGTCCTGTGGGCTGCACAGCTCACCTTGTT CCCTCCTGCCCCGGTTCGAGAGCCGAGTCTGTGGGCACTCTCTGCCTTCATGCAC CTGTCCTTTCTAACACGTCGCCTTCAACTGTAATCACAACATCCTGACTCCGTCATT TAATAAAGAAGGAACATCAGGCATGCTA (SEQ ID NO:44), or a nucleic acid sequence that hybridizes to a nucleic acid sequence consisting of SEQ ID NO:44 under stringent hybridization conditions.

In some embodiments, the fusion protein contains a Staphylococcus aureus antigen+MBP+CD9 and therefore can comprise the amino acid sequence: MKLFAFIFICVKSCSLLFMLNGNPRPEQLNKASEFTGLMDNMRYLYDDKHVSETNIKAQ EKFLQHDLLFKINGSKIDGSKILKTEFNNKSLSDKYKNKNVDLFGTNYYNQCYFSADNM ELNDGRLIEKTCMYGGVTEHDGNQIDKNNLTDNSHNILIKVYENERNTLSFDISTNKKNIT AQEIDYKVRNYLLKHKNLYKFNSSPYETGYIKFIEGNGHSFWYDMMPESGEKFYPTKYL LIYNDNKTVESKSINVEVHLTKKRSRRASWSGSTATRATTAWPRWARSSRRTPASRP WSTPTSWRRSSPRWPPPATAPTSSSGPTTGSAATPRAACWPRSPPTRPSRTSCTPS PGTPGTTASSPTPSPWRPASTTRTCCPTPPRPGRRSPPWTRSRPRARAPCSTCRSPT SPGPSPPTAATPSSTRTASTTSRTWAWTTPAPRPAPSWWTSEQAHERRHRLQHRRG RLQQGRDRHDHQRPLGLEQHRHQQGELRRDRAAHLQGPAQQALRGRAERRHQRRQ PQQGAGQGVPGELPADRRGPGGREQGQAPGRRGPEELRGGAGQGPQDRRHHGER PEGRDHAQHPPDERLLVRREDRRDQRRQRQADRGRGPEGRPDQQQQQQQQQQQQ QQPGHRGQGPAYSRLHLYPAPGPASPSCARPPVPHPFGPGVSPHHAGQRRHQVHQI PAVRILHLLACRDCCPCHWTMAPIRLSDQEHLRARNFQLLHRSLYSDRSRRPHDAGGL PGLLRGCAGVPVHAGTVLRLPLGDIRHNSCGHLGIFPQGGDGSPGVLQGHLQQAENQ GAPAGNAESHPLCVELLWFGWGRGTVYLRHLPQEGRTRNLHREVLSCHQRGLRQIPH HRRSGHRHCRGHDIWHDLQYDLVLCYPQEPRDGLESAYIPEQESLPMKIGGIFCLFVLF CLLFVVCFFATNFSIHSALLDKSSYFMFVFCFIQYHLLSGGFGLLWFIFFQLFVFACYIKQ KSCNERYYICTLDKILYIKEFFCLIDTNVYQLSSCNLYRQFDTKIMTMSKKKK (SEQ ID NO:45), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:45. Therefore, in some embodiments, the fusion protein is encoded by the nucleic acid sequence: ATGAAGTTATTTGCTTTTATCTTCATATGTGTTAAGTCTTGCAGCTTACTATTTATGTT AAATGGCAATCCTAGACCAGAACAATTGAATAAAGCGAGTGAATTCACTGGTCTAAT GGATAATATGAGGTATTTGTATGACGATAAACACGTATCAGAAACAAACATTAAAGC CCAAGAGAAGTTTTTACAACATGATTTATTATTTAAAATAAATGGCTCTAAAATTGAT GGTTCTAAAATTTTAAAAACAGAATTTAATAATAAAAGCCTTTCGGATAAATACAAAA ATAAAAACGTAGATTTGTTTGGGACAAACTATTATAATCAATGCTATTTTTCAGCGGA TAATATGGAATTAAATGATGGTAGACTAATTGAAAAAACGTGTATGTATGGCGGTGT GACCGAGCATGATGGAAATCAAATAGATAAAAATAATCTAACTGATAACTCTCATAA TATCTTAATTAAAGTATACGAAAACGAAAGAAACACATTATCTTTTGATATATCTACC AATAAGAAAAACATAACAGCACAAGAAATAGATTATAAAGTTAGAAACTATTTACTTA AGCATAAAAATTTATATAAATTTAATAGTTCGCCTTATGAGACTGGCTATATAAAGTT TATCGAAGGAAATGGTCATTCTTTTTGGTATGATATGATGCCTGAATCTGGTGAAAA ATTTTATCCGACTAAATATTTACTAATTTATAATGATAATAAGACAGTTGAGAGTAAAT CTATTAATGTAGAAGTTCATTTAACCAAAAAATAATGAAGATCGAGGAGGGCAAGCT GGTGATCTGGATCAACGGCGACAAGGGCTACAACGGCCTGGCCGAGGTGGGCAA GAAGTTCGAGAAGGACACCGGCATCAAGGTGACCGTGGAGCACCCCGACAAGCTG GAGGAGAAGTTCCCCCAGGTGGCCGCCACCGGCGACGGCCCCGACATCATCTTCT GGGCCCACGACAGGTTCGGCGGCTACGCCCAGAGCGGCCTGCTGGCCGAGATCA CCCCCGACAAGGCCTTCCAGGACAAGCTGTACCCCTTCACCTGGGACGCCGTGAG GTACAACGGCAAGCTGATCGCCTACCCCATCGCCGTGGAGGCCCTGAGCCTGATC TACAACAAGGACCTGCTGCCCAACCCCCCCAAGACCTGGGAGGAGATCCCCGCCC TGGACAAGGAGCTGAAGGCCAAGGGCAAGAGCGCCCTGATGTTCAACCTGCAGGA GCCCTACTTCACCTGGCCCCTGATCGCCGCCGACGGCGGCTACGCCTTCAAGTAC GAGAACGGCAAGTACGACATCAAGGACGTGGGCGTGGACAACGCCGGCGCCAAG GCCGGCCTGACCTTCCTGGTGGACCTGATCAGAACAAGCACATGAACGCCGACAC CGACTACAGCATCGCCGAGGCCGCCTTCAACAAGGGCGAGACCGCCATGACCATC AACGGCCCCTGGGCCTGGAGCAACATCGACACCAGCAAGGTGAACTACGGCGTGA CCGTGCTGCCCACCTTCAAGGGCCAGCCCAGCAAGCCCTTCGTGGGCGTGCTGAG CGCCGGCATCAACGCCGCCAGCCCCAACAAGGAGCTGGCCAAGGAGTTCCTGGA GAACTACCTGCTGACCGACGAGGGCCTGGAGGCCGTGAACAAGGACAAGCCCCT GGGCGCCGTGGCCCTGAAGAGCTACGAGGAGGAGCTGGCCAAGGACCCCAGGAT CGCCGCCACCATGGAGAACGCCCAGAAGGGCGAGATCATGCCCAACATCCCCCAG ATGAGCGCCTTCTGGTACGCCGTGAGGACCGCCGTGATCAACGCCGCCAGCGGC AGGCAGACCGTGGACGAGGCCCTGAAGGACGCCCAGACCAACAGCAGCAGCAAC AACAACAACAACAACAACAACAACAACCTGGGCATCGAGGGCAGGGACCAGCCTA CAGCCGCCTGCATCTGTATCCAGCGCCAGGTCCCGCCAGTCCCAGCTGCGCGCG CCCCCCAGTCCCGCACCCGTTCGGCCCAGGCTAAGTTAGCCCTCACCATGCCGGT CAAAGGAGGCACCAAGTGCATCAAATACCTGCTGTTCGGATTTAACTTCATCTTCTG GCTTGCCGGGATTGCTGTCCTTGCCATTGGACTATGGCTCCGATTCGACTCTCAGA CCAAGAGCATCTTCGAGCAAGAAACTAATAATAATAATTCCAGCTTCTACACAGGAG TCTATATTCTGATCGGAGCCGGCGCCCTCATGATGCTGGTGGGCTTCCTGGGCTG CTGCGGGGCTGTGCAGGAGTCCCAGTGCATGCTGGGACTGTTCTTCGGCTTCCTC TTGGTGATATTCGCCATTGAAATAGCTGCGGCCATCTGGGGATATTCCCACAAGGA TGAGGTGATTAAGGAAGTCCAGGAGTTTTACAAGGACACCTACAACAAGCTGAAAA CCAAGGATGAGCCCCAGCGGGAAACGCTGAAAGCCATCCACTATGCGTTGAACTG CTGTGGTTTGGCTGGGGGCGTGGAACAGTTTATCTCAGACATCTGCCCCAAGAAG GACGTACTCGAAACCTTCACCGTGAAGTCCTGTCCTGATGCCATCAAAGAGGTCTT CGACAATAAATTCCACATCATCGGCGCAGTGGGCATCGGCATTGCCGTGGTCATGA TATTTGGCATGATCTTCAGTATGATCTTGTGCTGTGCTATCCGCAGGAACCGCGAG ATGGTCTAGAGTCAGCTTACATCCCTGAGCAGGAAAGTTTACCCATGAAGATTGGT GGGATTTTTTGTTTGTTTGTTTTGTTTTGTTTGTTGTTTGTTGTTTGTTTTTTTGCCAC TAATTTTAGTATTCATTCTGCATTGCTAGATAAAAGCTGAAGTTACTTTATGTTTGTCT TTTAATGCTTCATTCAATATTGACATTTGTAGTTGAGCGGGGGGTTTGGTTTGCTTT GGTTTATATTTTTTCAGTTGTTTGTTTTTGCTTGTTATATTAAGCAGAAATCCTGCAAT GAAAGGTACTATATTTGCTAGACTCTAGACAAGATATTGTACATAAAAGAATTTTTTT GTCTTTAAATAGATACAAATGTCTATCAACTTTAATCAAGTTGTAACTTATATTGAAG ACAATTTGATACATAATAAAAAATTATGACAATGTCAAAAAAAAAAAAAAA (SEQ ID NO:46), or a nucleic acid sequence that hybridizes to a nucleic acid sequence consisting of SEQ ID NO:46 under stringent hybridization conditions.

In some embodiments, the fusion protein contains SARS-COV2 spike protein+Poly His tag+CD63+Myc tag+ICAM1 and therefore can comprise the amino acid sequence: MFVFLVLLPLVSSQCVNLTTRTQLPPAYTNSFTRGVYYPDKVFRSSVLHSTQDLFLPFF SNVTWFHAIHVSGTNGTKRFDNPVLPFNDGVYFASTEKSNIIRGWIFGTTLDSKTQSLLI VNNATNVVIKVCEFQFCNDPFLGVYYHKNNKSWMESEFRVYSSANNCTFEYVSQPFL MDLEGKQGNFKNLREFVFKNIDGYFKIYSKHTPINLVRDLPQGFSALEPLVDLPIGINITR FQTLLALHRSYLTPGDSSSGWTAGAAAYYVGYLQPRTFLLKYNENGTITDAVDCALDPL SETKCTLKSFTVEKGIYQTSNFRVQPTESIVRFPNITNLCPFGEVFNATRFASVYAWNRK RISNCVADYSVLYNSASFSTFKCYGVSPTKLNDLCFTNVYADSFVIRGDEVRQIAPGQT GKIADYNYKLPDDFTGCVIAWNSNNLDSKVGGNYNYLYRLFRKSNLKPFERDISTEIYQ AGSTPCNGVEGFNCYFPLQSYGFQPTNGVGYQPYRVVVLSFELLHAPATVCGPKKST NLVKNKCVNFNFNGLTGTGVLTESNKKFLPFQQFGRDIADTTDAVRDPQTLEILDITPCS FGGVSVITPGTNTSNQVAVLYQDVNCTEVPVAIHADQLTPTWRVYSTGSNVFQTRAGC LIGAEHVNNSYECDIPIGAGICASYQTQTNSPRRARSVASQSIIAYTMSLGAENSVAYSN NSIAIPTNFTISVTTEILPVSMTKTSVDCTMYICGDSTECSNLLLQYGSFCTQLNRALTGIA VEQDKNTQEVFAQVKQIYKTPPIKDFGGFNFSQILPDPSKPSKRSFIEDLLFNKVTLADA GFIKQYGDCLGDIAARDLICAQKFNGLTVLPPLLTDEMIAQYTSALLAGTITSGWTFGAG AALQIPFAMQMAYRFNGIGVTQNVLYENQKLIANQFNSAIGKIQDSLSSTASALGKLQDV VNQNAQALNTLVKQLSSNFGAISSVLNDILSRLDKVEAEVQIDRLITGRLQSLQTYVTQQ LIRAAEIRASANLAATKMSECVLGQSKRVDFCGKGYHLMSFPQSAPHGVVFLHVTYVP AQEKNFTTAPAICHDGKAHFPREGVFVSNGTHWFVTQRNFYEPQIITTDNTFVSGNCD VVIGIVNNTVYDPLQPELDSFKEELDKYFKNHTSPDVDLGDISGINASVVNIQKEIDRLNE VAKNLNESLIDLQELGKYEQYIKWPWYIWLGFIAGLIAIVMVTIMLCCMTSCCSCLKGCC SCGSCCKFDEDDSEPVLKGVKLHYTHHHHHHMAVEGGMKCVKFLLYVLLLAFCACAV GLIAVGVGAQLVLSQTIIQGATPGSLLPVVI AVGVFLFLVAFVGCCGACKENYCLMITFAI FLSLIMLVEVAAAIAGYVFRDKVMSEFNNNFRQQMENYPKNNHTASILDRMQADFKCC GAANYTDWEKIPSMSKNRVPDSCCINVTVGCGINFNEKAIHKEGCVEKIGGWLRKNVLV VAAAALGIAFVEVLGIVFACCLVKSIRSGYEVMEQKLISEEDLELLCYSELQPQPRYGSQ QPPARAARTPGPARGSVPRTWQCPDICVPLKSHPAPGRLRAGDMQHLLPAQVVGHR DPVAKGVAPAWEQPEGVTEQCARRPTNVLFKLPWAVNSNLPHRVLDSRTGGTGTPPL LAASGQEPYPTLPGGGWGTPGQPHRGAAPWGEGAETGASCGGARGHDHGAGEERS PWSQFLVPHTGPAAPRAGAVEHLGPLPAPDLCPASDSPTTCQPPGPRGGHAGDRGLF PGRAVPSLGGPGPPGTGGPEVEPHSHLWQRLLLGQGLSQCDRRGRGHPAADVCSNT GEPEPGDTADSDHLQLSGAQRDSDEARGLRRDRGDSEVGPPSQGDAEWGSSPATG PEGPAPAEGHPRGQRAQLLLLCNPGGGRPAYTQEPDPGASCPVWPPTGREGLSGKL DVARKFPADSNVPGLGEPIARAQVSKGWHFPTAHRGISDCHSRSGHLPLSGQEHSRG GHPQGDRECALPPVDCHHHCGSSRSHNGHCRPQHVPLPPAEDQEIQTTTGPKRDPH ETEHTSHASLNLSRDRASSSAFPYWWQWCHTEQSGRHMPCSYTYRPWDAGGQGIVL SQIQQHLGPWYLHTNTRPRISVVTLSQEEGARLKTLMDVKVPDERGSGGGDIAPPGHT TGKYNLLPIGYAEAPQTYRRSGPPTCVASKHKGPHFLTDASLGTAVYPQPLMICIYSFVI LPAIYVSFMAKTVSGLTELPVLITFKVTRYSCTGCTLQESAWQKDQMGLGLLIGQPAFP QKEFFYRHKSTIWTGNGYRFRDYPVRPYSSLPPKTDTFVSHLPTHIHFCQCSQHSAVM SGHECPGNMPKLCLVLLSCLHFTGSLHYAAPVSCSDQGPASSGEGGQGIGGLPPSFG SLIRVCVCVYVTSSRSVTQAGVQWCNHGSLQSPFGLKSSHLSLLSSWDHRLTTPHLAN LIFFFFPETGSRNIAQTSFVLVNKAFSTA (SEQ ID NO:47), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:47. Therefore, in some embodiments, the fusion protein is encoded by the nucleic acid sequence:

(SEQ ID NO:48)
ATGTTTGTTTTTCTTGTTTTATTGCCACTAGTCTCTAGTCAGTGTGTTAATCTTACAA
CCAGAACTCAATTACCCCCTGCATACACTAATTCTTTCACACGTGGTGTTTATTACC
CTGACAAAGTTTTCAGATCCTCAGTTTTACATTCAACTCAGGACTTGTTCTTACCTTT
CTTTTCCAATGTTACTTGGTTCCATGCTATACATGTCTCTGGGACCAATGGTACTAA
GAGGTTTGATAACCCTGTCCTACCATTTAATGATGGTGTTTATTTTGCTTCCACTGA
GAAGTCTAACATAATAAGAGGCTGGATTTTTGGTACTACTTTAGATTCGAAGACCCA
GTCCCTACTTATTGTTAATAACGCTACTAATGTTGTTATTAAAGTCTGTGAATTTCAA
TTTTGTAATGATCCATTTTTGGGTGTTTATTACCACAAAAACAACAAAAGTTGGATGG
AAAGTGAGTTCAGAGTTTATTCTAGTGCGAATAATTGCACTTTTGAATATGTCTCTCA
GCCTTTTCTTATGGACCTTGAAGGAAAACAGGGTAATTTCAAAAATCTTAGGGAATT
TGTGTTTAAGAATATTGATGGTTATTTTAAAATATATTCTAAGCACACGCCTATTAATT
TAGTGCGTGATCTCCCTCAGGGTTTTTCGGCTTTAGAACCATTGGTAGATTTGCCAA
TAGGTATTAACATCACTAGGTTTCAAACTTTACTTGCTTTACATAGAAGTTATTTGAC
TCCTGGTGATTCTTCTTCAGGTTGGACAGCTGGTGCTGCAGCTTATTATGTGGGTTA
TCTTCAACCTAGGACTTTTCTATTAAAATATAATGAAAATGGAACCATTACAGATGCT
GTAGACTGTGCACTTGACCCTCTCTCAGAAACAAAGTGTACGTTGAAATCCTTCACT
GTAGAAAAAGGAATCTATCAAACTTCTAACTTTAGAGTCCAACCAACAGAATCTATT
GTTAGATTTCCTAATATTACAAACTTGTGCCCTTTTGGTGAAGTTTTTAACGCCACCA
GATTTGCATCTGTTTATGCTTGGAACAGGAAGAGAATCAGCAACTGTGTTGCTGATT
ATTCTGTCCTATATAATTCCGCATCATTTTCCACTTTTAAGTGTTATGGAGTGTCTCC
TACTAAATTAAATGATCTCTGCTTTACTAATGTCTATGCAGATTCATTTGTAATTAGA
GGTGATGAAGTCAGACAAATCGCTCCAGGGCAAACTGGAAAGATTGCTGATTATAA
TTATAAATTACCAGATGATTTTACAGGCTGCGTTATAGCTTGGAATTCTAACAATCTT
GATTCTAAGGTTGGTGGTAATTATAATTACCTGTATAGATTGTTTAGGAAGTCTAATC
TCAAACCTTTTGAGAGAGATATTTCAACTGAAATCTATCAGGCCGGTAGCACACCTT
GTAATGGTGTTGAAGGTTTTAATTGTTACTTTCCTTTACAATCATATGGTTTCCAACC
CACTAATGGTGTTGGTTACCAACCATACAGAGTAGTAGTACTTTCTTTTGAACTTCTA
CATGCACCAGCAACTGTTTGTGGACCTAAAAAGTCTACTAATTTGGTTAAAAACAAA
TGTGTCAATTTCAACTTCAATGGTTTAACAGGCACAGGTGTTCTTACTGAGTCTAAC
AAAAAGTTTCTGCCTTTCCAACAATTTGGCAGAGACATTGCTGACACTACTGATGCT
GTCCGTGATCCACAGACACTTGAGATTCTTGACATTACACCATGTTCTTTTGGTGGT
GTCAGTGTTATAACACCAGGAACAAATACTTCTAACCAGGTTGCTGTTCTTTATCAG
GATGTTAACTGCACAGAAGTCCCTGTTGCTATTCATGCAGATCAACTTACTCCTACT
TGGCGTGTTTATTCTACAGGTTCTAATGTTTTTCAAACACGTGCAGGCTGTTTAATA
GGGGCTGAACATGTCAACAACTCATATGAGTGTGACATACCCATTGGTGCAGGTAT
ATGCGCTAGTTATCAGACTCAGACTAATTCTCCTCGGCGGGCACGTAGTGTAGCTA
GTCAATCCATCATTGCCTACACTATGTCACTTGGTGCAGAAAATTCAGTTGCTTACT
CTAATAACTCTATTGCCATACCCACAAATTTTACTATTAGTGTTACCACAGAAATTCT
ACCAGTGTCTATGACCAAGACATCAGTAGATTGTACAATGTACATTTGTGGTGATTC
AACTGAATGCAGCAATCTTTTGTTGCAATATGGCAGTTTTTGTACACAATTAAACCGT
GCTTTAACTGGAATAGCTGTTGAACAAGACAAAAACACCCAAGAAGTTTTTGCACAA
GTCAAACAAATTTACAAAACACCACCAATTAAAGATTTTGGTGGTTTTAATTTTTCAC
AAATATTACCAGATCCATCAAAACCAAGCAAGAGGTCATTTATTGAAGATCTACTTTT
CAACAAAGTGACACTTGCAGATGCTGGCTTCATCAAACAATATGGTGATTGCCTTG
GTGATATTGCTGCTAGAGACCTCATTTGTGCACAAAAGTTTAACGGCCTTACTGTTT
TGCCACCTTTGCTCACAGATGAAATGATTGCTCAATACACTTCTGCACTGTTAGCGG
GTACAATCACTTCTGGTTGGACCTTTGGTGCAGGTGCTGCATTACAAATACCATTTG
CTATGCAAATGGCTTATAGGTTTAATGGTATTGGAGTTACACAGAATGTTCTCTATG
AGAACCAAAAATTGATTGCCAACCAATTTAATAGTGCTATTGGCAAAATTCAAGACT
CACTTTCTTCCACAGCAAGTGCACTTGGAAAACTTCAAGATGTGGTCAACCAAAATG
CACAAGCTTTAAACACGCTTGTTAAACAACTTAGCTCCAATTTTGGTGCAATTTCAA
GTGTTTTAAATGATATCCTTTCACGTCTTGACAAAGTTGAGGCTGAAGTGCAAATTG
ATAGGTTGATCACAGGCAGACTTCAAAGTTTGCAGACATATGTGACTCAACAATTAA
TTAGAGCTGCAGAAATCAGAGCTTCTGCTAATCTTGCTGCTACTAAAATGTCAGAGT
GTGTACTTGGACAATCAAAAAGAGTTGATTTTTGTGGAAAGGGCTATCATCTTATGT
CCTTCCCTCAGTCAGCACCTCATGGTGTAGTCTTCTTGCATGTGACTTATGTCCCTG
CACAAGAAAAGAACTTCACAACTGCTCCTGCCATTTGTCATGATGGAAAAGCACACT
TTCCTCGTGAAGGTGTCTTTGTTTCAAATGGCACACACTGGTTTGTAACACAAAGGA
ATTTTTATGAACCACAAATCATTACTACAGACAACACATTTGTGTCTGGTAACTGTGA
TGTTGTAATAGGAATTGTCAACAACACAGTTTATGATCCTTTGCAACCTGAATTAGA
CTCATTCAAGGAGGAGTTAGATAAATATTTTAAGAATCATACATCACCAGATGTTGAT
TTAGGTGACATCTCTGGCATTAATGCTTCAGTTGTAAACATTCAAAAAGAAATTGAC
CGCCTCAATGAGGTTGCCAAGAATTTAAATGAATCTCTCATCGATCTCCAAGAACTT
GGAAAGTATGAGCAGTATATAAAATGGCCATGGTACATTTGGCTAGGTTTTATAGCT
GGCTTGATTGCCATAGTAATGGTGACAATTATGCTTTGCTGTATGACCAGTTGCTGT
AGTTGTCTCAAGGGCTGTTGTTCTTGTGGATCCTGCTGCAAATTTGATGAAGACGA
CTCTGAGCCAGTGCTCAAAGGAGTCAAATTACATTACACCCACCACCACCACCACC
ACATGGCGGTGGAAGGAGGAATGAAATGTGTGAAGTTCTTGCTCTACGTCCTCCTG
CTGGCCTTTTGCGCCTGTGCAGTGGGACTGATTGCCGTGGGTGTCGGGGCACAGC
TTGTCCTGAGTCAGACCATAATCCAGGGGGCTACCCCTGGCTCTCTGTTGCCAGTG
GTCATCATCGCAGTGGGTGTCTTCCTCTTCCTGGTGGCTTTTGTGGGCTGCTGCGG
GGCCTGCAAGGAGAACTATTGTCTTATGATCACGTTTGCCATCTTTCTGTCTCTTAT
CATGTTGGTGGAGGTGGCCGCAGCCATTGCTGGCTATGTGTTTAGAGATAAGGTGA
TGTCAGAGTTTAATAACAACTTCCGGCAGCAGATGGAGAATTACCCGAAAAACAAC
CACACTGCTTCGATCCTGGACAGGATGCAGGCAGATTTTAAGTGCTGTGGGGCTG
CTAACTACACAGATTGGGAGAAAATCCCTTCCATGTCGAAGAACCGAGTCCCCGAC
TCCTGCTGCATTAATGTTACTGTGGGCTGTGGGATTAATTTCAACGAGAAGGCGAT
CCATAAGGAGGGCTGTGTGGAGAAGATTGGGGGCTGGCTGAGGAAAAATGTGCTG
GTGGTAGCTGCAGCAGCCCTTGGAATTGCTTTTGTCGAGGTTTTGGGAATTGTCTT
TGCCTGCTGCCTCGTGAAGAGTATCAGAAGTGGCTACGAGGTGATGGAACAAAAAC
TCATCTCAGAAGAGGATCTGGAGCTCCTCTGCTACTCAGAGTTGCAACCTCAGCCT
CGCTATGGCTCCCAGCAGCCCCCGGCCCGCGCTGCCCGCACTCCTGGTCCTGCT
CGGGGCTCTGTTCCCAGGACCTGGCAATGCCCAGACATCTGTGTCCCCCTCAAAA
GTCATCCTGCCCCGGGGAGGCTCCGTGCTGGTGACATGCAGCACCTCCTGTGACC
AGCCCAAGTTGTTGGGCATAGAGACCCCGTTGCCTAAAAAGGAGTTGCTCCTGCCT
GGGAACAACCGGAAGGTGTATGAACTGAGCAATGTGCAAGAAGATAGCCAACCAAT
GTGCTATTCAAACTGCCCTGATGGGCAGTCAACAGCTAAAACCTTCCTCACCGTGT
ACTGGACTCCAGAACGGGTGGAACTGGCACCCCTCCCCTCTTGGCAGCCAGTGGG
CAAGAACCTTACCCTACGCTGCCAGGTGGAGGGTGGGGCACCCCGGGCCAACCT
CACCGTGGTGCTGCTCCGTGGGGAGAAGGAGCTGAAACGGGAGCCAGCTGTGGG
GGAGCCCGCTGAGGTCACGACCACGGTGCTGGTGAGGAGAGATCACCATGGAGC
CAATTTCTCGTGCCGCACTGAACTGGACCTGCGGCCCCAAGGGCTGGAGCTGTTT
GAGAACACCTCGGCCCCCTACCAGCTCCAGACCTTTGTCCTGCCAGCGACTCCCC
CACAACTTGTCAGCCCCCGGGTCCTAGAGGTGGACACGCAGGGGACCGTGGTCTG
TTCCCTGGACGGGCTGTTCCCAGTCTCGGAGGCCCAGGTCCACCTGGCACTGGGG
GACCAGAGGTTGAACCCCACAGTCACCTATGGCAACGACTCCTTCTCGGCCAAGG
CCTCAGTCAGTGTGACCGCAGAGGACGAGGGCACCCAGCGGCTGACGTGTGCAG
TAATACTGGGGAACCAGAGCCAGGAGACACTGCAGACAGTGACCATCTACAGCTTT
CCGGCGCCCAACGTGATTCTGACGAAGCCAGAGGTCTCAGAAGGGACCGAGGTGA
CAGTGAAGTGTGAGGCCCACCCTAGAGCCAAGGTGACGCTGAATGGGGTTCCAGC
CCAGCCACTGGGCCCGAGGGCCCAGCTCCTGCTGAAGGCCACCCCAGAGGACAA
CGGGCGCAGCTTCTCCTGCTCTGCAACCCTGGAGGTGGCCGGCCAGCTTATACAC
AAGAACCAGACCCGGGAGCTTCGTGTCCTGTATGGCCCCCGACTGGACGAGAGGG
ATTGTCCGGGAAACTGGACGTGGCCAGAAAATTCCCAGCAGACTCCAATGTGCCA
GGCTTGGGGGAACCCATTGCCCGAGCTCAAGTGTCTAAAGGATGGCACTTTCCCA
CTGCCCATCGGGGAATCAGTGACTGTCACTCGAGATCTTGAGGGCACCTACCTCTG
TCGGGCCAGGAGCACTCAAGGGGAGGTCACCCGCAAGGTGACCGTGAATGTGCT
CTCCCCCCGGTATGAGATTGTCATCATCACTGTGGTAGCAGCCGCAGTCATAATGG
GCACTGCAGGCCTCAGCACGTACCTCTATAACCGCCAGCGGAAGATCAAGAAATAC
AGACTACAACAGGCCCAAAAAGGGACCCCCATGAAACCGAACACACAAGCCACGC
CTCCCTGAACCTATCCCGGGACAGGGCCTCTTCCTCGGCCTTCCCATATTGGTGGC
AGTGGTGCCACACTGAACAGAGTGGAAGACATATGCCATGCAGCTACACCTACCG
GCCCTGGGACGCCGGAGGACAGGGCATTGTCCTCAGTCAGATACAACAGCATTTG
GGGCCATGGTACCTGCACACCTAAAACACTAGGCCACGCATCTGATCTGTAGTCAC
ATGACTAAGCCAAGAGGAAGGAGCAAGACTCAAGACATGATTGATGGATGTTAAAG
TCTAGCCTGATGAGAGGGGAAGTGGTGGGGGAGACATAGCCCCACCATGAGGACA
TACAACTGGGAAATACTGAAACTTGCTGCCTATTGGGTATGCTGAGGCCCCACAGA
CTTACAGAAGAAGTGGCCCTCCATAGACATGTGTAGCATCAAAACACAAAGGCCCA
CACTTCCTGACGGATGCCAGCTTGGGCACTGCTGTCTACTGACCCCAACCCTTGAT
GATATGTATTTATTCATTTGTTATTTTACCAGCTATTTATTGAGTGTCTTTTATGTAGG
CTAAATGAACATAGGTCTCTGGCCTCACGGAGCTCCCAGTCCTAATCACATTCAAG
GTCACCAGGTACAGTTGTACAGGTTGTACACTGCAGGAGAGTGCCTGGCAAAAAGA
TCAAATGGGGCTGGGACTTCTCATTGGCCAACCTGCCTTTCCCCAGAAGGAGTGAT
TTTTCTATCGGCACAAAAGCACTATATGGACTGGTAATGGTTACAGGTTCAGAGATT
ACCCAGTGAGGCCTTATTCCTCCCTTCCCCCCAAAACTGACACCTTTGTTAGCCAC
CTCCCCACCCACATACATTTCTGCCAGTGTTCACAATGACACTCAGCGGTCATGTCT
GGACATGAGTGCCCAGGGAATATGCCCAAGCTATGCCTTGTCCTCTTGTCCTGTTT
GCATTTCACTGGGAGCTTGCACTATGCAGCTCCAGTTTCCTGCAGTGATCAGGGTC
CTGCAAGCAGTGGGGAAGGGGGCCAAGGTATTGGAGGACTCCCTCCCAGCTTTGG
AAGCCTCATCCGCGTGTGTGTGTGTGTGTATGTGTAGACAAGCTCTCGCTCTGTCA
CCCAGGCTGGAGTGCAGTGGTGCAATCATGGTTCACTGCAGTCTTGACCTTTTGGG
CTCAAGTGATCCTCCCACCTCAGCCTCCTGAGTAGCTGGGACCATAGGCTCACAAC
ACCACACCTGGCAAATTTGATTTTTTTTTTTTTTCCAGAGACGGGGTCTCGCAACAT
TGCCCAGACTTCCTTTGTGTTAGTTAATAAAGCTTTCTCAACTGCC,

or a nucleic acid sequence that hybridizes to a nucleic acid sequence consisting of SEQ ID NO:48 under stringent hybridization conditions.

In some embodiments, the fusion protein contains Her-2/neu+HA+CD81+FLAG+ICAM4 and therefore can comprise the amino acid sequence: ISRAGPGSLARGPLVRAFCHGVSVPSVAAVFFGGRLPGSWERAGTPDAGAKPQGPSR ALRDLSALLGAHEPGVRGCAAGEVSAAQLQQQLSPAAEFQPPHPAAARQDAQRAGLG VLPAARREGLELPRALPRDLRRKNTLGHLQDHRLQCSRWATWWPGMEAGSSIPKAW SASPAWIWPTPPTSLLLDPATSGSPSATRASISTAWWSATARHPLHCSLGAPRPQLWP PVPSLPLGSSSLWALRTYASALSPRRKGGCSMPAEREKEEYETIWGNGHTWWLTPVIP ALWEAEAGESLEPRSSRPAWTTDPVYAKNTQISLVWWPAPVVPATREAELGGSFEPK SRLQALIVPLHSSLGDRARPCLQKNKNKNKYWRGNPLESIKASLT (SEQ ID NO:49), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:49. Therefore, in some embodiments, the fusion protein is encoded by the nucleic acid sequence: AAGGGGAGGTAACCCTGGCCCCTTTGGTCGGGGCCCCGGGCAGCCGCGCGCCCC TTCCCACGGGGCCCTTTACTGCGCCGCGCGCCCGGCCCCCACCCCTCGCAGCAC CCCGCGCCCCGCGCCCTCCCAGCCGGGTCCAGCCGGAGCCATGGGGCCGGAGC CGCAGTGAGCACCATGGAGCTGGCGGCCTTGTGCCGCTGGGGGCTCCTCCTCGC CCTCTTGCCCCCCGGAGCCGCGAGCACCCAAGTGTGCACCGGCACAGACATGAAG CTGCGGCTCCCTGCCAGTCCCGAGACCCACCTGGACATGCTCCGCCACCTCTACC AGGGCTGCCAGGTGGTGCAGGGAAACCTGGAACTCACCTACCTGCCCACCAATGC CAGCCTGTCCTTCCTGCAGGATATCCAGGAGGTGCAGGGCTACGTGCTCATCGCT CACAACCAAGTGAGGCAGGTCCCACTGCAGAGGCTGCGGATTGTGCGAGGCACCC AGCTCTTTGAGGACAACTATGCCCTGGCCGTGCTAGACAATGGAGACCCGCTGAA CAATACCACCCCTGTCACAGGGGCCTCCCCAGGAGGCCTGCGGGAGCTGCAGCTT CGAAGCCTCACAGAGATCTTGAAAGGAGGGGTCTTGATCCAGCGGAACCCCCAGC TCTGCTACCAGGACACGATTTTGTGGAAGGACATCTTCCACAAGAACAACCAGCTG GCTCTCACACTGATAGACACCAACCGCTCTCGGGCCTGCCACCCCTGTTCTCCGAT GTGTAAGGGCTCCCGCTGCTGGGGAGAGAGTTCTGAGGATTGTCAGAGCCTGACG CGCACTGTCTGTGCCGGTGGCTGTGCCCGCTGCAAGGGGCCACTGCCCACTGACT GCTGCCATGAGCAGTGTGCTGCCGGCTGCACGGGCCCCAAGCACTCTGACTGCCT GGCCTGCCTCCACTTCAACCACAGTGGCATCTGTGAGCTGCACTGCCCAGCCCTG GTCACCTACAACACAGACACGTTTGAGTCCATGCCCAATCCCGAGGGCCGGTATAC ATTCGGCGCCAGCTGTGTGACTGCCTGTCCCTACAACTACCTTTCTACGGACGTGG GATCCTGCACCCTCGTCTGCCCCCTGCACAACCAAGAGGTGACAGCAGAGGATGG AACACAGCGGTGTGAGAAGTGCAGCAAGCCCTGTGCCCGAGTGTGCTATGGTCTG GGCATGGAGCACTTGCGAGAGGTGAGGGCAGTTACCAGTGCCAATATCCAGGAGT TTGCTGGCTGCAAGAAGATCTTTGGGAGCCTGGCATTTCTGCCGGAGAGCTTTGAT GGGGACCCAGCCTCCAACACTGCCCCGCTCCAGCCAGAGCAGCTCCAAGTGTTTG AGACTCTGGAAGAGATCACAGGTTACCTATACATCTCAGCATGGCCGGACAGCCTG CCTGACCTCAGCGTCTTCCAGAACCTGCAAGTAATCCGGGGACGAATTCTGCACAA TGGCGCCTACTCGCTGACCCTGCAAGGGCTGGGCATCAGCTGGCTGGGGCTGCG CTCACTGAGGGAACTGGGCAGTGGACTGGCCCTCATCCACCATAACACCCACCTC TGCTTCGTGCACACGGTGCCCTGGGACCAGCTCTTTCGGAACCCGCACCAAGCTC TGCTCCACACTGCCAACCGGCCAGAGGACGAGTGTGTGGGCGAGGGCCTGGCCT GCCACCAGCTGTGCGCCCGAGGGCACTGCTGGGGTCCAGGGCCCACCCAGTGTG TCAACTGCAGCCAGTTCCTTCGGGGCCAGGAGTGCGTGGAGGAATGCCGAGTACT GCAGGGGCTCCCCAGGGAGTATGTGAATGCCAGGCACTGTTTGCCGTGCCACCCT GAGTGTCAGCCCCAGAATGGCTCAGTGACCTGTTTTGGACCGGAGGCTGACCAGT GTGTGGCCTGTGCCCACTATAAGGACCCTCCCTTCTGCGTGGCCCGCTGCCCCAG CGGTGTGAAACCTGACCTCTCCTACATGCCCATCTGGAAGTTTCCAGATGAGGAGG GCGCATGCCAGCCTTGCCCCATCAACTGCACCCACTCCTGTGTGGACCTGGATGA CAAGGGCTGCCCCGCCGAGCAGAGAGCCAGCCCTCTGACGTCCATCATCTCTGCG GTGGTTGGCATTCTGCTGGTCGTGGTCTTGGGGGTGGTCTTTGGGATCCTCATCAA GCGACGGCAGCAGAAGATCCGGAAGTACACGATGCGGAGACTGCTGCAGGAAAC GGAGCTGGTGGAGCCGCTGACACCTAGCGGAGCGATGCCCAACCAGGCGCAGAT GCGGATCCTGAAAGAGACGGAGCTGAGGAAGGTGAAGGTGCTTGGATCTGGCGCT TTTGGCACAGTCTACAAGGGCATCTGGATCCCTGATGGGGAGAATGTGAAAATTCC AGTGGCCATCAAAGTGTTGAGGGAAAACACATCCCCCAAAGCCAACAAAGAAATCT TAGACGAAGCATACGTGATGGCTGGTGTGGGCTCCCCATATGTCTCCCGCCTTCTG GGCATCTGCCTGACATCCACGGTGCAGCTGGTGACACAGCTTATGCCCTATGGCT GCCTCTTAGACCATGTCCGGGAAAACCGCGGACGCCTGGGCTCCCAGGACCTGCT GAACTGGTGTATGCAGATTGCCAAGGGGATGAGCTACCTGGAGGATGTGCGGCTC GTACACAGGGACTTGGCCGCTCGGAACGTGCTGGTCAAGAGTCCCAACCATGTCA AAATTACAGACTTCGGGCTGGCTCGGCTGCTGGACATTGACGAGACAGAGTACCAT GCAGATGGGGGCAAGGTGCCCATCAAGTGGATGGCGCTGGAGTCCATTCTCCGCC GGCGGTTCACCCACCAGAGTGATGTGTGGAGTTATGGTGTGACTGTGTGGGAGCT GATGACTTTTGGGGCCAAACCTTACGATGGGATCCCAGCCCGGGAGATCCCTGAC CTGCTGGAAAAGGGGGAGCGGCTGCCCCAGCCCCCCATCTGCACCATTGATGTCT ACATGATCATGGTCAAATGTTGGATGATTGACTCTGAATGTCGGCCAAGATTCCGG GAGTTGGTGTCTGAATTCTCCCGCATGGCCAGGGACCCCCAGCGCTTTGTGGTCA TCCAGAATGAGGACTTGGGCCCAGCCAGTCCCTTGGACAGCACCTTCTACCGCTC ACTGCTGGAGGACGATGACATGGGGGACCTGGTGGATGCTGAGGAGTATCTGGTA CCCCAGCAGGGCTTCTTCTGTCCAGACCCTGCCCCGGGCGCTGGGGGCATGGTC CACCACAGGCACCGCAGCTCATCTACCAGGAGTGGCGGTGGGGACCTGACACTAG GGCTGGAGCCCTCTGAAGAGGAGGCCCCCAGGTCTCCACTGGCACCCTCCGAAG GGGCTGGCTCCGATGTATTTGATGGTGACCTGGGAATGGGGGCAGCCAAGGGGCT GCAAAGCCTCCCCACACATGACCCCAGCCCTCTACAGCGGTACAGTGAGGACCCC ACAGTACCCCTGCCCTCTGAGACTGATGGCTACGTTGCCCCCCTGACCTGCAGCC CCCAGCCTGAATATGTGAACCAGCCAGATGTTCGGCCCCAGCCCCCTTCGCCCCG AGAGGGCCCTCTGCCTGCTGCCCGACCTGCTGGTGCCACTCTGGAAAGGCCCAAG ACTCTCTCCCCAGGGAAGAATGGGGTCGTCAAAGACGTTTTTGCCTTTGGGGGTGC CGTGGAGAACCCCGAGTACTTGACACCCCAGGGAGGAGCTGCCCCTCAGCCCCAC CCTCCTCCTGCCTTCAGCCCAGCCTTCGACAACCTCTATTACTGGGACCAGGACCC ACCAGAGCGGGGGGCTCCACCCAGCACCTTCAAAGGGACACCTACGGCAGAGAA CCCAGAGTACCTGGGTCTGGACGTGCCAGTGTGAACCAGAAGGCCAAGTCCGCAG AAGCCCTGATGTGTCCTCAGGGAGCAGGGAAGGCCTGACTTCTGCTGGCATCAAG AGGTGGGAGGGCCCTCCGACCACTTCCAGGGGAACCTGCCATGCCAGGAACCTGT CCTAAGGAACCTTCCTTCCTGCTTGAGTTCCCAGATGGCTGGAAGGGGTCCAGCCT CGTTGGAAGAGGAACAGCACTGGGGAGTCTTTGTGGATTCTGAGGCCCTGCCCAA TGAGACTCTAGGGTCCAGTGGATGCCACAGCCCAGCTTGGCCCTTTCCTTCCAGAT CCTGGGTACTGAAAGCCTTAGGGAAGCTGGCCTGAGAGGGGAAGCGGCCCTAAG GGAGTGTCTAAGAACAAAAGCGACCCATTCAGAGACTGTCCCTGAAACCTAGTACT GCCCCCCATGAGGAAGGAACAGCAATGGTGTCAGTATCCAGGCTTTGTACAGAGT GCTTTTCTGTTTAGTTTTTACTTTTTTTGTTTTGTTTTTTTAAAGATGAAATAAAGACC CAGGGGGAG (SEQ ID NO:50), or a nucleic acid sequence that hybridizes to a nucleic acid sequence consisting of SEQ ID NO:50 under stringent hybridization conditions.

In some embodiments, the fusion protein contains a Staphylococcus aureus antigen+MBP+CD9+Poly His+CD22 and therefore can comprise the amino acid sequence: MKLFAFIFICVKSCSLLFMLNGNPRPEQLNKASEFTGLMDNMRYLYDDKHVSETNIKAQ EKFLQHDLLFKINGSKIDGSKILKTEFNNKSLSDKYKNKNVDLFGTNYYNQCYFSADNM ELNDGRLIEKTCMYGGVTEHDGNQIDKNNLTDNSHNILIKVYENERNTLSFDISTNKKNIT AQEIDYKVRNYLLKHKNLYKFNSSPYETGYIKFIEGNGHSFWYDMMPESGEKFYPTKYL LIYNDNKTVESKSINVEVHLTKKRSRRASWSGSTATRATTAWPRWARSSRRTPASRP WSTPTSWRRSSPRWPPPATAPTSSSGPTTGSAATPRAACWPRSPPTRPSRTSCTPS PGTPGTTASSPTPSPWRPASTTRTCCPTPPRPGRRSPPWTRSRPRARAPCSTCRSPT SPGPSPPTAATPSSTRTASTTSRTWAWTTPAPRPAPSWWTSEQAHERRHRLQHRRG RLQQGRDRHDHQRPLGLEQHRHQQGELRRDRAAHLQGPAQQALRGRAERRHQRRQ PQQGAGQGVPGELPADRRGPGGREQGQAPGRRGPEELRGGAGQGPQDRRHHGER PEGRDHAQHPPDERLLVRREDRRDQRRQRQADRGRGPEGRPDQQQQQQQQQQQQ QQPGHRGQDQPTAACICIQRQVPPVPAARAPQSRTRSAQAKLALTMPVKGGTKCIKYL LFGFNFIFWLAGIAVLAIGLWLRFDSQTKSIFEQETNNNNSSFYTGVYILIGAGALMMLVG FLGCCGAVQESQCMLGLFFGFLLVIFAIEIAAAIWGYSHKDEVIKEVQEFYKDTYNKLKT KDEPQRETLKAIHYALNCCGLAGGVEQFISDICPKKDVLETFTVKSCPDAIKEVFDNKFHI IGAVGIGIAVVMIFGMIFSMILCCAIRRNREMVSQLTSLSRKVYPRLVGFFVCLFCFVCCL LFVFLPLILVFILHCIKAEVTLCLSFNASFNIDICSAGGLVCFGLYFFSCLFLLVILSRNPAM KGTIFARLTRYCTKNFFVFKIQMSINFNQVVTYIEDNLIHNKKLQCQKKKKHHHHHHTRK QACTQTRHHASPRPLAPAPGSRILGFLLKMGFAPNPLRLGGGLRLDPLHLQSPRWPGK LHPVPQSVQEHLEVWDKTLKHKGWEGSFAEKGAIPGRQEELHTEYPPGAPQQWSAG AEDGVQDEMDGTNTPQCLKAFSTSYPAPSRNSRVPGSHSDLLAEFLLLWVSDPIAVAP RGGSNEAGCCHLDLLDHQVCLHPERAQVLPTVESPWEDCDLPASGCRWEVPLQHGA AEREASSQEGDHSDSKPHADSRRRHSDPFLLQFQPQCYPVMETPWRLGGAIAWGAE DPKRWLGQHNHRLRSLLVLVGLPCRPECPVCPPRREGPENQAPFRDSLWKLGQPPM LLKQPPQRSPVLLGEKWQASGERKPAEFLHLPRRCWELQLLGEQLHRTDSVQGLDTS AVCTQEAACVHEPGGPSDGGEECNPDLERRQPSRLPLHLVLEPKPPLPQPEAEIGAGE GPALGCLLVPGDQQCGQGPFASQHPHRLLPGDHRQASGCGTRVLPRHPHPGNLWAQ APATLEEDTEPAGASGEFQRPELLCEEKGKGPPLRPPLPGMLQSNDGRWHLHHPALS RDEHTTNWRCRVLRDAETSPGLRHGHLFSIAQAPSGHYENVIPDFPEDEGIHYSELIQF GVGERPQAQENVDYVILKHHWMGCSRGTGGSGGQGSPRVFPR (SEQ ID NO:51), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:51. Therefore, in some embodiments, the fusion protein is encoded by the nucleic acid sequence: ATGAAGTTATTTGCTTTTATCTTCATATGTGTTAAGTCTTGCAGCTTACTATTTATGTT AAATGGCAATCCTAGACCAGAACAATTGAATAAAGCGAGTGAATTCACTGGTCTAAT GGATAATATGAGGTATTTGTATGACGATAAACACGTATCAGAAACAAACATTAAAGC CCAAGAGAAGTTTTTACAACATGATTTATTATTTAAAATAAATGGCTCTAAAATTGAT GGTTCTAAAATTTTAAAAACAGAATTTAATAATAAAAGCCTTTCGGATAAATACAAAA ATAAAAACGTAGATTTGTTTGGGACAAACTATTATAATCAATGCTATTTTTCAGCGGA TAATATGGAATTAAATGATGGTAGACTAATTGAAAAAACGTGTATGTATGGCGGTGT GACCGAGCATGATGGAAATCAAATAGATAAAAATAATCTAACTGATAACTCTCATAA TATCTTAATTAAAGTATACGAAAACGAAAGAAACACATTATCTTTTGATATATCTACC AATAAGAAAAACATAACAGCACAAGAAATAGATTATAAAGTTAGAAACTATTTACTTA AGCATAAAAATTTATATAAATTTAATAGTTCGCCTTATGAGACTGGCTATATAAAGTT TATCGAAGGAAATGGTCATTCTTTTTGGTATGATATGATGCCTGAATCTGGTGAAAA ATTTTATCCGACTAAATATTTACTAATTTATAATGATAATAAGACAGTTGAGAGTAAAT CTATTAATGTAGAAGTTCATTTAACCAAAAAATAATGAAGATCGAGGAGGGCAAGCT GGTGATCTGGATCAACGGCGACAAGGGCTACAACGGCCTGGCCGAGGTGGGCAA GAAGTTCGAGAAGGACACCGGCATCAAGGTGACCGTGGAGCACCCCGACAAGCTG GAGGAGAAGTTCCCCCAGGTGGCCGCCACCGGCGACGGCCCCGACATCATCTTCT GGGCCCACGACAGGTTCGGCGGCTACGCCCAGAGCGGCCTGCTGGCCGAGATCA CCCCCGACAAGGCCTTCCAGGACAAGCTGTACCCCTTCACCTGGGACGCCGTGAG GTACAACGGCAAGCTGATCGCCTACCCCATCGCCGTGGAGGCCCTGAGCCTGATC TACAACAAGGACCTGCTGCCCAACCCCCCCAAGACCTGGGAGGAGATCCCCGCCC TGGACAAGGAGCTGAAGGCCAAGGGCAAGAGCGCCCTGATGTTCAACCTGCAGGA GCCCTACTTCACCTGGCCCCTGATCGCCGCCGACGGCGGCTACGCCTTCAAGTAC GAGAACGGCAAGTACGACATCAAGGACGTGGGCGTGGACAACGCCGGCGCCAAG GCCGGCCTGACCTTCCTGGTGGACCTGATCAGAACAAGCACATGAACGCCGACAC CGACTACAGCATCGCCGAGGCCGCCTTCAACAAGGGCGAGACCGCCATGACCATC AACGGCCCCTGGGCCTGGAGCAACATCGACACCAGCAAGGTGAACTACGGCGTGA CCGTGCTGCCCACCTTCAAGGGCCAGCCCAGCAAGCCCTTCGTGGGCGTGCTGAG CGCCGGCATCAACGCCGCCAGCCCCAACAAGGAGCTGGCCAAGGAGTTCCTGGA GAACTACCTGCTGACCGACGAGGGCCTGGAGGCCGTGAACAAGGACAAGCCCCT GGGCGCCGTGGCCCTGAAGAGCTACGAGGAGGAGCTGGCCAAGGACCCCAGGAT CGCCGCCACCATGGAGAACGCCCAGAAGGGCGAGATCATGCCCAACATCCCCCAG ATGAGCGCCTTCTGGTACGCCGTGAGGACCGCCGTGATCAACGCCGCCAGCGGC AGGCAGACCGTGGACGAGGCCCTGAAGGACGCCCAGACCAACAGCAGCAGCAAC AACAACAACAACAACAACAACAACAACCTGGGCATCGAGGGCAGGACCAGCCTAC AGCCGCCTGCATCTGTATCCAGCGCCAGGTCCCGCCAGTCCCAGCTGCGCGCGC CCCCCAGTCCCGCACCCGTTCGGCCCAGGCTAAGTTAGCCCTCACCATGCCGGTC AAAGGAGGCACCAAGTGCATCAAATACCTGCTGTTCGGATTTAACTTCATCTTCTGG CTTGCCGGGATTGCTGTCCTTGCCATTGGACTATGGCTCCGATTCGACTCTCAGAC CAAGAGCATCTTCGAGCAAGAAACTAATAATAATAATTCCAGCTTCTACACAGGAGT CTATATTCTGATCGGAGCCGGCGCCCTCATGATGCTGGTGGGCTTCCTGGGCTGC TGCGGGGCTGTGCAGGAGTCCCAGTGCATGCTGGGACTGTTCTTCGGCTTCCTCT TGGTGATATTCGCCATTGAAATAGCTGCGGCCATCTGGGGATATTCCCACAAGGAT GAGGTGATTAAGGAAGTCCAGGAGTTTTACAAGGACACCTACAACAAGCTGAAAAC CAAGGATGAGCCCCAGCGGGAAACGCTGAAAGCCATCCACTATGCGTTGAACTGC TGTGGTTTGGCTGGGGGCGTGGAACAGTTTATCTCAGACATCTGCCCCAAGAAGG ACGTACTCGAAACCTTCACCGTGAAGTCCTGTCCTGATGCCATCAAAGAGGTCTTC GACAATAAATTCCACATCATCGGCGCAGTGGGCATCGGCATTGCCGTGGTCATGAT ATTTGGCATGATCTTCAGTATGATCTTGTGCTGTGCTATCCGCAGGAACCGCGAGA TGGTCTAGAGTCAGCTTACATCCCTGAGCAGGAAAGTTTACCCATGAAGATTGGTG GGATTTTTTGTTTGTTTGTTTTGTTTTGTTTGTTGTTTGTTGTTTGTTTTTTTGCCACT AATTTTAGTATTCATTCTGCATTGCTAGATAAAAGCTGAAGTTACTTTATGTTTGTCTT TTAATGCTTCATTCAATATTGACATTTGTAGTTGAGCGGGGGGTTTGGTTTGCTTTG GTTTATATTTTTTCAGTTGTTTGTTTTTGCTTGTTATATTAAGCAGAAATCCTGCAATG AAAGGTACTATATTTGCTAGACTCTAGACAAGATATTGTACATAAAAGAATTTTTTTG TCTTTAAATAGATACAAATGTCTATCAACTTTAATCAAGTTGTAACTTATATTGAAGAC AATTTGATACATAATAAAAAATTATGACAATGTCAAAAAAAAAAAAAACACCACCACC ACCACCACACGCGGAAACAGGCTTGCACCCAGACACGACACCATGCATCTCCTCG GCCCCTGGCTCCTGCTCCTGGTTCTAGAATACTTGGCTTTCTCTGACTCAAGTAAAT GGGTTTTTGAGCACCCTGAAACCCTCTACGCCTGGGAGGGGGCCTGCGTCTGGAT CCCCTGCACCTACAGAGCCCTAGATGGTGACCTGGAAAGCTTCATCCTGTTCCACA ATCCTGAGTATAACAAGAACACCTCGAAGTTTGATGGGACAAGACTCTATGAAAGC ACAAAGGATGGGAAGGTTCCTTCTGAGCAGAAAAGGGTGCAATTCCTGGGAGACA AGAATAAGAACTGCACACTGAGTATCCACCCGGTGCACCTCAATGACAGTGGTCAG CTGGGGCTGAGGATGGAGTCCAAGACTGAGAAATGGATGGAACGAATACACCTCA ATGTCTCTGAAAGGCCTTTTCCACCTCATATCCAGCTCCCTCCAGAAATTCAAGAGT CCCAGGAAGTCACTCTGACCTGCTTGCTGAATTTCTCCTGCTATGGGTATCCGATC CAATTGCAGTGGCTCCTAGAGGGGGTTCCAATGAGGCAGGCTGCTGTCACCTCGA CCTCCTTGACCATCAAGTCTGTCTTCACCCGGAGCGAGCTCAAGTTCTCCCCACAG TGGAGTCACCATGGGAAGATTGTGACCTGCCAGCTTCAGGATGCAGATGGGAAGT TCCTCTCCAATGACACGGTGCAGCTGAACGTGAAGCATCCTCCCAAGAAGGTGACC ACAGTGATTCAAAACCCCATGCCGATTCGAGAAGGAGACACAGTGACCCTTTCCTG TAACTACAATTCCAGTAACCCCAGTGTTACCCGGTATGAATGGAAACCCCATGGCG CCTGGGAGGAGCCATCGCTTGGGGTGCTGAAGATCCAAAACGTTGGCTGGGACAA CACAACCATCGCCTGCGCAGCTTGTAATAGTTGGTGCTCGTGGGCCTCCCCTGTC GCCCTGAATGTCCAGTATGCCCCCCGAGACGTGAGGGTCCGGAAAATCAAGCCCC TTTCCGAGATTCACTCTGGAAACTCGGTCAGCCTCCAATGTGACTTCTCAAGCAGC CACCCCAAAGAAGTCCAGTTCTTCTGGGAGAAAAATGGCAGGCTTCTGGGGAAAGA AAGCCAGCTGAATTTTGACTCCATCTCCCCAGAAGATGCTGGGAGTTACAGCTGCT GGGTGAACAACTCCATAGGACAGACAGCGTCCAAGGCCTGGACACTTGAAGTGCT GTATGCACCCAGGAGGCTGCGTGTGTCCATGAGCCCGGGGGACCAAGTGATGGA GGGGAAGAGTGCAACCCTGACCTGTGAGAGCGACGCCAACCCTCCCGTCTCCCAC TACACCTGGTTTGACTGGAATAACCAAAGCCTCCCCTACCACAGCCAGAAGCTGAG ATTGGAGCCGGTGAAGGTCCAGCACTCGGGTGCCTACTGGTGCCAGGGGACCAAC AGTGTGGGCAAGGGCCGTTCGCCTCTCAGCACCCTCACCGTCTACTATAGCCCGG AGACCATCGGCAGGCGAGTGGCTGTGGGACTCGGGTCCTGCCTCGCCATCCTCAT CCTGGCAATCTGTGGGCTCAAGCTCCAGCGACGTTGGAAGAGGACACAGAGCCAG CAGGGGCTTCAGGAGAATTCCAGCGGCCAGAGCTTCTTTGTGAGGAATAAAAAGGT TAGAAGGGCCCCCCTCTCTGAAGGCCCCCACTCCCTGGGATGCTACAATCCAATG ATGGAAGATGGCATTAGCTACACCACCCTGCGCTTTCCCGAGATGAACATACCACG AACTGGAGATGCAGAGTCCTCAGAGATGCAGAGACCTCCCCCGGACTGCGATGAC ACGGTCACTTATTCAGCATTGCACAAGCGCCAAGTGGGCACTATGAGAACGTCATT CCAGATTTTCCAGAAGATGAGGGGATTCATTACTCAGAGCTGATCCAGTTTGGGGT CGGGGAGCGGCCTCAGGCACAAGAAAATGTGGACTATGTGATCCTCAAACATTGA CACTGGATGGGCTGCAGCAGAGGCACTGGGGGCAGCGGGGGCCAGGGAAGTCC CCGAGTTTTCCCCAGAC (SEQ ID NO:52), or a nucleic acid sequence that hybridizes to a nucleic acid sequence consisting of SEQ ID NO:52 under stringent hybridization conditions.

In some embodiments, the fusion protein contains CD63+Poly His tag+ICAM1 and therefore can comprise the amino acid sequence: MAVEGGMKCVKFLLYVLLLAFCACAVGLIAVGVGAQLVLSQTIIQGATPGSLLPVVIIAVG VFLFLVAFVGCCGACKENYCLMITFAIFLSLIMLVEVAAAIAGYVFRDKVMSEFNNNFRQ QMENYPKNNHTASILDRMQADFKCCGAANYTDWEKIPSMSKNRVPDSCCINVTVGCGI NFNEKAIHKEGCVEKIGGWLRKNVLVVAAAALGIAFVEVLGIVFACCLVKSIRSGYEVMH HHHHHELLCYSELQPQPRYGSQQPPARAARTPGPARGSVPRTWQCPDICVPLKSHPA PGRLRAGDMQHLLPAQVVGHRDPVAKGVAPAWEQPEGVTEQCARRPTNVLFKLPWA VNSNLPHRVLDSRTGGTGTPPLLAASGQEPYPTLPGGGWGTPGQPHRGAAPWGEGA ETGASCGGARGHDHGAGEERSPWSQFLVPHTGPAAPRAGAVEHLGPLPAPDLCPAS DSPTTCQPPGPRGGHAGDRGLFPGRAVPSLGGPGPPGTGGPEVEPHSHLWQRLLLG QGLSQCDRRGRGHPAADVCSNTGEPEPGDTADSDHLQLSGAQRDSDEARGLRRDR GDSEVGPPSQGDAEWGSSPATGPEGPAPAEGHPRGQRAQLLLLCNPGGGRPAYTQE PDPGASCPVWPPTGREGLSGKLDVARKFPADSNVPGLGEPIARAQVSKGWHFPTAHR GISDCHSRSGHLPLSGQEHSRGGHPQGDRECALPPVDCHHHCGSSRSHNGHCRPQH VPLPPAEDQEIQTTTGPKRDPHETEHTSHASLNLSRDRASSSAFPYWWQWCHTEQSG RHMPCSYTYRPWDAGGQGIVLSQIQQHLGPWYLHTNTRPRISVVTLSQEEGARLKTLM DVKVPDERGSGGGDIAPPGHTTGKYNLLPIGYAEAPQTYRRSGPPTCVASKHKGPHFL TDASLGTAVYPQPLMICIYSFVILPAIYVSFMAKTVSGLTELPVLITFKVTRYSCTGCTLQE SAWQKDQMGLGLLIGQPAFPQKEFFYRHKSTIWTGNGYRFRDYPVRPYSSLPPKTDTF VSHLPTHIHFCQCSQHSAVMSGHECPGNMPKLCLVLLSCLHFTGSLHYAAPVSCSDQG PASSGEGGQGIGGLPPSFGSLIRVCVCVYVTSSRSVTQAGVQWCNHGSLQSPFGLKS SHLSLLSSWDHRLTTPHLANLIFFFFPETGSRNIAQTSFVLVNKAFSTA (SEQ ID NO:53), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:53. Therefore, in some embodiments, the fusion protein is encoded by the nucleic acid sequence: GAGCTCCTCTGCTACTCAGAGTTGCAACCTCAGCCTCGCTATGGCTCCCAGCAGCC CCCGGCCCGCGCTGCCCGCACTCCTGGTCCTGCTCGGGGCTCTGTTCCCAGGAC CTGGCAATGCCCAGACATCTGTGTCCCCCTCAAAAGTCATCCTGCCCCGGGGAGG CTCCGTGCTGGTGACATGCAGCACCTCCTGTGACCAGCCCAAGTTGTTGGGCATA GAGACCCCGTTGCCTAAAAAGGAGTTGCTCCTGCCTGGGAACAACCGGAAGGTGT ATGAACTGAGCAATGTGCAAGAAGATAGCCAACCAATGTGCTATTCAAACTGCCCT GATGGGCAGTCAACAGCTAAAACCTTCCTCACCGTGTACTGGACTCCAGAACGGGT GGAACTGGCACCCCTCCCCTCTTGGCAGCCAGTGGGCAAGAACCTTACCCTACGC TGCCAGGTGGAGGGTGGGGCACCCCGGGCCAACCTCACCGTGGTGCTGCTCCGT GGGGAGAAGGAGCTGAAACGGGAGCCAGCTGTGGGGGAGCCCGCTGAGGTCACG ACCACGGTGCTGGTGAGGAGAGATCACCATGGAGCCAATTTCTCGTGCCGCACTG AACTGGACCTGCGGCCCCAAGGGCTGGAGCTGTTTGAGAACACCTCGGCCCCCTA CCAGCTCCAGACCTTTGTCCTGCCAGCGACTCCCCCACAACTTGTCAGCCCCCGG GTCCTAGAGGTGGACACGCAGGGGACCGTGGTCTGTTCCCTGGACGGGCTGTTCC CAGTCTCGGAGGCCCAGGTCCACCTGGCACTGGGGGACCAGAGGTTGAACCCCA CAGTCACCTATGGCAACGACTCCTTCTCGGCCAAGGCCTCAGTCAGTGTGACCGC AGAGGACGAGGGCACCCAGCGGCTGACGTGTGCAGTAATACTGGGGAACCAGAG CCAGGAGACACTGCAGACAGTGACCATCTACAGCTTTCCGGCGCCCAACGTGATT CTGACGAAGCCAGAGGTCTCAGAAGGGACCGAGGTGACAGTGAAGTGTGAGGCC CACCCTAGAGCCAAGGTGACGCTGAATGGGGTTCCAGCCCAGCCACTGGGCCCGA GGGCCCAGCTCCTGCTGAAGGCCACCCCAGAGGACAACGGGCGCAGCTTCTCCT GCTCTGCAACCCTGGAGGTGGCCGGCCAGCTTATACACAAGAACCAGACCCGGGA GCTTCGTGTCCTGTATGGCCCCCGACTGGACGAGAGGGATTGTCCGGGAAACTGG ACGTGGCCAGAAAATTCCCAGCAGACTCCAATGTGCCAGGCTTGGGGGAACCCAT TGCCCGAGCTCAAGTGTCTAAAGGATGGCACTTTCCCACTGCCCATCGGGGAATCA GTGACTGTCACTCGAGATCTTGAGGGCACCTACCTCTGTCGGGCCAGGAGCACTC AAGGGGAGGTCACCCGCAAGGTGACCGTGAATGTGCTCTCCCCCCGGTATGAGAT TGTCATCATCACTGTGGTAGCAGCCGCAGTCATAATGGGCACTGCAGGCCTCAGCA CGTACCTCTATAACCGCCAGCGGAAGATCAAGAAATACAGACTACAACAGGCCCAA AAAGGGACCCCCATGAAACCGAACACACAAGCCACGCCTCCCTGAACCTATCCCG GGACAGGGCCTCTTCCTCGGCCTTCCCATATTGGTGGCAGTGGTGCCACACTGAA CAGAGTGGAAGACATATGCCATGCAGCTACACCTACCGGCCCTGGGACGCCGGAG GACAGGGCATTGTCCTCAGTCAGATACAACAGCATTTGGGGCCATGGTACCTGCAC ACCTAAAACACTAGGCCACGCATCTGATCTGTAGTCACATGACTAAGCCAAGAGGA AGGAGCAAGACTCAAGACATGATTGATGGATGTTAAAGTCTAGCCTGATGAGAGGG GAAGTGGTGGGGGAGACATAGCCCCACCATGAGGACATACAACTGGGAAATACTG AAACTTGCTGCCTATTGGGTATGCTGAGGCCCCACAGACTTACAGAAGAAGTGGCC CTCCATAGACATGTGTAGCATCAAAACACAAAGGCCCACACTTCCTGACGGATGCC AGCTTGGGCACTGCTGTCTACTGACCCCAACCCTTGATGATATGTATTTATTCATTT GTTATTTTACCAGCTATTTATTGAGTGTCTTTTATGTAGGCTAAATGAACATAGGTCT CTGGCCTCACGGAGCTCCCAGTCCTAATCACATTCAAGGTCACCAGGTACAGTTGT ACAGGTTGTACACTGCAGGAGAGTGCCTGGCAAAAAGATCAAATGGGGCTGGGAC TTCTCATTGGCCAACCTGCCTTTCCCCAGAAGGAGTGATTTTTCTATCGGCACAAAA GCACTATATGGACTGGTAATGGTTACAGGTTCAGAGATTACCCAGTGAGGCCTTAT TCCTCCCTTCCCCCCAAAACTGACACCTTTGTTAGCCACCTCCCCACCCACATACAT TTCTGCCAGTGTTCACAATGACACTCAGCGGTCATGTCTGGACATGAGTGCCCAGG GAATATGCCCAAGCTATGCCTTGTCCTCTTGTCCTGTTTGCATTTCACTGGGAGCTT GCACTATGCAGCTCCAGTTTCCTGCAGTGATCAGGGTCCTGCAAGCAGTGGGGAA GGGGGCCAAGGTATTGGAGGACTCCCTCCCAGCTTTGGAAGCCTCATCCGCGTGT GTGTGTGTGTGTATGTGTAGACAAGCTCTCGCTCTGTCACCCAGGCTGGAGTGCAG TGGTGCAATCATGGTTCACTGCAGTCTTGACCTTTTGGGCTCAAGTGATCCTCCCA CCTCAGCCTCCTGAGTAGCTGGGACCATAGGCTCACAACACCACACCTGGCAAATT TGATTTTTTTTTTTTTTCCAGAGACGGGGTCTCGCAACATTGCCCAGACTTCCTTTGT GTTAGTTAATAAAGCTTTCTCAACTGCC (SEQ ID NO:54), or a nucleic acid sequence that hybridizes to a nucleic acid sequence consisting of SEQ ID NO:54 under stringent hybridization conditions.

In some embodiments, the fusion protein can comprise the amino acid sequence: DQPTAACICIQRQVPPVPAARAPQSRTRSAQAKLALTMPVKGGTKCIKYLLFGFNFIFW LAGIAVLAIGLWLRFDSQTKSIFEQETNNNNSSFYTGVYILIGAGALMMLVGFLGCCGAV QESQCMLGLFFGFLLVIFAIEIAAAIWGYSHKDEVIKEVQEFYKDTYNKLKTKDEPQRETL KAIHYALNCCGLAGGVEQFISDICPKKDVLETFTVKSCPDAIKEVFDNKFHIIGAVGIGIAV VMIFGMIFSMILCCAIRRNREMVSQLTSLSRKVYPRLVGFFVCLFCFVCCLLFVFLPLILV FILHCIKAEVTLCLSFNASFNIDICSAGGLVCFGLYFFSCLFLLVILSRNPAMKGTIFARLT RYCTKNFFVFKIQMSINFNQVVTYIEDNLIHNKKLQCQKKKKRLQGRRRQGFSPMKIKHI LLLGVRHCMCPCLMLEMMHMKRLYMSNYPWVFISLRFSCCFIFPIRKRSKTVKSQITLA WYNLTAVLAIYMIISQGILAFSWMAHSAERKRTSVSQCMLPVKMKRKWTISTAELQYLN MRLSLFMGLTQLHLCMDQMMKMSLKRAWWRKTLSMLSTLAIVWLPMLVWKWYQILLA PKLISCSTFWMSRLLLENATLKIIKECVHSSKRVQCRPKASSSCPRLIRGYCTAKLIHIVIS CVILGKWKVEKKPVFISNWKAGHPFKWMRLQHSSLKEQQVFQSQIQELNTRMRMLRM FYWKDYIIKDPNVISPLFQVACYLDLLYFCSHMLCGRLASLKDNTNLSYKKKTEETVGVI STVKAMMIKDFFQIERMENRLRLRNLKDTVYKKKILFGLLLLMILHIMSSCKGKISAMITLD RRTAKVIIQPKIISQLLNGRNTKAFNLFKKSKPLKISNESITELNYTGEVLDLKYYLPYVLA SVKTPLGGQRFISNTSLILVQNMFFKNIIFRAVPKFSNEWTIITLKPFIYNTFPTGCVPTTIF FQQTMNIIVLAKLANFRLNMYTGLSLVKLLDNYFFIIMDFTIFLSVYIHVFLCRYIFTILPIYS SYNTPLSSIPRSNLQIFCYILKQKIVSVALYLIHADLENTETIPHLNQSCAKTRGPILHMYY VLCKILTITQLFPWMFFVTLYKYKCYLTWKRRNKIHKFKFINLAERYFNLYTVNIN ETTAK FSNVKYFYSLFILYEVLFMTFWILKNFFKYNHFCNIYFMLMIIIAEYHFILCLHKRAVAEFTS VIGSDQIRRQCGKTILGKISLYEVPATSQPSLMKVICSQACSDGEECSEICEGIVVKCKH KTSTQSVYTQETLHLCIFKKLCIVIKRLVILSGEHLGSMKNQAPINCLLQDCLGNLHLVLS YSELLRNNYIFQVIAQKQSTMVVQHRPLRYKGNTITYWARFCPVQNSICYQLTLLLVSIL KPYHFKKTGMGYSFLAGRLYNYVILKFNCSGLGSSESRQTYEICIIFVTEYRKFNIIDELK SKMKESKLFQNYLGILMYYDGCKVFCVSNKHIVKKRIIDIKKKK (SEQ ID NO:55), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:55. Therefore, in some embodiments, the fusion protein is encoded by the nucleic acid sequence: GTTTTTGAAGCCCCATGAAAATAAAACATATCTTGCTGTTGGGAGTATGAAGACATT GATGTTGAATGTGTCCTTGTTTAATGCTGGAGATGATGCATATGAAACGACTCTACA TGTCAAACTACCCGTGGGTCTTTATTTCATTAAGATTTTAGAGCTGTTGTTTCATTTT TCCCATTAGGAAGAGAAGCAAATAAACTGTGAAGTCACAGATAACTCTGGCGTGGT ACAACTTGACTGCAGTATTGGCTATATATATGTAGATCATCTCTCAAGGATAGATATT AGCTTTCTCCTGGATGTGAGCTCACTCAGCAGAGCGGAAGAGGACCTCAGTATCAC AGTGCATGCTACCTGTGAAAATGAAGAGGAAATGGACAATCTAAAGCACAGCAGAG TGACTGTAGCAATACCTTTAAAATATGAGGTTAAGCTGACTGTTCATGGGTTTGTAA ACCCAACTTCATTTGTGTATGGATCAAATGATGAAAATGAGCCTGAAACGTGCATGG TGGAGAAAATGAACTTAACTTTCCATGTTATCAACACTGGCAATAGTATGGCTCCCA ATGTTAGTGTGGAAATAATGGTACCAAATTCTTTTAGCCCCCAAACTGATAAGCTGT TCAACATTTTGGATGTCCAGACTACTACTGGAGAATGCCACTTTGAAAATTATCAAA GAGTGTGTGCATTAGAGCAGCAAAAGAGTGCAATGCAGACCTTGAAAGGCATAGTC CAGTTCTTGTCCAAGACTGATAAGAGGCTATTGTACTGCATAAAAGCTGATCCACAT TGTTTAAATTTCTTGTGTAATTTTGGGAAAATGGAAAGTGGAAAAGAAGCCAGTGTT CATATCCAACTGGAAGGCCGGCCATCCATTTTAGAAATGGATGAGACTTCAGCACT CAAGTTTGAAATAAGAGCAACAGGTTTTCCAGAGCCAAATCCAAGAGTAATTGAACT AAACAAGGATGAGAATGTTGCGCATGTTCTACTGGAAGGACTACATCATCAAAGAC CCAAACGTTATTTCACCATAGTGATTATTTCAAGTAGCTTGCTACTTGGACTTATTGT ACTTCTGTTGATCTCATATGTTATGTGGAAGGCTGGCTTCTTTAAAAGACAATACAA ATCTATCCTACAAGAAGAAAACAGAAGAGACAGTTGGAGTTATATCAACAGTAAAAG CAATGATGATTAAGGACTTCTTTCAAATTGAGAGAATGGAAAACAGACTCAGGTTGT AGTAAAGAAATTTAAAAGACACTGTTTACAAGAAAAAATGAATTTTGTTTGGACTTCT TTTACTCATGATCTTGTGACATATTATGTCTTCATGCAAGGGGAAAATCTCAGCAAT GATTACTCTTTGAGATAGAAGAACTGCAAAGGTAATAATACAGCCAAAGATAATCTC TCAGCTTTTAAATGGGTAGAGAAACACTAAAGCATTCAATTTATTCAAGAAAAGTAA GCCCTTGAAGATATCTTGAAATGAAAGTATAACTGAGTTAAATTATACTGGAGAAGT CTTAGACTTGAAATACTACTTACCATATGTGCTTGCCTCAGTAAAATGAACCCCACT GGGTGGGCAGAGGTTCATTTCAAATACATCTTTGATACTTGTTCAAAATATGTTCTTT AAAAATATAATTTTTTAGAGAGCTGTTCCCAAATTTTCTAACGAGTGGACCATTATCA CTTTAAAGCCCTTTATTTATAATACATTTCCTACGGGCTGTGTTCCAACAACCATTTT TTTTCAGCAGACTATGAATATTATAGTATTATAGGCCAAACTGGCAAACTTCAGACT GAACATGTACACTGGTTTGAGCTTAGTGAAATGACTTCTGGATAATTATTTTTTTATA ATTATGGATTTCACCATCTTTCTTTCTGTATATATACATGTGTTTTTATGTAGGTATAT ATTTACCATTCTTCCTATCTATTCTTCCTATAACACACCTTTATCAAGCATACCCAGG AGTAATCTTCAAATCTTTTGTTATATTCTGAAACAAAAGATTGTGAGTGTTGCACTTT ACCTGATACATGCTGATTTAGAAAATACAGAAACCATACCTCACTAATAACTTTAAAA TCAAAGCTGTGCAAAGACTAGGGGGCCTATACTTCATATGTATTATGTACTATGTAA AATATTGACTATCACACAACTATTTCCTTGGATGTAATTCTTTGTTACCCTTTACAAG TATAAGTGTTACCTTACATGGAAACGAAGAAACAAAATTCATAAATTTAAATTCATAA ATTTAGCTGAAAGATACTGATTCAATTTGTATACAGTGAATATAAATGAGACGACAG CAAAATTTTCATGAAATGTAAAATATTTTTATAGTTTGTTCATACTATATGAGGTTCTA TTTTAAATGACTTTCTGGATTTTAAAAAATTTCTTTAAATACAATCATTTTTGTAATATT TATTTTATGCTTATGATCTAGATAATTGCAGAATATCATTTTATCTGACTCTGTCTTCA TAAGAGAGCTGTGGCCGAATTTTGAACATCTGTTATAGGGAGTGATCAAATTAGAAG GCAATGTGGAAAAACAATTCTGGGAAAGATTTCTTTATATGAAGTCCCTGCCACTAG CCAGCCATCCTAATTGATGAAAGTTATCTGTTCACAGGCCTGCAGTGATGGTGAGG AATGTTCTGAGATTTGCGAAGGCATTTGAGTAGTGAAATGTAAGCACAAAACCTCCT GAACCCAGAGTGTGTATACACAGGAATAAACTTTATGACATTTATGTATTTTTAAAAA ACTTTGTATCGTTATAAAAAGGCTAGTCATTCTTTCAGGAGAACATCTAGGATCATA GATGAAAAATCAAGCCCCGATTTAGAACTGTCTTCTCCAGGATTGTCTCTAAGGAAA TTTACATTTGGTTCTTTCCTACTCAGAACTACTCAGAAACAACTATATATTTCAGGTT ATTTGAGCACAGTGAAAGCAGAGTACTATGGTTGTCCAACACAGGCCTCTCAGATA CAAGGGGAACACAATTACATATTGGGCTAGATTTTGCCCAGTTCAAAATAGTATTTG TTATCAACTTACTTTGTTACTTGTATCATGAATTTTAAAACCCTACCACTTTAAGAAGA CAGGGATGGGTTATTCTTTTTTGGCAGGTAGGCTATATAACTATGTGATTTTGAAAT TTAACTGCTCTGGATTAGGGAGCAGTGAATCAAGGCAGACTTATGAAATCTGTATTA TATTTGTAACAGAATATAGGAAATTTAACATAATTGATGAGCTCAAATCCTGAAAAAT GAAAGAATCCAAATTATTTCAGAATTATCTAGGTTAAATATTGATGTATTATGATGGT TGCAAAGTTTTTTGTGTGTCCAATAAACACATTGTAAAAAAAAGAATTTGAATTGATA TTTAAAAAAAAAAAAAAA (SEQ ID NO:56), or a nucleic acid sequence that hybridizes to a nucleic acid sequence consisting of SEQ ID NO:56 under stringent hybridization conditions.

In some embodiments, the fusion protein contains CD81+Myc Tag+KRT14 and therefore can comprise the amino acid sequence: GQRASAQRRRRRRPHRASCQASGAQRPTRPRAPAPPRPFLRAPAPRPARPPCRPPA RPRAGPPAAQDRPAPRRPPAARAAMGVEGCTKCIKYLLFVFNFVFWLAGGVILGVALW LRHDPQTTNLLYLELGDKPAPNTFYVGIYILIAVGAVMMFVGFLGCYGAIQESQCLLGTF FTCLVILFACEVAAGIWGFVNKDQIAKDVKQFYDQALQQAVVDDDANNAKAVVKTFHET LDCCGSSTLTALTTSVLKNNLCPSGSNIISNLFKEDCHQKIDDLFSGKLYLIGIAAIVVAVI MIFEMILSMVLCCGIRNSSVYGPAALATGTSAVPPKPGHFRGGHHRLCIRFRYYSATRS LFTFGVLFLFTFLLPFQGRHMVACMSGDGPGSWGLEGRGPSALGSQGALPAQPGLS WEPLAQRLSLANLGGCVHPARPSCGLHSSPCSLLPRFESRVCGHSLPSCTCPFHVAF NCNHNILTPSFNKEGTSGMLEQKLISEEDLTRAPSLHSANCSLAHLPPLHHDHLQPPVH LLQLHEGLLRHRGRHRGRLQPHLLRPGRRVLPRPQHLRGRPVCLILPLLLWGSLRAGG RLWRWLQQQQQQLWWLWGRIWWWPWCWLGWWLWWWLCWWWASGGQEGDHA EPQPPGLLPGQGACSGGGQRRPGSEDPLVPEAAACDQRLQSLLQDHGPEEQDSHSH SGQCQCPSADQCPSGRGLPHQVDRVEPAHECGSRHQWPAQGAGRTDPGQSPGDA DEPEGGAGLPEEEPRGGDECPERPGGWRCQCGDGRCTWRGPEPHSERDAPVEDG REEPQGCRGMVLHQDRGAEPRGGHQQRAGAERQERDLGAPAHHAEPGDAAVPAQH ESIPGEQPGGDQRSLLHAAGPDPGDDWQRGGAAGPAPLRDGAAEPGVQDPAGREDA AGAGDRHLPPPAGGRGRPPLLLPVLLWIAVIQRCDLLQPPNPHQGHGCARWQGGVHP RAGPSHQELRLPSPAQAEAPRVDTDPTGRSPLLPKHFTAGPCFTLTPSWQSIQLHYLS CI (SEQ ID NO:57), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:57. Therefore, in some embodiments, the fusion protein is encoded by the nucleic acid sequence: GGCCAGAGAGCGAGCGCGCAACGGCGGCGACGGCGGCGACCCCACCGCGCATC CTGCCAGGCCTCCGGCGCCCAGCGCCCCACGCGCCCCCGCGCCCCCGCGCCCC CGCGCCCCTTTCTTCGCGCCCCCGCCCCTCGGCCCGCCAGGCCCCCTTGCCGGC CACCCGCCAGGCCCCGCGCCGGCCCGCCCGCCGCCCAGGACCGGCCCGCGCCC CGCAGGCCGCCCGCCGCCCGCGCCGCCATGGGAGTGGAGGGCTGCACCAAGTG CATCAAGTACCTGCTCTTCGTCTTCAATTTCGTCTTCTGGCTGGCTGGAGGCGTGA TCCTGGGTGTGGCCCTGTGGCTCCGCCATGACCCGCAGACCACCAACCTCCTGTA TCTGGAGCTGGGAGACAAGCCCGCGCCCAACACCTTCTATGTAGGCATCTACATCC TCATCGCTGTGGGCGCTGTCATGATGTTCGTTGGCTTCCTGGGCTGCTACGGGGC CATCCAGGAATCCCAGTGCCTGCTGGGGACGTTCTTCACCTGCCTGGTCATCCTGT TTGCCTGTGAGGTGGCCGCCGGCATCTGGGGCTTTGTCAACAAGGACCAGATCGC CAAGGATGTGAAGCAGTTCTATGACCAGGCCCTACAGCAGGCCGTGGTGGATGAT GACGCCAACAACGCCAAGGCTGTGGTGAAGACCTTCCACGAGACGCTTGACTGCT GTGGCTCCAGCACACTGACTGCTTTGACCACCTCAGTGCTCAAGAACAATTTGTGT CCCTCGGGCAGCAACATCATCAGCAACCTCTTCAAGGAGGACTGCCACCAGAAGA TCGATGACCTCTTCTCCGGGAAGCTGTACCTCATCGGCATTGCTGCCATCGTGGTC GCTGTGATCATGATCTTCGAGATGATCCTGAGCATGGTGCTGTGCTGTGGCATCCG GAACAGCTCCGTGTACTGAGGCCCCGCAGCTCTGGCCACAGGGACCTCTGCAGTG CCCCCTAAGTGACCCGGACACTTCCGAGGGGGCCATCACCGCCTGTGTATATAAC GTTTCCGGTATTACTCTGCTACACGTAGCCTTTTTACTTTTGGGGTTTTGTTTTTGTT CTGAACTTTCCTGTTACCTTTTCAGGGCTGACGTCACATGTAGGTGGCGTGTATGA GTGGAGACGGGCCTGGGTCTTGGGGACTGGAGGGCAGGGGTCCTTCTGCCCTGG GGTCCCAGGGTGCTCTGCCTGCTCAGCCAGGCCTCTCCTGGGAGCCACTCGCCCA GAGACTCAGCTTGGCCAACTTGGGGGGCTGTGTCCACCCAGCCCGCCCGTCCTGT GGGCTGCACAGCTCACCTTGTTCCCTCCTGCCCCGGTTCGAGAGCCGAGTCTGTG GGCACTCTCTGCCTTCATGCACCTGTCCTTTCTAACACGTCGCCTTCAACTGTAATC ACAACATCCTGACTCCGTCATTTAATAAAGAAGGAACATCAGGCATGCTAGAACAAA AACTCATCTCAGAAGAGGATCTGACCCGAGCACCTTCTCTTCACTCAGCCAACTGC TCGCTCGCTCACCTCCCTCCTCTGCACCATGACCACCTGCAGCCGCCAGTTCACCT CCTCCAGCTCCATGAAGGGCTCCTGCGGCATCGGGGGCGGCATCGGGGGCGGCT CCAGCCGCATCTCCTCCGTCCTGGCCGGAGGGTCCTGCCGCGCCCCCAGCACCT ACGGGGGCGGCCTGTCTGTCTCATCCTCCCGCTTCTCCTCTGGGGGAGCCTGCGG GCTGGGGGGCGGCTATGGCGGTGGCTTCAGCAGCAGCAGCAGCAGCTTTGGTAG TGGCTTTGGGGGAGGATATGGTGGTGGCCTTGGTGCTGGCTTGGGTGGTGGCTTT GGTGGTGGCTTTGCTGGTGGTGATGGGCTTCTGGTGGGCAGTGAGAAGGTGACCA TGCAGAACCTCAATGACCGCCTGGCCTCCTACCTGGACAAGGTGCGTGCTCTGGA GGAGGCCAACGCCGACCTGGAAGTGAAGATCCGTGACTGGTACCAGAGGCAGCG GCCTGCTGAGATCAAAGACTACAGTCCCTACTTCAAGACCATTGAGGACCTGAGGA ACAAGATTCTCACAGCCACAGTGGACAATGCCAATGTCCTTCTGCAGATTGACAAT GCCCGTCTGGCCGCGGATGACTTCCGCACCAAGTATGAGACAGAGTTGAACCTGC GCATGAGTGTGGAAGCCGACATCAATGGCCTGCGCAGGGTGCTGGACGAACTGAC CCTGGCCAGAGCTGACCTGGAGATGCAGATTGAGAGCCTGAAGGAGGAGCTGGC CTACCTGAAGAAGAACCACGAGGAGGAGATGAATGCCCTGAGAGGCCAGGTGGGT GGAGATGTCAATGTGGAGATGGACGCTGCACCTGGCGTGGACCTGAGCCGCATTC TGAACGAGATGCGTGACCAGTATGAGAAGATGGCAGAGAAGAACCGCAAGGATGC CGAGGAATGGTTCTTCACCAAGACAGAGGAGCTGAACCGCGAGGTGGCCACCAAC AGCGAGCTGGTGCAGAGCGGCAAGAGCGAGATCTCGGAGCTCCGGCGCACCATG CAGAACCTGGAGATTGAGCTGCAGTCCCAGCTCAGCATGAAAGCATCCCTGGAGA ACAGCCTGGAGGAGACCAAAGGTCGCTACTGCATGCAGCTGGCCCAGATCCAGGA GATGATTGGCAGCGTGGAGGAGCAGCTGGCCCAGCTCCGCTGCGAGATGGAGCA GCAGAACCAGGAGTACAAGATCCTGCTGGACGTGAAGACGCGGCTGGAGCAGGA GATCGCCACCTACCGCCGCCTGCTGGAGGGCGAGGACGCCCACCTCTCCTCCTCC CAGTTCTCCTCTGGATCGCAGTCATCCAGAGATGTGACCTCCTCCAGCCGCCAAAT CCGCACCAAGGTCATGGATGTGCACGATGGCAAGGTGGTGTCCACCCACGAGCAG GTCCTTCGCACCAAGAACTGAGGCTGCCCAGCCCCGCTCAGGCCTAGGAGGCCCC CCGTGTGGACACAGATCCCACTGGAAGATCCCCTCTCCTGCCCAAGCACTTCACAG CTGGACCCTGCTTCACCCTCACCCCCTCCTGGCAATCAATACAGCTTCATTATCTGA GTTGCATAA (SEQ ID NO:58), or a nucleic acid sequence that hybridizes to a nucleic acid sequence consisting of SEQ ID NO:58 under stringent hybridization conditions.

In order to express a polypeptide or functional nucleic acid, the disclosed polynucleotides may be inserted into appropriate expression vector. Therefore, also disclosed is a non-viral vector comprising a polynucleotide disclosed herein, wherein the nucleic acid sequences are operably linked to an expression control sequence. In some embodiments, the nucleic acid sequences are operably linked to a single expression control sequence. In other embodiments, the nucleic acid sequences are operably linked to two or more separate expression control sequences.

Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al., Molecular Cloning, A Laboratory Manual (Cold Spring Harbor Press, Plainview, N.Y., 1989), and Ausubel et al., Current Protocols in Molecular Biology (John Wiley & Sons, New York, N.Y., 1989).

Expression vectors generally contain regulatory sequences necessary elements for the translation and/or transcription of the inserted coding sequence. For example, the coding sequence is preferably operably linked to a promoter and/or enhancer to help control the expression of the desired gene product.

The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Promoters used in biotechnology are of different types according to the intended type of control of gene expression. They can be generally divided into constitutive promoters, tissue-specific or development-stage-specific promoters, inducible promoters, and synthetic promoters.

Constitutive promoters direct expression in virtually all tissues and are largely, if not entirely, independent of environmental and developmental factors. As their expression is normally not conditioned by endogenous factors, constitutive promoters are usually active across species and even across kingdoms. Examples of constitutive promoters include CMV, EF1a, SV40, PGK1, Ubc, Human beta actin, and CAG.

The performance of inducible promoters is not conditioned to endogenous factors but to environmental conditions and external stimuli that can be artificially controlled. Within this group, there are promoters modulated by abiotic factors such as light, oxygen levels, heat, cold and wounding. Since some of these factors are difficult to control outside an experimental setting, promoters that respond to chemical compounds, not found naturally in the organism of interest, are of particular interest. Along those lines, promoters that respond to antibiotics, copper, alcohol, steroids, and herbicides, among other compounds, have been adapted and refined to allow the induction of gene activity at will and independently of other biotic or abiotic factors.

Disclosed are non-viral vectors containing one or more polynucleotides disclosed herein operably linked to an expression control sequence. Examples of such non-viral vectors include the oligonucleotide alone or in combination with a suitable protein, polysaccharide or lipid formulation. Non-viral methods present certain advantages over viral methods, with simple large scale production and low host immunogenicity being just two. Previously, low levels of transfection and expression of the gene held non-viral methods at a disadvantage; however, recent advances in vector technology have yielded molecules and techniques with transfection efficiencies similar to those of viruses.

Examples of suitable non-viral vectors include, but are not limited to pIRES-hrGFP-2a, pCMV6, pMAX, pCAG, pAd-IRES-GFP, and pCDNA3.0.

The compositions disclosed can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Methods

Also disclosed herein is a method of vaccinating a subject that involves transfecting skin cells of the subject with a vaccine composition disclosed herein containing the first and second polynucleotides. As disclosed herein, this method will cause skin-resident skin cells to produce EVs containing the viral antigen and decorated on the surface with an APC-targeting ligand.

In some embodiments, the polynucleotides are delivered to skin cells intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some embodiments, a viral vector can be used. However, in other embodiments, the polynucleotides are not delivered virally.

Electroporation is a technique in which an electrical field is applied to cells in order to increase permeability of the cell membrane, allowing cargo (e.g., reprogramming factors) to be introduced into cells. Electroporation is a common technique for introducing foreign DNA into cells.

Tissue nanotransfection allows for direct cytosolic delivery of cargo (e.g., reprogramming factors) into cells by applying a highly intense and focused electric field through arrayed nanochannels, which benignly nanoporates the juxtaposing tissue cell members, and electrophoretically drives cargo into the cells.

In some embodiments, the disclosed compositions are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of the disclosed compositions administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

### Example Embodiments

Embodiment 1. A vaccine composition, comprising
(a) a first polynucleotide encoding or comprising a viral, bacterial, or tumor antigen, and
(b) a second polynucleotide encoding a fusion protein comprising an APC-targeting ligand and an exosomal or lysosomal transmembrane protein.

Embodiment 2. The vaccine composition of embodiment 1, wherein the APC-targeting ligand comprises ICAM1 or ICAM4.

Embodiment 3. The vaccine composition of embodiment 1, wherein the APC-targeting ligand is selected from the group consisting of CD2, CD11a, CD18, CD22, CD29, CD40L, LDL, oxLDL, Lectins, Galectin 1, Galectin 3, Flagellin, Cxcl5, KRT14, FGF7, FGF10, and AMP-IBP5.

Embodiment 4. The vaccine composition of any one of embodiments 1 to 3, wherein the viral antigen is from a retrovirus, reovirus, rhabdovirus, poliovirus, potyvirus, geminivirus, flexivirus, picornavirus, togavirus, orthomyxovirus, paramyxovirus, calicivirus, arenavirus, flavivirus, filovirus, bunyavirus, coronavirus, astrovirus, adenovirus, papillomavirus, parvovirus, herpesvirus, hepadnavirus, poxvirus, or polyomavirus.

Embodiment 5. The vaccine composition of embodiment 4, wherein the viral antigen is a SARS-CoV-2 antigen.

Embodiment 6. The vaccine composition of embodiment 5, wherein the viral antigen is a SARS-COV2 spike protein.

Embodiment 7. The vaccine composition of any one of embodiments 1 to 6, wherein the first polynucleotide and the second polynucleotide are present in a single plasmid.

Embodiment 8. A method of vaccinating a subject, comprising transfecting skin cells of the subject with the vaccine composition of any one of embodiments 1 to 7.

Embodiment 9. A vaccine composition, comprising an extracellular vesicle (EV) comprising a viral, bacterial, or tumor antigen and a plasmid or oligonucleotide encoding a viral antigen, wherein the EV is decorated on the surface with an APC-targeting ligand.

Embodiment 10. The vaccine composition of embodiment 9, wherein the APC-targeting ligand comprises ICAM1 or ICAM4.

Embodiment 11. The vaccine composition of embodiment 9, wherein the APC-targeting ligand is selected from the group consisting of CD2, CD11a, CD18, CD22, CD29, CD40L, LDL, oxLDL, Lectins, Galectin 1, Galectin 3, Flagellin, Cxcl5, KRT14, FGF7, FGF10, and AMP-IBP5.

Embodiment 12. The vaccine composition of any one of embodiments 9 to 11, wherein the viral antigen is from a retrovirus, reovirus, rhabdovirus, poliovirus, potyvirus, geminivirus, flexivirus, picornavirus, togavirus, orthomyxovirus, paramyxovirus, calicivirus, arenavirus, flavivirus, filovirus, bunyavirus, coronavirus, astrovirus, adenovirus, papillomavirus, parvovirus, herpesvirus, hepadnavirus, poxvirus, or polyomavirus.

Embodiment 13. The vaccine composition of embodiment 12, wherein the viral antigen is a SARS-CoV-2 antigen.

Embodiment 14. The vaccine composition of embodiment 13, wherein the viral antigen is a SARS-COV2 spike protein.

Embodiment 15. A method of vaccinating a subject, comprising administering to the subject the vaccine composition of any one of embodiments 9 to 15.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A vaccine composition, comprising
(a) a first polynucleotide encoding or comprising a viral, bacterial, or tumor antigen, and
(b) a second polynucleotide encoding a fusion protein comprising an APC-targeting ligand and an exosomal or lysosomal transmembrane protein.

2. The vaccine composition of claim 1, wherein the APC-targeting ligand comprises ICAM1 or ICAM4.

3. The vaccine composition of claim 1, wherein the APC-targeting ligand is selected from the group consisting of CD2, CD11a, CD18, CD22, CD29, CD40L, LDL, oxLDL, Lectins, Galectin 1, Galectin 3, Flagellin, Cxcl5, KRT14, FGF7, FGF10, and AMP-IBP5.

4. The vaccine composition of claim 1, wherein the viral antigen is from a retrovirus, reovirus, rhabdovirus, poliovirus, potyvirus, geminivirus, flexivirus, picornavirus, togavirus, orthomyxovirus, paramyxovirus, calicivirus, arenavirus, flavivirus, filovirus, bunyavirus, coronavirus, astrovirus, adenovirus, papillomavirus, parvovirus, herpesvirus, hepadnavirus, poxvirus, or polyomavirus.

5. The vaccine composition of claim 4, wherein the viral antigen is a SARS-CoV-2 antigen.

6. The vaccine composition of claim 5, wherein the viral antigen is a SARS-COV2 spike protein.

7. The vaccine composition of claim 1, wherein the first polynucleotide and the second polynucleotide are present in a single plasmid.

8. A method of vaccinating a subject, comprising transfecting skin cells of the subject with the vaccine composition of claim 1.

9. A vaccine composition, comprising an extracellular vesicle (EV) comprising a viral, bacterial, or tumor antigen and a plasmid or oligonucleotide encoding a viral antigen, wherein the EV is decorated on the surface with an APC-targeting ligand.

10. The vaccine composition of claim 9, wherein the APC-targeting ligand comprises ICAM1 or ICAM4.

11. The vaccine composition of claim 9, wherein the APC-targeting ligand is selected from the group consisting of CD2, CD11a, CD18, CD22, CD29, CD40L, LDL, oxLDL, Lectins, Galectin 1, Galectin 3, Flagellin, Cxcl5, KRT14, FGF7, FGF10, and AMP-IBP5.

12. The vaccine composition of claim 9, wherein the viral antigen is from a retrovirus, reovirus, rhabdovirus, poliovirus, potyvirus, geminivirus, flexivirus, picornavirus, togavirus, orthomyxovirus, paramyxovirus, calicivirus, arenavirus, flavivirus, filovirus, bunyavirus, coronavirus, astrovirus, adenovirus, papillomavirus, parvovirus, herpesvirus, hepadnavirus, poxvirus, or polyomavirus.

13. The vaccine composition of claim 12, wherein the viral antigen is a SARS-CoV-2 antigen.

14. The vaccine composition of claim 13, wherein the viral antigen is a SARS-COV2 spike protein.

15. A method of vaccinating a subject, comprising administering to the subject the vaccine composition of claim 14.


SEQUENCE LISTING (58 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3819 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacc

SEQ ID NO: 3 - protein, 957 aa, Homo sapiens
ELLCYSELQPQPRYGSQQPPARAARTPGPARGSVPRTWQCPDICVPLKSHPAPGRLRAGDMQHLLPAQVVGHRDPVAKGVAPAWEQPEGVTEQCARRPTNVLFKLPWAVNSNLPHRVLDSRTGGTGTPPLLAASGQEPYPTLPGGGWGTPGQPHRGAAPWGEGAETGASCGGARGHDHGAGEERSPWSQFLVPHTGPAAPRAGAVEHLGPLPAPDLCPASDSPTTCQPPGPRGGHAGDRGLFPGRAVPSLGGPGPPGTGGPEVEPHSHLWQRLLLGQGLSQCDRRGRGHPAADVCSNTGEPEPGDTADSDHLQLSGAQRDSDEARGLRRDRGDSEVGPPSQGDAEWGSSPATGPEGPAPAEGHPRGQRAQLLLLCNPGGGRPAYTQEPDPGASCPVWPPTGREGLSGKLDVARKFPADSNVPGLGEPIARAQVSKGWHFPTAHRGISDCHSRSGHLPLSGQEHSRGGHPQGDRECALPPVDCHHHCGSSRSHNGHCRPQHVPLPPAEDQEIQTTTGPKRDPHETEHTSHASLNLSRDRASSSAFPYWWQWCHTEQSGRHMPCSYTYRPWDAGGQGIVLSQIQQHLGPWYLHTNTRPRISVVTLSQEEGARLKTLMDVKVPDERGSGGGDIAPPGHTTGKYNLLPIGYAEAPQTYRRSGPPTCVASKHKGPHFLTDASLGTAVYPQPLMICIYSFVILPAIYVSFMAKTVSGLTELPVLITFKVTRYSCTGCTLQESAWQKDQMGLGLLIGQPAFPQKEFFYRHKSTIWTGNGYRFRDYPVRPYSSLPPKTDTFVSHLPTHIHFCQCSQHSAVMSGHECPGNMPKLCLVLLSCLHFTGSLHYAAPVSCSDQGPASSGEGGQGIGGLPPSFGSLIRVCVCVYVTSSRSVTQAGVQWCNHGSLQSPFGLKSSHLSLLSSWDHRLTTPHLANLIFFFFPETGSRNIAQTSFVLVNKAFSTA

SEQ ID NO: 4 - DNA, 2967 bp, Homo sapiens
gagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 5 - protein, 388 aa, Homo sapiens
ISRAGPGSLARGPLVRAFCHGVSVPSVAAVFFGGRLPGSWERAGTPDAGAKPQGPSRALRDLSALLGAHEPGVRGCAAGEVSAAQLQQQLSPAAEFQPPHPAAARQDAQRAGLGVLPAARREGLELPRALPRDLRRKNTLGHLQDHRLQCSRWATWWPGMEAGSSIPKAWSASPAWIWPTPPTSLLLDPATSGSPSATRASISTAWWSATARHPLHCSLGAPRPQLWPPVPSLPLGSSSLWALRTYASALSPRRKGGCSMPAEREKEEYETIWGNGHTWWLTPVIPALWEAEAGESLEPRSSRPAWTTDPVYAKNTQISLVWWPAPVVPATREAELGGSFEPKSRLQALIVPLHSSLGDRARPCLQKNKNKNKYWRGNPLESIKASLT

SEQ ID NO: 6 - DNA, 1213 bp, Homo sapiens
atctctagagccggccctggctctctggcgcggggccccttagtccgggctttttgccatggggtctctgttccctctgtcgctgctgttttttttggcggccgcctacccgggagttgggagcgcgctgggacgccggactaagcgggcgcaaagccccaagggtagccctctcgcgccctccgggacctcagtgcccttctgggtgcgcatgagcccggagttcgtggctgtgcagccggggaagtcagtgcagctcaattgcagcaacagctgtccccagccgcagaattccagcctccgcaccccgctgcggcaaggcaagacgctcagagggccgggttgggtgtcttaccagctgctcgacgtgagggcctggagctccctcgcgcactgcctcgtgacctgcgcaggaaaaacacgctgggccacctccaggatcaccgcctacagtgttcccggtgggctacttggtggtgaccctgaggcatggaagccgggtcatctattccgaaagcctggagcgcttcaccggcctggatctggccaacgtgaccttgacctacgagtttgctgctggaccccgcgacttctggcagcccgtgatctgccacgcgcgcctcaatctcgacggcctggtggtccgcaacagctcggcacccattacactgatgctcgcttggagccccgcgcccacagctttggcctccggttccatcgctgcccttgtagggatcctcctcactgtgggcgctgcgtacctatgcaagtgcctagctatgaagtcccaggcgtaaagggggatgttctatgccggctgagcgagaaaaagaggaatatgaaacaatctggggaaatggccatacatggtggctgacgcctgtaatcccagcactttgggaggccgaggcaggagaatcgcttgagcccaggagttcgagaccagcctggacaacatagtgagaccccgtctatgcaaaaaatacacaaattagcctggtgtggtggcccgcacctgtggtcccagctacccgggaggctgagttgggaggatcctttgagccctgaaagtcgaggttgcagtgagccttgatcgtgccactgcactccagcctgggggacagagcacgaccctgtctccaaaaataaaataaaaataaaaataaatattggcgggggaaccctctggaatcaataaaggcttccttaacca

SEQ ID NO: 7 - protein, 1452 aa, Homo sapiens
GFPRDLEVERRAQQNASRQPPPKQEVAFGRLHINRHHQKEKGRRRQQRLGSCLQFQLQRDTFVASRLGLCREGPEGANRRGAQMSSCTPTPPTLGFVHRPVICFSDLFCIHGEERSEEENKVTPGGSEELWPTPPVPTAAATHVHCSPDWRDAGSGSEDGARTGSRSFLEVMDVGALASGQIRTQRELSRSHHLQLSLCLAAGLTLPVCSFDNFQCREFQYNVEMDTDTLCLSLSPPAGLLLLLLVALFGVFVPCHLPSSWSGHGVTGGHQLLFFLLVLLLSFQCGKACAELQSPPRRCPLEKAVLLHQVLSHDEERQGQRDQERLSVQCPGDNISGNRSCCRQSHQQQLLLSHEQEGETLWLKRVQRLAERENRGKWIQHLCIFLAAQWQANVCGIEWKRSSQERTKNKKEKHLCSLPPHDDPNIEENTVGGCSTTNDSLDRKRWYPHRLLKRQRHSPEFSLFKGRKALDVFVLTADIQSSFHLCVIVSCLIIKIEASQVWMEVILKTMLWWGLGFFCLFVCFIFKFLFLNFDRTRTWNTLLNDLWESYLWNMNTYQRLSLLIQAFNEQDTQAFTGKHLGHIICTTKGALGVAPWKNWIRFTNINIVVNCPNTNSMVCLCILSPSFSISFVIYLIGCISFGVRVSSMLPSGLPFLSLSLSLSLSLSLSLSLSLSLSLSPSLSLPPPSGTIRTFMTKCFKTLANTSEIGDEIKEAVMPHALSVDLICTSAIKNTSNDYGRILLIMQISVIICQHISVQRDPYLKMKAKFSFIVLFHLQEYTKRRKNELLDHCWVFFFLLMMACHRSQWQMMQRLSAHTALCKAHRILLPQKNQKRKFGMKCNSPWGLTANISYMSNHILTPVQAEWSSLYRTTTLFSVNLKCSRRFHNITTLKFIFLEKVKAVCKCAMHPRSVDHINTALFAVSVVILRMETLLKKFRITLRYCCSLLSCLEMSVYFHCLDSVFEQKDSPNVSVYNLTSREASDLSSSNLFVLMLDQYKSAFNIIIKGSVILKLAFFFRMFLQVIIYDFFEKLAFQEKSTEAQCFGLWVVAFCGVLSTCFLTNGIFKMYTSCGWILYVRVLVTESKGSNVMNLKNVSFFTFILKLYLTHDHGPPTTKIKCLGRQTIVFSDKGNSKCRRWILISLCHLDYRDYVTVIIIVLKTLNSFQNVSKLAESKNRKHLSPSPTLHFPLYASQRIQNEFGKERGDSSSLHICLILIENSGPFYNYKKRSSVLNFVCKKKRFCEFCLRGKCGHCSTMLKCVSHGYRAYDCHQTPRYRIASLLLILKRLNSVYYVTFKRQKCWEKRIKVLFLFYFLSFFSSLFFSFLFFFFSFFFLFFFYPKTIPGNEISSRAVCMAGCGMPHSPKQQQQKKKLLKHLGFFNVLHVAGYPYPNNNQMLSGSPNYSGPPSLHSGRRMNKQGWLVQQEALGGKPGAANVDFQGMEKTNEFDLKS

SEQ ID NO: 8 - DNA, 4572 bp, Homo sapiens
ggctttccaagggacttggaggtggagagaagggcccaacaaaacgccagccgccagccgccccccaaacaagaagtggctttcggaagacttcacatcaacaggcaccaccaaaaagagaaaggaaggagaagacaacagcgcctgggcagctgcctccagttctgacaactccaaagagacactttttaagtggccagcaggctgggactctgcagagaaggaccagaaggtgccaaccgcagaggggcgcagatgtcttcctgcacccccaccccacccactttgggttttgttcaccgtcctgtcatctgtttttcagacctcttttgcatctaacatggtgaagaaaggagtgaagaagagaacaaagtaacccccggggggagcgaagagctctggtgaccgacaccaccagttcctactgccgcggccacccacgtccactgttcaccctgagactggagagacgcaggcagcggatccgaggacggagcgaggacaggcagccggtccttcctagaagttatggatgttggtgcactcgcttctggccagatccgtacccagagggagctatccagaagccaccacctccagctgtctctctgcctcgcagcaggtcttacccttccagtatgttccttctgatgagacaatttccagtgccgagagtttcagtacaatgtggaaatggatactgacacattgtgcctcagcctttccccacctgccgggctgctgttgctgcttcttgttgctctttttggtgtcttcgttccctgtcacctgccaagctcttggtcaggacatggtgtcacaggaggccaccaactgctcttcttcctcctcgtccttctcctctccttccagtgcgggaaggcatgtgcggagctacaatcacctccaaggagatgtccgctggagaaggctgttctccttcaccaagtactttctcacgattgagaagaacggcaaggtcagcgggaccaagaatgaagactgtccgtacagtgtcctggagataacatcagtggaaatcggagttgttgccgtcaaagccatcaacagcaactattacttagccatgaacaagaaggggaaactctatggctcaaaagagtttaacaacgactgtaagctgaaagagagaatagaggaaaatggatacaacacctatgcatcttttaactggcagcacaatggcaggcaaatgtatgtggcattgaatggaaaaggagctcccaggagaggacaaaaaacaagaaggaaaaacacctctgctcacttcctccccatgacgatccaaacatagaagaaaacactgttggtggatgcagtacaaccaatgactctttggacagaaagagatggtatcctcactgaagactgtagctcaaaaggcaaagacatagccctgaattcagcttgtttaaaggaaggaaggctttggatgtttttgtactcactgctgacatacaaagttcttttcactagctctgtgtcattgtgtcatgccttataatcaagatagaggcaagtcaagtttggatggaagttatcctcaagtgaacaatgttgtggtgggggctgggctttttttgtttgtttgtttgtttcatttttaagtttttgtttttgaacttctgagatagaacttaaagaacatggaacactctgttgaatgatctttgggaaagttatttatggaatatgaacacatatcaaagactttcattgctcattcaagcctgatgattcaatgagcagtaagacacgcaagcatttactggaaagcacttgggtcatatcatatgcacaaccaaaggagctttgggtgtggcaccatggaagaattggatcagatttacaaatataaacatagtagtatgaaactgtcctaatacaaatagtatggtatgcttgtgcattctgtctccatccttttctatttccttctaagttatttatttaataggatgttaaatatcttttggggtttaaagagtatcttcaatgctgccctctggtttaccttttctctctctctctctctctctctctctctctctctctctctctctctctctctctctctctctctctctctctccctctctctccctccctcccccctctggcaccatacgcacattcatgacaaagtgttttaaaaccttggcaaacacttcagaaataggagatgagatcaaggaagcagtatgaatgccccatgcgctctcagttgacttaatttgcacttctgcaataaaaaacaccagcaatgactatggcagaattctgctatagattatgtaacagatatctgtcatcatttgtcaacatatatcagtccagagggacccttaccttaaaatgtagaaggccaaattctctttcattgtcttatttcatcttcaagaatatactaaaagaagaaaaaatgaattgttagactaacattgttgggttttttttttcctactgatgatggcttgccacaggtcacaatggcaaatgatgcaaaggttatctgcacatacatgagccctttgtaaggcccacagaatccttctccctcaaaagaaccaaaaaaggaaatttggtatgaagtgcaactctccctggggcttaacctgagcaaatatatcctagtatatgagtaaccatatactgacacctgttcaagctgaatggtctagtctttacagaaccacataaaccttgttttctgtaaatttaaaatgttctagaaggttccataatataaccacattgaaattcattttcttagaaaaggtatagaaagcagtatgtaagtgtgccatgcaccctcgctctgtagatcactaaataaacacgtaagccttatttgcagtgtctgtagtgattttaagaatgtaggaaacacttctaaaaaaattttaaaggataactctgagatgatattgatgctgcagtcttctttcttgtttggaaatgtctgtttattttcattgtttggattcagtattttgataggaacaaaaagactcaccaaatgtgtctgtttactaaaatttaacctctagagaggctagtgatttgtgatcctcttctaacttatttgtgctgatgcttgaccagtacaaatcagctttttaaaatattattattaaaggttgatcagtcattttaaaattggccttttttttcagaatgttcctacaggtcataatttatgatttctttgaaaagcttgcatttcaagagaaaagcacagaggcacaatgctttggtttatgggtataggttgcattttgtggtgttctttcaacttgttttctgacaaatgggatttttaaaatgtatacttcttgtggttggattctgtatgttagagtttaattggtaactgagtctaaaggctctaatgtaatgaatctctagaagaactaggtatctttttttacttttattttaaaataataattatacctgacacatgaccatggaccacccacaaccaaaattaaatgtttggggagacaaactatagtattcagtgacaagggtaacagcaaatagtgcagacgttggattcttatttcactttgccatttagattactaaagagactatgtgtaaacagtcatcattatagtactcaagacattaaacagcttctagcaaaatgtatcaaagcttgcagagtccaaaaatagaaaacatctttccccctctcccaccctacatttccccctgtatgcatcctaacagagataaatacaaaatgaattcggtaaggagagaggagattcttcttcacttcatatttgtttgatattaatagagaattctggtcctttttacaactactgaaagaaaagaagttcagtcctaaattttgtgtgttaaaaaaagaaaagattttgtgagttctgcctccgtgggaagtgtgggcactgctccaccatgctgaagtgtgttagccacgggtacagagcatatgactgttgacatcagactccttaaagatacagaatcgcttccctcctcctaatcctcaaaaggctgaacagtgtatattatgttacatttaaataaaggcaataaaaatgctgggaaaagagaataaaagtactgttcttattttatttcctttctttcttctcttctcttttcttttctttccttttcttttttttttcctttttttttcttttttttttttattagcctaaaactatacctggtaatgagatcagctccagggctgtgtgcatggcaggatgtggttaaatgccccacagccccaaacaacaacaacagaaaaaaaaattactcaaacatttgtaaggtttctttaatgttttacatgtgtgagccggctatccttaccctaataacaaccaaatgctttcgggttctcctaactactcaggtccacctagtttacacagtggataaagaagaatgaattgaaaacaaggatggcttgtgcaacaatgagaggctcttggaggaaagccaggagctgcaaacgttgacttccagggcatggaaaagaccaacgaatttgatttgaaaagt

SEQ ID NO: 9 - protein, 1664 aa, Homo sapiens
THTHKHTRAHTQRENPSACFMETIMILLENFSAEKFVATVERLKLHQADNRHGILIYPAVSNKTKVKQTASQQLNLLRTVFMRIYQQSYLRRNPVLLSGTKRIRLTIWKEQLLFLKSIYNSQIGRGQPRSNNQLKIHFHYVIHEHPEHYTIMHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITLHMVYKEPVPAGRFLVDCFLSSQNFLSFYFLVIKKGWKTTEKLIKLDLCIYVCFKTLHRKIKVYTKIRFSNRLKIVKLVVQSCQFFSINLPLRVCILSDNDYLNIPICLNGCYNNNNTDVVIGISDLQASGRICQIIKPHLWKMSSILNAFKIIIYLNSNQKKNSQKNYYESQNRFNKSTGLEHAYTYKEQNFCCSSGKGIAKRMFPKILYIRQQLMIILYFILLATKHFINPQSKIEKSLSFFQVTSIFVFIFGNMAFNNVLPTNNHAFFLWLQHTLFVVFELYCFVIVYVIYKKKTLISCICFICFFRNNKTVWLNGKFPSLFLTLSLAHSTWASKSWKADKNKSLKQCLQMSHTEQYKYVKNLSPHILANLDHISKIITNISIYRILKLEYIMHGKISYFKTMYIIFSNLKSLLFKRWQHKLFSCMARAQCPKKMQLRIRGPECHEGLRSAYRQDYYKDEFPLQKSFIGRSWHFICSPMGGQYLSNLKGMLTTVVLISKYLSFKSLYINSIWYIYRELYEKARSTKTIDSFNFPVVDLYDQDVENKELPFLRYTLGREHEWYSELSPDSRTLLARFGQASVTVVLAYGQRRTFFICVSLVPSRLSVHNQFSVIHMFIDKRKYTLKLIFILKGNMTIQKFKKCIYINTSYVRTKCSLSMELYFHQITQCIRQNQHLVVNPFLVVVTKRLLEICTLIICFSPIFKFIMQILENKICSSYTPLEFNIKTVRGLHPSYSFVVEIEILIHLRSRSWVDIQLSQESRSVSTCFLLFDYYYLMVFGMFLFFISVEIPLHLYTNVTMSWQCTSYTMTNLYCDDLTQKEKRNYVVFNSDSYSPFVYEWKALCKIYIAEAFKCSLKDKIKYMSFQLEKKKLKHEMITKVNKRYFQSSEQNILTAIIIIIKIKTIVIVSLVGYKFNLMTEEKGCWRILRVSISSLLFWFSRLPKNIKMCEGTLKQSYNKKNSKTVPLTIFYNFSNSRGVKEVRECKGGPPQTEHFFSLKTHVIFASYSIIYLWLRYDNVCNTPVSKIDTSELAGLIMQFGLHPYSTAIYLSVNETQKIICLPSNFTHKHKAKENQNSNSPRIGVITKIFNSQYGLLANQNTSLIHRNGCRPLLTCGYHFYDHPKTELGKYLLTGLSGLNSQYVSYEIAHNFMCNYFSIVVSYSLFHFCTVFIQPSCCFQRSLVPSGTHILPPILFMVCQENLLSLELKNLTGSKKYSPSLFPITSDFSILKLHFSRNQIFTLIWEDASYVFLLLPGYHVVAFSKFLSLNLEPIKIYFLSFIFLKCEMKITFHLKTLLLMNPSMCLSIAVQIKTWLET

SEQ ID NO: 10 - DNA, 5320 bp, Homo sapiens
acacacacacacaagcacacacgcgctcacacacagagagaaaatccttctgcctgttgatttatggaaacaattatgattctgctggagaacttttcagctgagaaatagtttgtagctacagtagaaaggctcaagttgcaccaggcagacaacagacatggaattcttatatatccagctgttagcaacaaaacaaaagtcaaatagcaaacagcgtcacagcaactgaacttactacgaactgtttttatgaggatttatcaacagagttatttaagaggaatcctgtgttgttatcaggaactaaaaggataaggctaacaatttggaaagagcaactactctttcttaaatcaatctacaattcacagataggaagaggtcaatgacctaggagtaacaatcaactcaagattcattttcattatgttattcatgaacacccggagcactacactataatgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaattgcatatggtatataaagaaccagttccagcagggagatttctttaagtggactgttttctttcttctcaaaattttctttccttttattttttagtaatcaagaaaggctggaaaactactgaaaaactgatcaagctggacttgtgcatttatgtttgttttaagacactgcattaaagaaagatttgaaaagtatacacaaaaatcagatttagtaactaaaggttgtaaaaaattgtaaaactggttgtacaatcatgatgttagtaacagtaatttttttcttaaattaatttacccttaagagtatgttagatttgattatctgataatgattatttaaatattcctatctgcttataaaatggctgctataataataataatacagatgttgttatataaggtatatcagacctacaggcttctggcaggatttgtcagataatcaagccacactaactatggaaaatgagcagcattttaaatgctttctagtgaaaaattataatctacttaaactctaatcagaaaaaaaattctcaaaaaaactattatgaaagtcaataaaatagataatttaacaaaagtacaggattagaacatgcttatacctataaataagaacaaaatttctaatgctgctcaagtggaaagggtattgctaaaaggatgtttccaaaaatcttgtatataagatagcaacagtgattgatgataatactgtacttcatcttacttgccacaaaataacattttataaatcctcaaagtaaaattgagaaatctttaagtttttttcaagtaacataatctatctttgtataattcatatttgggaatatggcttttaataatgttcttcccacaaataatcatgcttttttcctatggttacagcattaaactctattttaagttgtttttgaactttattgttttgttatttaagtttatgttatttataaaaaaaaaaccttaataagctgtatctgtttcatatgcttttaattttaaaggaataacaaaactgtctggctcaacggcaagtttccctcccttttctgactgacactaagtctagcacacagcacttgggccagcaaatcctggaaggcagacaaaaataagagcctgaagcaatgcttacaatagatgtctcacacagaacaatacaaatatgtaaaaaatctttcaccacatattcttgccaattaattggatcatataagtaaaatcattacaaatataagtatttacaggattttaaagttagaatatatttgaatgcatgggtagaaaatatcatattttaaaactatgtatatttaaatttagtaattttctaatctctagaaatctctgctgttcaaaaggtggcagcactgaaagttgttttcctgttagatggcaagagcacaatgcccaaaatagaagatgcagttaagaataaggggccctgaatgtcatgaaggcttgaggtcagcctacagataacaggattattacaaggatgaatttccacttcaaaagtctttcattggcagatcttggtagcactttatatgttcaccaatgggaggtcaatatttatctaatttaaaaggtatgctaaccactgtggttttaatttcaaaatatttgtcattcaagtccctttacataaatagtatttggtaatacatttatagatgagagttatatgaaaaggctaggtcaacaaaaacaatagattcatttaattttcctgtggttgacctatacgaccaggatgtagaaaactagaaagaactgcccttcctcagatatactcttgggagagagcatgaatggtattctgaactatcacctgattcaaggactttgctagctaggttttgaggtcaggcttcagtaactgtagtcttgtgagcatattgagggcagaggaggacttagtttttcatatgtgtttccttagtgcctagcagactatctgttcataatcagttttcagtgtgaattcactgaatgtttatagacaaaagaaaatacacactaaaactaatcttcattttaaaagggtaaaacatgactatacagaaatttaaatagaaatagtgtatatacatataaaatacaagctatgttaggaccaaatgctctttgtctatggagttatacttccatcaaattacatagcaatgctgaattaggcaaaaccaacatttagtggtaaatccattcctggtagtataagtcacctaaaaaagacttctagaaatatgtactttaattatttgtttttctcctatttttaaatttattatgcaaattttagaaaataaaatttgctctagttacacacctttagaattctagaatattaaaactgtaaggggcctccatccctcttactcatttgtagtctaggaaattgagattttgatacacctaaggtcacgcagctgggtagatatacagctgtcacaagagtctagatcagttagcacatgctttctactcttcgattattagtattattagctaatggtctttggcatgtttttgttttttatttctgttgagatatagcctttacatttgtacacaaatgtgactatgtcttggcaatgcacttcatacacaatgactaatctatactgtgatgatttgactcaaaaggagaaaagaaattatgtagttttcaattctgattcctattcaccttttgtttatgaatggaaagctttgtgcaaaatatacatataagcagagtaagccttttaaaaatgttctttgaaagataaaattaaatacatgagtttctaacaattagaaaagaaaaaattaaaacatgaaatgataacaaaagtaaacaaaagatactttcaaagcagtgaacaaaacattttgacataagccataatataaattataatataaaaaataaaaaccatagtataaattgtcagcctttgagttggctacaaattcaatttaatgacagaagagaagggatgctggaggtaaattcttagggtttctatctcatagagtttgctcttctggttctctagactgccaaagaacataaagatgtgcgaggggacctagctgtagtaaaagcaatcctataacaagaaaaactctaaaacagtgccccttacgattttctactgaaatttctctaatagtagaggtgtaaaataagaagttagagaataatgcaaagggggcccaccacagacggaacatttcttttctcttaagactcatgtgatttttgcatcttactccataatatatttgtggttgcgttaatatgacaatgtctgcaattaaacaccagtaagcaaaattgatacatcagaatgacttgcagggcttatcatgcagtttggtttacatccctactccactgccatttacttgagcgtgaatgagacacaaaagattatttgcctcccataatccaactttacacataaataacacaaggctaaagaaaaccagaactcaaattcaccacgcataggagtgataacaaaaatatttaacagtcagtatgggtgattactggccaatcagaatacatcactgatacatcgaaatggatgcaggccactatgactaacttgtgggtatcatttctatgatcaccctaaaacagagttgggaaaatatctattaactggtctctctggtttgaattctcaatatgtatcttaatatgaaatagctcattaaaacttcatgtgtaactatttcagcattgttgtcagctactctttattccacttctgtacagtatttattcaaccaagctgctgctttcaatgaaggtcacttgttccttcagggacacatatactcccacctatcctttaattttgaatggtttgtcaggaaaatttactttctcttgagttgaaaaacttgacaggaagcaagaaataatacagtcctagcctctttccaataacatctgatttctccattctcaaactacacttctcaaggaaccagatatttactctcatctgggaagatgcctcttatgttttccttttacttcctggttatcatgtggttgcattttccaagttcttatcattgaatttatgagagcctatcaaaatttattttctttcatttatattctaataattgaaatgtgagatgaaaataacatttcacttatgaaaaacccttctcttgatgaatccttccatgtgttagttatctattgctgtgtaacaaattaaaacttaatggcttgaaacaaa

SEQ ID NO: 11 - protein, 529 aa, Homo sapiens
TRAPSLHSANCSLAHLPPLHHDHLQPPVHLLQLHEGLLRHRGRHRGRLQPHLLRPGRRVLPRPQHLRGRPVCLILPLLLWGSLRAGGRLWRWLQQQQQQLWWLWGRIWWWPWCWLGWWLWWWLCWWWASGGQEGDHAEPQPPGLLPGQGACSGGGQRRPGSEDPLVPEAAACDQRLQSLLQDHGPEEQDSHSHSGQCQCPSADQCPSGRGLPHQVDRVEPAHECGSRHQWPAQGAGRTDPGQSPGDADEPEGGAGLPEEEPRGGDECPERPGGWRCQCGDGRCTWRGPEPHSERDAPVEDGREEPQGCRGMVLHQDRGAEPRGGHQQRAGAERQERDLGAPAHHAEPGDAAVPAQHESIPGEQPGGDQRSLLHAAGPDPGDDWQRGGAAGPAPLRDGAAEPGVQDPAGREDAAGAGDRHLPPPAGGRGRPPLLLPVLLWIAVIQRCDLLQPPNPHQGHGCARWQGGVHPRAGPSHQELRLPSPAQAEAPRVDTDPTGRSPLLPKHFTAGPCFTLTPSWQSIQLHYLSCI

SEQ ID NO: 12 - DNA, 1636 bp, Homo sapiens
acccgagcaccttctcttcactcagccaactgctcgctcgctcacctccctcctctgcaccatgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctgcgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcattatctgagttgcataa

SEQ ID NO: 13 - protein, 511 aa, Homo sapiens
SLTSVPFARAQNQKRKPTPKMSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNKRKLSFSIKSTVDFCPPDVHIRTSMRCFLCAEHCHLLRLWATATSASSNSAMWSTSGVFGLLRELHHTSKEKQYKCDCKNGRGPSTEILEISCPLSGHVMRIKLVCLGLTTSSLSAETLEFLMCPGGHLPTILVKVKKLL

SEQ ID NO: 14 - DNA, 1565 bp, Homo sapiens
agtctcacttcagttccttttgcatgaagagctcagaatcaaaagaggaaaccaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactaga

SEQ ID NO: 15 - protein, 1060 aa, Homo sapiens
VFEAPKNISCCWEYEDIDVECVLVCWRCINDSTCQTTRGSLFHDFRAVVSFFPLGREANKLSHRLWRGTTLQYWLYICRSSLKDRYLSPGCELTQQSGRGPQYHSACYLKRGNGQSKAQQSDCSNTFKIGADCSWVCKPNFICVWIKKANVHGGENELNFPCYQHWQYGSQCCGNNGTKFFPPNAVQHFGCPDYYWRMPLKLSKSVCIRAAKECNADLERHSPVLVQDEAIVLHKSSTLFKFLVFWENGKWKRSQCSYPTGRPAIHFRNGDFSTQVNKSNRFSRAKSKSNTKQGECCACSTGRTTSSKTQTLFHHSDYFKLATWTYCTSVDLICYVEGWLLKTIQIYPTRRKQKRQLELYQQKQLRTSFKLREWKTDSGCSKEIKTLFTRKNEFCLDFFYSSCDILCLHARGKSQQLLFEIEELQRYSQRSLSFMGRETLKHSIYSRKVSPRYLEMKVLSIILEKSTNTTYHMCLPQNEPHWVGRGSFQIHLYLFKICSLKIFFRELFPNFLTSGPLSLSPLFIIHFLRAVFQQPFFFSRLILYYRPNWQTSDTCTLVANDFWIIIFLLWISPSFFLYIYMCFYVGIYLPFFLSILPITHLYQAYPGVIFKSFVIFNKRLVLHFTYMLIKIQKPYLTNNFKIKAVQRLGGLYFICIMYYVKYLSHNYFLGCNSLLPFTSISVTLHGNEETKFINLNSILKDTDSICIQIMRRQQNFHEMNIFIVCSYYMRFYFKLSGFKISLNTIIFVIFILCLSRLQNIILSDSVFIRELWPNFEHLLGVIKLEGNVEKQFWERFLYMKSLPLASHPNKLSVHRPAVMVRNVLRFAKAFENVSTKPPEPRVCIHRNKLYDIYVFLKNFVSLKGSFFQENIDHRKIKPRFRTVFSRIVSKEIYIWFFPTQNYSETTIYFRLFEHSESRVLWLSNTGLSDTRGTQLHIGLDFAQFKIVFVINLLCYLYHEFNPTTLRRQGWVILFWQVGYITMFNLTALDGAVNQGRLMKSVLYLQNIGNLTLMSSNPEKKNPNYFRIIVKYCIMMVAKFFVCPINTLKKEFELIFKKKKK

SEQ ID NO: 16 - DNA, 3459 bp, Homo sapiens
gtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctgttgtttcatttttcccattaggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccagttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagactcaggttgtagtaaagaaatttaaaagacactgtttacaagaaaaaatgaattttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagactgaacatgtacactggtttgagcttagtgaaatgacttctggataattatttttttataattatggatttcaccatctttctttctgtatatatacatgtgtttttatgtaggtatatatttaccattcttcctatctattcttcctataacacacctttatcaagcatacccaggagtaatcttcaaatcttttgttatattctgaaacaaaagattgtgagtgttgcactttacctgatacatgctgatttagaaaatacagaaaccatacctcactaataactttaaaatcaaagctgtgcaaagactagggggcctatacttcatatgtattatgtactatgtaaaatattgactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgtcttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggattgtctctaaggaaatttacatttggttctttcctactcagaactactcagaaacaactatatatttcaggttatttgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagttttttgtgtgtccaataaacacattgtaaaaaaaagaatttgaattgatatttaaaaaaaaaaaaaaa

SEQ ID NO: 17 - protein, 1691 aa, Homo sapiens
IIFLFHPVVASKSHGPPDAAPGATGPSSAGRMKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKDVQACEVQSAQNWTQDAQGHSASACILPCALGRVTASPWPSVSLSRTWNSFLPVSFAGSGRPAEGPAKRAAKVRACHYQTVHQPLLVSFLGRECLIMWRNCSLRTGCSGPHPCPGMSTDASTPQNLSLHTPLHWSPVSSAGRKQMPVSLRDCGTRLVLGQRPNSLACLPAPCKMRPSWPSPASSRAVMPPCSSGDTSLSPRPGSFLSSCIHPDSSLCLNLPSRHPSFLDQQIPAHVTLGWVLTSFTLPPACTTPSKHTSCFFIRQPGCFFPVLTYLAAISQTVRVKAILVLFTLGCLMICQGVGHLVGAHNFTELMTEPVGRYRKRGAGLGAVVHACNPSTLGGQGGWITGQELEASLAKPHLYKYKIQAWWHTPVVPATQEVEVGELLEPGRWRLQAKIAPLHSSLGNTARLRLKEKIKIKSGHGPVTSPPLEAVFSGSALPLHTLSQDPSRLCSGPHRKTELKVGTTSANLEPQCQAQCLHVFIQMNSV

SEQ ID NO: 18 - DNA, 5129 bp, Homo sapiens
atcattttcctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggaatgaccttggcaacagaccccacagatggaagcattttggcctgtgaccctgggctgtctcgaacgtgtgaccagaacacctatctgagtggcctgtgttacctcttccgccagaatctgcagggtcccatgctgcaggggcgccctggttttcaggaatgtatcaagggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaacggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcttggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctcatcccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgcagggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccaggcctgtgaggtgcagagtgcccagaactggactcaggatgcccagggccactctgcctctgcctgcattctgccgtgtgccctcgggcgagtcactgcctctccctggccctcagtttccctatctcgaacatggaactcattcctgcctgtctcctttgcaggctcatagggaagacctgctgagggaccagccaagagggctgcaaaagtgagggcttgtcattaccagacggttcaccagcctctcttggtttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgctggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgccacccctccttccttgaccagcagatcccagctcacgtcacacttggttgggtcctcacatctttcacacttccaccagcctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgatgcctcatgatatgtcagggcgtgggacatctagtaggtgcttgacataatttcactgaattaatgacagagccagtgggaagatacagaaaaagaggggctgggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccgtgacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtcca

SEQ ID NO: 19 - protein, 589 aa, Homo sapiens
NPEGGHFDSLLMLPLPPSLPEDTISTLTQHDRNIQPNFSPICGHWTAHQHENFYVFTYCFSYHPDDWVSTFCCVSSKVGQDRRKESSRFCIHENDTEMQHRRKILILTELGDKPVRLCEGYNVKQRGDEERKQLNAKRSESSNCGTCHKGQQNNICVTVGKRILHHEQQLGNPGKWETADRKTRTLLYLCPSHLLFQSGSFESSSIYSQPLPKVPRIRENLTQSCKYPQFRQTLRATIHSLGRSIIATRCFGVCQCDSKPSEPWHWLHVLWLTQTLNSVTLQAVVELTLGVFIIQHSGAHPLLTAYLPDPPYGELFIIHSKACRTVISELQVTNQNGPCSIRAYISEAATPLMQTSRESYEKTRPLCTGILSKQQITCQVQFCFFACSVFPWIMHLIYQRCRREMGSLSSHSVMVDSGFLWPCWRGPGSRTSNTVENRNPPPPPATLSDSYSFSFNLSLSISLFQSLSLNLFLPISLSQSLCFPLSVSSLPQSLFSIPLSNTHTHTHTHTHTHTHTHTESGRCSVLFFPPCPYLYHYRGGVGSAALSLPTPHYEMTVFKGNLLYLPAVSIVSRVNLLSCYLFFERPLNI

SEQ ID NO: 20 - DNA, 1852 bp, Homo sapiens
aatcctgagtaaggtggccactttgacagtcttctcatgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatta

SEQ ID NO: 21 - protein, 770 aa, Homo sapiens
TVLRILQSSLLQSPLLHPVQEPATARSALLTTMSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKENLREMSTHGKVSQSSAEKFSGGLTQGRQEGKILLLFVYLFFQLAFFLDSSLRVGKPMFAAAFSSANEVFSIVPLLFAVILSAMLLKFWQLTIVARNHWLLIFQSVLNCRLLYFQEIFLKILTEKAVDLMWKCFIRILLMEIHCYLHFYKKEIFCFLGNMLENFLTLDCGILFNYFTLESVFHTLSMNIFPYSEFLKVVLGLISYLPIILDILYLFSMANCHHLLLNFDFICYLLSILLGVPFWLNIYFRMKKLENRQIPDCFIKCILLVFKVKANLTMTCTLKVLETYSNNLNINLSFSYKNIRHPRGPSISPWIGDQRELGMLKTKQNKKKQGEVVQGMSIFYPSVWVRFSKIIIRRPAFMVEYIIIYKVATLGQVPSPLTALAPFTENLGRIAEDERERGGQGRCLSGFHSSFHWDFEAFLSECKACSSPGGTHWGWGWGKMRNRLVSVVLISLIMLSLFLQIFLFKLFHLCLEILPFRENVTLVKRLVGKLLPFFLTFKQTVINGCEFLFLLCFTFVFQNRILYLNGKNKTFCAINKATARKIKHFLVKTYVFIYYIFIYNIYYIFSIAELFRCLLCIFRFPFCYELHIYYIHYIKIVLFYYVSHWFIVFILSFETLKDFTS

SEQ ID NO: 22 - DNA, 2436 bp, Homo sapiens
acagtgctccggatcctccaatcttcgctcctccaatctccgctcctccacccagttcaggaacccgcgaccgctcgcagcgctctcttgaccactatgagcctcctgtccagccgcgcggcccgtgtccccggtccttcgagctccttgtgcgcgctgttggtgctgctgctgctgctgacgcagccagggcccatcgccagcgctggtcctgccgctgctgtgttgagagagctgcgttgcgtttgtttacagaccacgcaaggagttcatcccaaaatgatcagtaatctgcaagtgttcgccataggcccacagtgctccaaggtggaagtggtagcctccctgaagaacgggaaggaaatttgtcttgatccagaagccccttttctaaagaaagtcatccagaaaattttggacggtggaaacaaggaaaactgattaagagaaatgagcacgcatggaaaagtttcccagtcttcagcagagaagttttctggaggtctctgaacccagggaagacaagaaggaaagattttgttgttgtttgtttatttgtttttccagtagttagctttcttcctggattcctcactttgaagagtgtgaggaaaacctatgtttgccgcttaagctttcagctcagctaatgaagtgtttagcatagtacctctgctatttgctgttattttatctgctatgctattgaagttttggcaattgactatagtgtgagccaggaatcactggctgttaatctttcaaagtgtcttgaattgtaggtgactattatatttccaagaaatattccttaagatattaactgagaaggctgtggatttaatgtggaaatgatgtttcataagaattctgttgatggaaatacactgttatcttcacttttataagaaataggaaatattttaatgtttcttggggaatatgttagagaatttccttactcttgattgtgggatactatttaattatttcactttagaaagctgagtgtttcacaccttatctatgtagaatatatttccttattcagaatttctaaaagtttaagttctatgagggctaatatcttatcttcctataattttagacattctttatctttttagtatggcaaactgccatcatttacttttaaactttgattttatatgctatttattaagtattttattaggagtaccataattctggtagctaaatatatattttagatagatgaagaagctagaaaacaggcaaattcctgactgctagtttatatagaaatgtattcttttagtttttaaagtaaaggcaaacttaacaatgacttgtactctgaaagttttggaaacgtattcaaacaatttgaatataaatttatcatttagttataaaaatatatagcgacatcctcgaggccctagcatttctccttggataggggaccagagagagcttggaatgttaaaaacaaaacaaaacaaaaaaaaacaaggagaagttgtccaagggatgtcaattttttatccctctgtatgggttagattttccaaaatcataatttgaagaaggccagcatttatggtagaatatataattatatataaggtggccacgctggggcaagttccctccccactcacagctttggcccctttcacagagtagaacctgggttagaggattgcagaagacgagcgggagcggggagggcagggaagatgcctgtcgggtttttagcacagttcatttcactgggattttgaagcatttctgtctgaatgtaaagcctgttctagtcctggtgggacacactggggttgggggtgggggaagatgcggtaatgaaaccggttagtcagtgttgtcttaatatccttgataatgctgtaaagtttatttttacaaatatttctgtttaagctatttcacctttgtttggaaatccttcccttttaaagagaaaatgtgacacttgtgaaaaggcttgtaggaaagctcctccctttttttctttaaacctttaaatgacaaacctaggtaattaatggttgtgaatttctatttttgctttgtttttaatgaacatttgtctttcagaataggattctgtgataatatttaaatggcaaaaacaaaacataattttgtgcaattaacaaagctactgcaagaaaaataaaacatttcttggtaaaaacgtatgtatttatatattatatatttatatataatatatattatatatttagcattgctgagctttttagatgcctattgtgtatcttttaaaggttttgaccattttgttatgagtaattacatatatattacattcactatattaaaattgtacttttttactatgtgtctcattggttcatagtctttattttgtcctttgaataaacattaaaagatttctaaacttca

SEQ ID NO: 23 - protein, 672 aa, Homo sapiens
TRKQACTQTRHHASPRPLAPAPGSRILGFLLKMGFAPNPLRLGGGLRLDPLHLQSPRWPGKLHPVPQSVQEHLEVWDKTLKHKGWEGSFAEKGAIPGRQEELHTEYPPGAPQQWSAGAEDGVQDEMDGTNTPQCLKAFSTSYPAPSRNSRVPGSHSDLLAEFLLLWVSDPIAVAPRGGSNEAGCCHLDLLDHQVCLHPERAQVLPTVESPWEDCDLPASGCRWEVPLQHGAAEREASSQEGDHSDSKPHADSRRRHSDPFLLQFQPQCYPVMETPWRLGGAIAWGAEDPKRWLGQHNHRLRSLLVLVGLPCRPECPVCPPRREGPENQAPFRDSLWKLGQPPMLLKQPPQRSPVLLGEKWQASGERKPAEFLHLPRRCWELQLLGEQLHRTDSVQGLDTSAVCTQEAACVHEPGGPSDGGEECNPDLERRQPSRLPLHLVLEPKPPLPQPEAEIGAGEGPALGCLLVPGDQQCGQGPFASQHPHRLLPGDHRQASGCGTRVLPRHPHPGNLWAQAPATLEEDTEPAGASGEFQRPELLCEEKGKGPPLRPPLPGMLQSNDGRWHLHHPALSRDEHTTNWRCRVLRDAETSPGLRHGHLFSIAQAPSGHYENVIPDFPEDEGIHYSELIQFGVGERPQAQENVDYVILKHHWMGCSRGTGGSGGQGSPRVFPR

SEQ ID NO: 24 - DNA, 2116 bp, Homo sapiens
acgcggaaacaggcttgcacccagacacgacaccatgcatctcctcggcccctggctcctgctcctggttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggcttcaggagaattccagcggccagagcttctttgtgaggaataaaaaggttagaagggcccccctctctgaaggcccccactccctgggatgctacaatccaatgatggaagatggcattagctacaccaccctgcgctttcccgagatgaacataccacgaactggagatgcagagtcctcagagatgcagagacctcccccggactgcgatgacacggtcacttattcagcattgcacaagcgccaagtgggcactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagttttccccagac

SEQ ID NO: 25 - protein, 304 aa, Homo sapiens
ARSTSSPAAVRSQPTSGKWQTIFRSMMRYLGLETQTLKDGLAHGGTSLLGQGATQGLPILGPTPGRHPQGLILDRHLQAPTLEHLELIPEHLHLESTQGHPAALGPTHLLDSQVPPEPTLPLAPMAPLLGHLCLITCLCLGEWCLACQFWARSPMQTELLISKEGMMLPSTLTHASMRTTGESLFAIQSWIITGEGKKDSRFSHLKVGNHSKYKYWLNLTTSRLQMMLTCCSTIIGLKNSMKSANWEFLVTTSPVLHIPYNLKGADKKKKNLNLTCVKVSCSLVKIFTFINIPLVSHLLNKYYS

SEQ ID NO: 26 - DNA, 943 bp, Homo sapiens
gcccgcagcacctcctcgccagcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgccaccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaaaagaatctaaaccttacatgtgtaaaggtttcatgttcactgtgagtgaaaatttttacattcatcaatatccctcttgtaagtcatctacttaataaatattacagtg

SEQ ID NO: 27 - protein, 171 aa, Homo sapiens
ISLGWSLLTAGAPAREHPPGLNHGLWSGRQQPESQTWRVPSSARRGGSRELRAEPGQRQQQPVPALQPSLQRPRRRQHHRVQQQGRRGLGDRAAGGCLSLPAWKCCRGVHHLRPGQPDRQAARWIRIQVPQPPQPGGHQLHGSRLQDQMCGLLKSASPWPPIKAAASAPSE

SEQ ID NO: 28 - DNA, 528 bp, Homo sapiens
atctctctcgggtggagtcttctgacagctggtgcgcctgcccgggaacatcctcctggactcaatcatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgaaatcagccagcccatggcccccaataaaggcagctgcctctgctccctctgaa

SEQ ID NO: 29 - protein, 633 aa, Homo sapiens
VQLLGFLWLPSLLEPGLHPSNHVQGTCSWYSWHHLLLCGCFPARKSRDNCQSGKPNHSKLCRLYGHTVDRCRKESSCNEPHQHSFCQTSDWTQICCCPVYETLALYGGECWQAQGPSRIQGRDQKLLSRGGVFYGSDKDEGNCRSLPWEDCYQCCGHSASLLLSASGYQRCWNYCWSQCTNYANCCCYCLRLRQKGWSRKKRAHLPGRWHFCVNPHYGWNLGQVYSWRHPLGWRRFQPNGQPFYCVAQAEGHQEQESCKTPPYCLTCAYPLFQHPGQYDRFSLRNRLLYLHYPCPIRTECPVPWHPGPSRESPSRCQTRQVTDSYCPGWWFYSYPQDSEASPRLLQWKRTEEHQPSCCLWCSCPGSHLVWRQVECSRFAALGCHSSFPWYNCWWSHDCPHQAYHHSYQADTDLHYLFQPAWCAYSGLRRACHDKGQPAWQVTHRHTSCTPRCSSDSHFHCQWYTQCLCCGQEYGKREQDYYHQGPFEQGRHTYGPGSEVQSREAEGQGVIQEFTVLCLQHESNCRETSRQDRGQTEDSGQVNYQLAESDCEGRITSTERAGESLQPHHHQAVPECRRHARRNAWGISWWWSSSLWWCFLRAHHRGLSQPSVDVALFHTFKTFEGPKFVANSVA

SEQ ID NO: 30 - DNA, 2085 bp, Homo sapiens
gtgcagctcttgggttttttgtggcttccttcgttattggagccaggcctacaccccagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttgatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcagactgctgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggca

SEQ ID NO: 31 - protein, 407 aa, Homo sapiens
DQPTAACICIQRQVPPVPAARAPQSRTRSAQAKLALTMPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVSQLTSLSRKVYPRLVGFFVCLFCFVCCLLFVFLPLILVFILHCIKAEVTLCLSFNASFNIDICSAGGLVCFGLYFFSCLFLLVILSRNPAMKGTIFARLTRYCTKNFFVFKIQMSINFNQVVTYIEDNLIHNKKLQCQKKKK

SEQ ID NO: 32 - DNA, 1246 bp, Homo sapiens
gaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcaaaaaaaaaaaaaaa

SEQ ID NO: 33 - protein, 238 aa, Homo sapiens
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 34 - DNA, 714 bp, Homo sapiens
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatg

SEQ ID NO: 35 - protein, 487 aa, Homo sapiens
GQRASAQRRRRRRPHRASCQASGAQRPTRPRAPAPPRPFLRAPAPRPARPPCRPPARPRAGPPAAQDRPAPRRPPAARAAMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYGPAALATGTSAVPPKPGHFRGGHHRLCIRFRYYSATRSLFTFGVLFLFTFLLPFQGRHMVACMSGDGPGSWGLEGRGPSALGSQGALPAQPGLSWEPLAQRLSLANLGGCVHPARPSCGLHSSPCSLLPRFESRVCGHSLPSCTCPFHVAFNCNHNILTPSFNKEGTSGML

SEQ ID NO: 36 - DNA, 1482 bp, Homo sapiens
ggccagagagcgagcgcgcaacggcggcgacggcggcgaccccaccgcgcatcctgccaggcctccggcgcccagcgccccacgcgcccccgcgcccccgcgcccccgcgcccctttcttcgcgcccccgcccctcggcccgccaggcccccttgccggccacccgccaggccccgcgccggcccgcccgccgcccaggaccggcccgcgccccgcaggccgcccgccgcccgcgccgccatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactgaggccccgcagctctggccacagggacctctgcagtgccccctaagtgacccggacacttccgagggggccatcaccgcctgtgtatataacgtttccggtattactctgctacacgtagcctttttacttttggggttttgtttttgttctgaactttcctgttaccttttcagggctgacgtcacatgtaggtggcgtgtatgagtggagacgggcctgggtcttggggactggagggcaggggtccttctgccctggggtcccagggtgctctgcctgctcagccaggcctctcctgggagccactcgcccagagactcagcttggccaacttggggggctgtgtccacccagcccgcccgtcctgtgggctgcacagctcaccttgttccctcctgccccggttcgagagccgagtctgtgggcactctctgccttcatgcacctgtcctttctaacacgtcgccttcaactgtaatcacaacatcctgactccgtcatttaataaagaaggaacatcaggcatgcta

SEQ ID NO: 37 - protein, 1484 aa, Homo sapiens
KGRPWPLWSGPRAAARPFPRGPLLRRAPGPHPSQHPAPRALPAGSSRSHGAGAAVSTMELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPVTRRPSPQKPCVLREQGRPDFCWHQEVGGPSDHFQGNLPCQEPVLRNLPSCLSSQMAGRGPASLEEEQHWGVFVDSEALPNETLGSSGCHSPAWPFPSRSWVLKALGKLAEGKRPGSVEQKRPIQRLSLKPSTAPHEEGTAMVSVSRLCTECFSVFLLFLFCFFKDEIKTQGE

SEQ ID NO: 38 - DNA, 4473 bp, Homo sapiens
aaggggaggtaaccctggcccctttggtcggggccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggag

SEQ ID NO: 39 - protein, 261 aa, Homo sapiens
MKLFAFIFICVKSCSLLFMLNGNPRPEQLNKASEFTGLMDNMRYLYDDKHVSETNIKAQEKFLQHDLLFKINGSKIDGSKILKTEFNNKSLSDKYKNKNVDLFGTNYYNQCYFSADNMELNDGRLIEKTCMYGGVTEHDGNQIDKNNLTDNSHNILIKVYENERNTLSFDISTNKKNITAQEIDYKVRNYLLKHKNLYKFNSSPYETGYIKFIEGNGHSFWYDMMPESGEKFYPTKYLLIYNDNKTVESKSINVEVHLTKK

SEQ ID NO: 40 - DNA, 786 bp, Homo sapiens
atgaagttatttgcttttatcttcatatgtgttaagtcttgcagcttactatttatgttaaatggcaatcctagaccagaacaattgaataaagcgagtgaattcactggtctaatggataatatgaggtatttgtatgacgataaacacgtatcagaaacaaacattaaagcccaagagaagtttttacaacatgatttattatttaaaataaatggctctaaaattgatggttctaaaattttaaaaacagaatttaataataaaagcctttcggataaatacaaaaataaaaacgtagatttgtttgggacaaactattataatcaatgctatttttcagcggataatatggaattaaatgatggtagactaattgaaaaaacgtgtatgtatggcggtgtgaccgagcatgatggaaatcaaatagataaaaataatctaactgataactctcataatatcttaattaaagtatacgaaaacgaaagaaacacattatcttttgatatatctaccaataagaaaaacataacagcacaagaaatagattataaagttagaaactatttacttaagcataaaaatttatataaatttaatagttcgccttatgagactggctatataaagtttatcgaaggaaatggtcattctttttggtatgatatgatgcctgaatctggtgaaaaattttatccgactaaatatttactaatttataatgataataagacagttgagagtaaatctattaatgtagaagttcatttaaccaaaaaataa

SEQ ID NO: 41 - protein, 1517 aa, Homo sapiens
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTHHHHHHMAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 42 - DNA, 4551 bp, Homo sapiens
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacccaccaccaccaccaccacatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatg

SEQ ID NO: 43 - protein, 1952 aa, Artificial Sequence
  Synthetic Construct
PWPLWSGPRAAARPFPRGPLLRRAPGPHPSQHPAPRALPAGSSRSHGAGAAVSTMELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPVTRRPSPQKPCVLREQGRPDFCWHQEVGGPSDHFQGNLPCQEPVLRNLPSCLSSQMAGRGPASLEEEQHWGVFVDSEALPNETLGSSGCHSPAWPFPSRSWVLKALGKLAEGKRPGSVEQKRPIQRLSLKPSTAPHEEGTAMVSVSRLCTECFSVFLLFLFCFFKDEIKTQGEDYKDDDDKGQRASAQRRRRRRPHRASCQASGAQRPTRPRAPAPPRPFLRAPAPRPARPPCRPPARPRAGPPAAQDRPAPRRPPAARAAMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYGPAALATGTSAVPPKPGHFRGGHHRLCIRFRYYSATRSLFTFGVLFLFTFLLPFQGRHMVACMSGDGPGSWGLEGRGPSALGSQGALPAQPGLSWEPLAQRLSLANLGGCVHPARPSCGLHSSPCSLLPRFESRVCGHSLPSCTCPF

SEQ ID NO: 44 - DNA, 5979 bp, Artificial Sequence
  Synthetic Construct
aaggggaggtaaccctggcccctttggtcggggccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggaggactacaaggacgacgacgacaagggccagagagcgagcgcgcaacggcggcgacggcggcgaccccaccgcgcatcctgccaggcctccggcgcccagcgccccacgcgcccccgcgcccccgcgcccccgcgcccctttcttcgcgcccccgcccctcggcccgccaggcccccttgccggccacccgccaggccccgcgccggcccgcccgccgcccaggaccggcccgcgccccgcaggccgcccgccgcccgcgccgccatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactgaggccccgcagctctggccacagggacctctgcagtgccccctaagtgacccggacacttccgagggggccatcaccgcctgtgtatataacgtttccggtattactctgctacacgtagcctttttacttttggggttttgtttttgttctgaactttcctgttaccttttcagggctgacgtcacatgtaggtggcgtgtatgagtggagacgggcctgggtcttggggactggagggcaggggtccttctgccctggggtcccagggtgctctgcctgctcagccaggcctctcctgggagccactcgcccagagactcagcttggccaacttggggggctgtgtccacccagcccgcccgtcctgtgggctgcacagctcaccttgttccctcctgccccggttcgagagccgagtctgtgggcactctctgccttcatgcacctgtcctttctaacacgtcgccttcaactgtaatcacaacatcctgactccgtcatttaataaagaaggaacatcaggcatgcta

SEQ ID NO: 45 - protein, 1028 aa, Artificial Sequence
  Synthetic Construct
MKLFAFIFICVKSCSLLFMLNGNPRPEQLNKASEFTGLMDNMRYLYDDKHVSETNIKAQEKFLQHDLLFKINGSKIDGSKILKTEFNNKSLSDKYKNKNVDLFGTNYYNQCYFSADNMELNDGRLIEKTCMYGGVTEHDGNQIDKNNLTDNSHNILIKVYENERNTLSFDISTNKKNITAQEIDYKVRNYLLKHKNLYKFNSSPYETGYIKFIEGNGHSFWYDMMPESGEKFYPTKYLLIYNDNKTVESKSINVEVHLTKKRSRRASWSGSTATRATTAWPRWARSSRRTPASRPWSTPTSWRRSSPRWPPPATAPTSSSGPTTGSAATPRAACWPRSPPTRPSRTSCTPSPGTPGTTASSPTPSPWRPASTTRTCCPTPPRPGRRSPPWTRSRPRARAPCSTCRSPTSPGPSPPTAATPSSTRTASTTSRTWAWTTPAPRPAPSWWTSEQAHERRHRLQHRRGRLQQGRDRHDHQRPLGLEQHRHQQGELRRDRAAHLQGPAQQALRGRAERRHQRRQPQQGAGQGVPGELPADRRGPGGREQGQAPGRRGPEELRGGAGQGPQDRRHHGERPEGRDHAQHPPDERLLVRREDRRDQRRQRQADRGRGPEGRPDQQQQQQQQQQQQQQPGHRGQGPAYSRLHLYPAPGPASPSCARPPVPHPFGPGVSPHHAGQRRHQVHQIPAVRILHLLACRDCCPCHWTMAPIRLSDQEHLRARNFQLLHRSLYSDRSRRPHDAGGLPGLLRGCAGVPVHAGTVLRLPLGDIRHNSCGHLGIFPQGGDGSPGVLQGHLQQAENQGAPAGNAESHPLCVELLWFGWGRGTVYLRHLPQEGRTRNLHREVLSCHQRGLRQIPHHRRSGHRHCRGHDIWHDLQYDLVLCYPQEPRDGLESAYIPEQESLPMKIGGIFCLFVLFCLLFVVCFFATNFSIHSALLDKSSYFMFVFCFIQYHLLSGGFGLLWFIFFQLFVFACYIKQKSCNERYYICTLDKILYIKEFFCLIDTNVYQLSSCNLYRQFDTKIMTMSKKKK

SEQ ID NO: 46 - DNA, 3191 bp, Artificial Sequence
  Synthetic Construct
atgaagttatttgcttttatcttcatatgtgttaagtcttgcagcttactatttatgttaaatggcaatcctagaccagaacaattgaataaagcgagtgaattcactggtctaatggataatatgaggtatttgtatgacgataaacacgtatcagaaacaaacattaaagcccaagagaagtttttacaacatgatttattatttaaaataaatggctctaaaattgatggttctaaaattttaaaaacagaatttaataataaaagcctttcggataaatacaaaaataaaaacgtagatttgtttgggacaaactattataatcaatgctatttttcagcggataatatggaattaaatgatggtagactaattgaaaaaacgtgtatgtatggcggtgtgaccgagcatgatggaaatcaaatagataaaaataatctaactgataactctcataatatcttaattaaagtatacgaaaacgaaagaaacacattatcttttgatatatctaccaataagaaaaacataacagcacaagaaatagattataaagttagaaactatttacttaagcataaaaatttatataaatttaatagttcgccttatgagactggctatataaagtttatcgaaggaaatggtcattctttttggtatgatatgatgcctgaatctggtgaaaaattttatccgactaaatatttactaatttataatgataataagacagttgagagtaaatctattaatgtagaagttcatttaaccaaaaaataatgaagatcgaggagggcaagctggtgatctggatcaacggcgacaagggctacaacggcctggccgaggtgggcaagaagttcgagaaggacaccggcatcaaggtgaccgtggagcaccccgacaagctggaggagaagttcccccaggtggccgccaccggcgacggccccgacatcatcttctgggcccacgacaggttcggcggctacgcccagagcggcctgctggccgagatcacccccgacaaggccttccaggacaagctgtaccccttcacctgggacgccgtgaggtacaacggcaagctgatcgcctaccccatcgccgtggaggccctgagcctgatctacaacaaggacctgctgcccaacccccccaagacctgggaggagatccccgccctggacaaggagctgaaggccaagggcaagagcgccctgatgttcaacctgcaggagccctacttcacctggcccctgatcgccgccgacggcggctacgccttcaagtacgagaacggcaagtacgacatcaaggacgtgggcgtggacaacgccggcgccaaggccggcctgaccttcctggtggacctgatcagaacaagcacatgaacgccgacaccgactacagcatcgccgaggccgccttcaacaagggcgagaccgccatgaccatcaacggcccctgggcctggagcaacatcgacaccagcaaggtgaactacggcgtgaccgtgctgcccaccttcaagggccagcccagcaagcccttcgtgggcgtgctgagcgccggcatcaacgccgccagccccaacaaggagctggccaaggagttcctggagaactacctgctgaccgacgagggcctggaggccgtgaacaaggacaagcccctgggcgccgtggccctgaagagctacgaggaggagctggccaaggaccccaggatcgccgccaccatggagaacgcccagaagggcgagatcatgcccaacatcccccagatgagcgccttctggtacgccgtgaggaccgccgtgatcaacgccgccagcggcaggcagaccgtggacgaggccctgaaggacgcccagaccaacagcagcagcaacaacaacaacaacaacaacaacaacaacctgggcatcgagggcagggaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcaaaaaaaaaaaaaaa

SEQ ID NO: 47 - protein, 2484 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTHHHHHHMAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVMEQKLISEEDLELLCYSELQPQPRYGSQQPPARAARTPGPARGSVPRTWQCPDICVPLKSHPAPGRLRAGDMQHLLPAQVVGHRDPVAKGVAPAWEQPEGVTEQCARRPTNVLFKLPWAVNSNLPHRVLDSRTGGTGTPPLLAASGQEPYPTLPGGGWGTPGQPHRGAAPWGEGAETGASCGGARGHDHGAGEERSPWSQFLVPHTGPAAPRAGAVEHLGPLPAPDLCPASDSPTTCQPPGPRGGHAGDRGLFPGRAVPSLGGPGPPGTGGPEVEPHSHLWQRLLLGQGLSQCDRRGRGHPAADVCSNTGEPEPGDTADSDHLQLSGAQRDSDEARGLRRDRGDSEVGPPSQGDAEWGSSPATGPEGPAPAEGHPRGQRAQLLLLCNPGGGRPAYTQEPDPGASCPVWPPTGREGLSGKLDVARKFPADSNVPGLGEPIARAQVSKGWHFPTAHRGISDCHSRSGHLPLSGQEHSRGGHPQGDRECALPPVDCHHHCGSSRSHNGHCRPQHVPLPPAEDQEIQTTTGPKRDPHETEHTSHASLNLSRDRASSSAFPYWWQWCHTEQSGRHMPCSYTYRPWDAGGQGIVLSQIQQHLGPWYLHTNTRPRISVVTLSQEEGARLKTLMDVKVPDERGSGGGDIAPPGHTTGKYNLLPIGYAEAPQTYRRSGPPTCVASKHKGPHFLTDASLGTAVYPQPLMICIYSFVILPAIYVSFMAKTVSGLTELPVLITFKVTRYSCTGCTLQESAWQKDQMGLGLLIGQPAFPQKEFFYRHKSTIWTGNGYRFRDYPVRPYSSLPPKTDTFVSHLPTHIHFCQCSQHSAVMSGHECPGNMPKLCLVLLSCLHFTGSLHYAAPVSCSDQGPASSGEGGQGIGGLPPSFGSLIRVCVCVYVTSSRSVTQAGVQWCNHGSLQSPFGLKSSHLSLLSSWDHRLTTPHLANLIFFFFPETGSRNIAQTSFVLVNKAFSTA

SEQ ID NO: 48 - DNA, 7548 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacccaccaccaccaccaccacatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatggaacaaaaactcatctcagaagaggatctggagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 49 - protein, 388 aa, Artificial Sequence; identical to SEQ ID NO: 5
  Synthetic Construct

SEQ ID NO: 50 - DNA, 4473 bp, Artificial Sequence; identical to SEQ ID NO: 38
  Synthetic Construct

SEQ ID NO: 51 - protein, 1720 aa, Artificial Sequence
  Synthetic Construct
MKLFAFIFICVKSCSLLFMLNGNPRPEQLNKASEFTGLMDNMRYLYDDKHVSETNIKAQEKFLQHDLLFKINGSKIDGSKILKTEFNNKSLSDKYKNKNVDLFGTNYYNQCYFSADNMELNDGRLIEKTCMYGGVTEHDGNQIDKNNLTDNSHNILIKVYENERNTLSFDISTNKKNITAQEIDYKVRNYLLKHKNLYKFNSSPYETGYIKFIEGNGHSFWYDMMPESGEKFYPTKYLLIYNDNKTVESKSINVEVHLTKKRSRRASWSGSTATRATTAWPRWARSSRRTPASRPWSTPTSWRRSSPRWPPPATAPTSSSGPTTGSAATPRAACWPRSPPTRPSRTSCTPSPGTPGTTASSPTPSPWRPASTTRTCCPTPPRPGRRSPPWTRSRPRARAPCSTCRSPTSPGPSPPTAATPSSTRTASTTSRTWAWTTPAPRPAPSWWTSEQAHERRHRLQHRRGRLQQGRDRHDHQRPLGLEQHRHQQGELRRDRAAHLQGPAQQALRGRAERRHQRRQPQQGAGQGVPGELPADRRGPGGREQGQAPGRRGPEELRGGAGQGPQDRRHHGERPEGRDHAQHPPDERLLVRREDRRDQRRQRQADRGRGPEGRPDQQQQQQQQQQQQQQPGHRGQDQPTAACICIQRQVPPVPAARAPQSRTRSAQAKLALTMPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVSQLTSLSRKVYPRLVGFFVCLFCFVCCLLFVFLPLILVFILHCIKAEVTLCLSFNASFNIDICSAGGLVCFGLYFFSCLFLLVILSRNPAMKGTIFARLTRYCTKNFFVFKIQMSINFNQVVTYIEDNLIHNKKLQCQKKKKHHHHHHTRKQACTQTRHHASPRPLAPAPGSRILGFLLKMGFAPNPLRLGGGLRLDPLHLQSPRWPGKLHPVPQSVQEHLEVWDKTLKHKGWEGSFAEKGAIPGRQEELHTEYPPGAPQQWSAGAEDGVQDEMDGTNTPQCLKAFSTSYPAPSRNSRVPGSHSDLLAEFLLLWVSDPIAVAPRGGSNEAGCCHLDLLDHQVCLHPERAQVLPTVESPWEDCDLPASGCRWEVPLQHGAAEREASSQEGDHSDSKPHADSRRRHSDPFLLQFQPQCYPVMETPWRLGGAIAWGAEDPKRWLGQHNHRLRSLLVLVGLPCRPECPVCPPRREGPENQAPFRDSLWKLGQPPMLLKQPPQRSPVLLGEKWQASGERKPAEFLHLPRRCWELQLLGEQLHRTDSVQGLDTSAVCTQEAACVHEPGGPSDGGEECNPDLERRQPSRLPLHLVLEPKPPLPQPEAEIGAGEGPALGCLLVPGDQQCGQGPFASQHPHRLLPGDHRQASGCGTRVLPRHPHPGNLWAQAPATLEEDTEPAGASGEFQRPELLCEEKGKGPPLRPPLPGMLQSNDGRWHLHHPALSRDEHTTNWRCRVLRDAETSPGLRHGHLFSIAQAPSGHYENVIPDFPEDEGIHYSELIQFGVGERPQAQENVDYVILKHHWMGCSRGTGGSGGQGSPRVFPR

SEQ ID NO: 52 - DNA, 5323 bp, Artificial Sequence
  Synthetic Construct
atgaagttatttgcttttatcttcatatgtgttaagtcttgcagcttactatttatgttaaatggcaatcctagaccagaacaattgaataaagcgagtgaattcactggtctaatggataatatgaggtatttgtatgacgataaacacgtatcagaaacaaacattaaagcccaagagaagtttttacaacatgatttattatttaaaataaatggctctaaaattgatggttctaaaattttaaaaacagaatttaataataaaagcctttcggataaatacaaaaataaaaacgtagatttgtttgggacaaactattataatcaatgctatttttcagcggataatatggaattaaatgatggtagactaattgaaaaaacgtgtatgtatggcggtgtgaccgagcatgatggaaatcaaatagataaaaataatctaactgataactctcataatatcttaattaaagtatacgaaaacgaaagaaacacattatcttttgatatatctaccaataagaaaaacataacagcacaagaaatagattataaagttagaaactatttacttaagcataaaaatttatataaatttaatagttcgccttatgagactggctatataaagtttatcgaaggaaatggtcattctttttggtatgatatgatgcctgaatctggtgaaaaattttatccgactaaatatttactaatttataatgataataagacagttgagagtaaatctattaatgtagaagttcatttaaccaaaaaataatgaagatcgaggagggcaagctggtgatctggatcaacggcgacaagggctacaacggcctggccgaggtgggcaagaagttcgagaaggacaccggcatcaaggtgaccgtggagcaccccgacaagctggaggagaagttcccccaggtggccgccaccggcgacggccccgacatcatcttctgggcccacgacaggttcggcggctacgcccagagcggcctgctggccgagatcacccccgacaaggccttccaggacaagctgtaccccttcacctgggacgccgtgaggtacaacggcaagctgatcgcctaccccatcgccgtggaggccctgagcctgatctacaacaaggacctgctgcccaacccccccaagacctgggaggagatccccgccctggacaaggagctgaaggccaagggcaagagcgccctgatgttcaacctgcaggagccctacttcacctggcccctgatcgccgccgacggcggctacgccttcaagtacgagaacggcaagtacgacatcaaggacgtgggcgtggacaacgccggcgccaaggccggcctgaccttcctggtggacctgatcagaacaagcacatgaacgccgacaccgactacagcatcgccgaggccgccttcaacaagggcgagaccgccatgaccatcaacggcccctgggcctggagcaacatcgacaccagcaaggtgaactacggcgtgaccgtgctgcccaccttcaagggccagcccagcaagcccttcgtgggcgtgctgagcgccggcatcaacgccgccagccccaacaaggagctggccaaggagttcctggagaactacctgctgaccgacgagggcctggaggccgtgaacaaggacaagcccctgggcgccgtggccctgaagagctacgaggaggagctggccaaggaccccaggatcgccgccaccatggagaacgcccagaagggcgagatcatgcccaacatcccccagatgagcgccttctggtacgccgtgaggaccgccgtgatcaacgccgccagcggcaggcagaccgtggacgaggccctgaaggacgcccagaccaacagcagcagcaacaacaacaacaacaacaacaacaacaacctgggcatcgagggcaggaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcaaaaaaaaaaaaaacaccaccaccaccaccacacgcggaaacaggcttgcacccagacacgacaccatgcatctcctcggcccctggctcctgctcctggttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggcttcaggagaattccagcggccagagcttctttgtgaggaataaaaaggttagaagggcccccctctctgaaggcccccactccctgggatgctacaatccaatgatggaagatggcattagctacaccaccctgcgctttcccgagatgaacataccacgaactggagatgcagagtcctcagagatgcagagacctcccccggactgcgatgacacggtcacttattcagcattgcacaagcgccaagtgggcactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagttttccccagac

SEQ ID NO: 53 - protein, 1201 aa, Artificial Sequence
  Synthetic Construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVMHHHHHHELLCYSELQPQPRYGSQQPPARAARTPGPARGSVPRTWQCPDICVPLKSHPAPGRLRAGDMQHLLPAQVVGHRDPVAKGVAPAWEQPEGVTEQCARRPTNVLFKLPWAVNSNLPHRVLDSRTGGTGTPPLLAASGQEPYPTLPGGGWGTPGQPHRGAAPWGEGAETGASCGGARGHDHGAGEERSPWSQFLVPHTGPAAPRAGAVEHLGPLPAPDLCPASDSPTTCQPPGPRGGHAGDRGLFPGRAVPSLGGPGPPGTGGPEVEPHSHLWQRLLLGQGLSQCDRRGRGHPAADVCSNTGEPEPGDTADSDHLQLSGAQRDSDEARGLRRDRGDSEVGPPSQGDAEWGSSPATGPEGPAPAEGHPRGQRAQLLLLCNPGGGRPAYTQEPDPGASCPVWPPTGREGLSGKLDVARKFPADSNVPGLGEPIARAQVSKGWHFPTAHRGISDCHSRSGHLPLSGQEHSRGGHPQGDRECALPPVDCHHHCGSSRSHNGHCRPQHVPLPPAEDQEIQTTTGPKRDPHETEHTSHASLNLSRDRASSSAFPYWWQWCHTEQSGRHMPCSYTYRPWDAGGQGIVLSQIQQHLGPWYLHTNTRPRISVVTLSQEEGARLKTLMDVKVPDERGSGGGDIAPPGHTTGKYNLLPIGYAEAPQTYRRSGPPTCVASKHKGPHFLTDASLGTAVYPQPLMICIYSFVILPAIYVSFMAKTVSGLTELPVLITFKVTRYSCTGCTLQESAWQKDQMGLGLLIGQPAFPQKEFFYRHKSTIWTGNGYRFRDYPVRPYSSLPPKTDTFVSHLPTHIHFCQCSQHSAVMSGHECPGNMPKLCLVLLSCLHFTGSLHYAAPVSCSDQGPASSGEGGQGIGGLPPSFGSLIRVCVCVYVTSSRSVTQAGVQWCNHGSLQSPFGLKSSHLSLLSSWDHRLTTPHLANLIFFFFPETGSRNIAQTSFVLVNKAFSTA

SEQ ID NO: 54 - DNA, 2967 bp, Artificial Sequence; identical to SEQ ID NO: 4
  Synthetic Construct

SEQ ID NO: 55 - protein, 1502 aa, Artificial Sequence
  Synthetic Construct
DQPTAACICIQRQVPPVPAARAPQSRTRSAQAKLALTMPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVSQLTSLSRKVYPRLVGFFVCLFCFVCCLLFVFLPLILVFILHCIKAEVTLCLSFNASFNIDICSAGGLVCFGLYFFSCLFLLVILSRNPAMKGTIFARLTRYCTKNFFVFKIQMSINFNQVVTYIEDNLIHNKKLQCQKKKKRLQGRRRQGFSPMKIKHILLLGVRHCMCPCLMLEMMHMKRLYMSNYPWVFISLRFSCCFIFPIRKRSKTVKSQITLAWYNLTAVLAIYMIISQGILAFSWMAHSAERKRTSVSQCMLPVKMKRKWTISTAELQYLNMRLSLFMGLTQLHLCMDQMMKMSLKRAWWRKTLSMLSTLAIVWLPMLVWKWYQILLAPKLISCSTFWMSRLLLENATLKIIKECVHSSKRVQCRPKASSSCPRLIRGYCTAKLIHIVISCVILGKWKVEKKPVFISNWKAGHPFKWMRLQHSSLKEQQVFQSQIQELNTRMRMLRMFYWKDYIIKDPNVISPLFQVACYLDLLYFCSHMLCGRLASLKDNTNLSYKKKTEETVGVISTVKAMMIKDFFQIERMENRLRLRNLKDTVYKKKILFGLLLLMILHIMSSCKGKISAMITLDRRTAKVIIQPKIISQLLNGRNTKAFNLFKKSKPLKISNESITELNYTGEVLDLKYYLPYVLASVKTPLGGQRFISNTSLILVQNMFFKNIIFRAVPKFSNEWTIITLKPFIYNTFPTGCVPTTIFFQQTMNIIVLAKLANFRLNMYTGLSLVKLLDNYFFIIMDFTIFLSVYIHVFLCRYIFTILPIYSSYNTPLSSIPRSNLQIFCYILKQKIVSVALYLIHADLENTETIPHLNQSCAKTRGPILHMYYVLCKILTITQLFPWMFFVTLYKYKCYLTWKRRNKIHKFKFINLAERYFNLYTVNINETTAKFSNVKYFYSLFILYEVLFMTFWILKNFFKYNHFCNIYFMLMIIIAEYHFILCLHKRAVAEFTSVIGSDQIRRQCGKTILGKISLYEVPATSQPSLMKVICSQACSDGEECSEICEGIVVKCKHKTSTQSVYTQETLHLCIFKKLCIVIKRLVILSGEHLGSMKNQAPINCLLQDCLGNLHLVLSYSELLRNNYIFQVIAQKQSTMVVQHRPLRYKGNTITYWARFCPVQNSICYQLTLLLVSILKPYHFKKTGMGYSFLAGRLYNYVILKFNCSGLGSSESRQTYEICIIFVTEYRKFNIIDELKSKMKESKLFQNYLGILMYYDGCKVFCVSNKHIVKKRIIDIKKKK

SEQ ID NO: 56 - DNA, 3459 bp, Artificial Sequence; identical to SEQ ID NO: 16
  Synthetic Construct

SEQ ID NO: 57 - protein, 1026 aa, Artificial Sequence
  Synthetic Construct
GQRASAQRRRRRRPHRASCQASGAQRPTRPRAPAPPRPFLRAPAPRPARPPCRPPARPRAGPPAAQDRPAPRRPPAARAAMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYGPAALATGTSAVPPKPGHFRGGHHRLCIRFRYYSATRSLFTFGVLFLFTFLLPFQGRHMVACMSGDGPGSWGLEGRGPSALGSQGALPAQPGLSWEPLAQRLSLANLGGCVHPARPSCGLHSSPCSLLPRFESRVCGHSLPSCTCPFHVAFNCNHNILTPSFNKEGTSGMLEQKLISEEDLTRAPSLHSANCSLAHLPPLHHDHLQPPVHLLQLHEGLLRHRGRHRGRLQPHLLRPGRRVLPRPQHLRGRPVCLILPLLLWGSLRAGGRLWRWLQQQQQQLWWLWGRIWWWPWCWLGWWLWWWLCWWWASGGQEGDHAEPQPPGLLPGQGACSGGGQRRPGSEDPLVPEAAACDQRLQSLLQDHGPEEQDSHSHSGQCQCPSADQCPSGRGLPHQVDRVEPAHECGSRHQWPAQGAGRTDPGQSPGDADEPEGGAGLPEEEPRGGDECPERPGGWRCQCGDGRCTWRGPEPHSERDAPVEDGREEPQGCRGMVLHQDRGAEPRGGHQQRAGAERQERDLGAPAHHAEPGDAAVPAQHESIPGEQPGGDQRSLLHAAGPDPGDDWQRGGAAGPAPLRDGAAEPGVQDPAGREDAAGAGDRHLPPPAGGRGRPPLLLPVLLWIAVIQRCDLLQPPNPHQGHGCARWQGGVHPRAGPSHQELRLPSPAQAEAPRVDTDPTGRSPLLPKHFTAGPCFTLTPSWQSIQLHYLSCI

SEQ ID NO: 58 - DNA, 3148 bp, Artificial Sequence
  Synthetic Construct
ggccagagagcgagcgcgcaacggcggcgacggcggcgaccccaccgcgcatcctgccaggcctccggcgcccagcgccccacgcgcccccgcgcccccgcgcccccgcgcccctttcttcgcgcccccgcccctcggcccgccaggcccccttgccggccacccgccaggccccgcgccggcccgcccgccgcccaggaccggcccgcgccccgcaggccgcccgccgcccgcgccgccatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactgaggccccgcagctctggccacagggacctctgcagtgccccctaagtgacccggacacttccgagggggccatcaccgcctgtgtatataacgtttccggtattactctgctacacgtagcctttttacttttggggttttgtttttgttctgaactttcctgttaccttttcagggctgacgtcacatgtaggtggcgtgtatgagtggagacgggcctgggtcttggggactggagggcaggggtccttctgccctggggtcccagggtgctctgcctgctcagccaggcctctcctgggagccactcgcccagagactcagcttggccaacttggggggctgtgtccacccagcccgcccgtcctgtgggctgcacagctcaccttgttccctcctgccccggttcgagagccgagtctgtgggcactctctgccttcatgcacctgtcctttctaacacgtcgccttcaactgtaatcacaacatcctgactccgtcatttaataaagaaggaacatcaggcatgctagaacaaaaactcatctcagaagaggatctgacccgagcaccttctcttcactcagccaactgctcgctcgctcacctccctcctctgcaccatgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctgcgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcattatctgagttgcataa